(19) United States
(12) Patent Application Publication
(10) Pub. No.: US 2020/0238097 A1
(43) Pub. Date: Jul. 30, 2020

(54) Head-Worn Mobile Neurostimulation Device

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 16/838,541

(22) Filed: Apr. 2, 2020

Related U.S. Application Data
(63) Continuation-in-part of application No. 16/554,029, filed on Aug. 28, 2019.
Application No. 16/554,029 is a continuation-in-part of application No. 15/236,401, filed on Aug. 13, 2016.
Application No. 15/236,401 is a continuation-in-part of application No. 16/022,987, filed on Jun. 29, 2018.
Application No. 16/022,987 is a continuation-in-part of application No. 15/136,948, filed on Apr. 24, 2016, now Pat. No. 10,234,942.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
(60) Provisional application No. 62/972,692, filed on Feb. 11, 2020.
(60) Provisional application No. 62/851,917, filed on May 23, 2019.
(60) Provisional application No. 62/851,904, filed on May 23, 2019.
(60) Provisional application No. 62/837,712, filed on Apr. 23, 2019.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/322,594, filed on Apr. 14, 2016.
(60) Provisional application No. 62/303,126, filed on Mar. 3, 2016.
(60) Provisional application No. 62/169,661, filed on Jun. 2, 2015.
(60) Provisional application No. 62/160,172, filed on May 12, 2015.
(60) Provisional application No. 62/089,696, filed on Dec. 9, 2014.
(60) Provisional application No. 62/017,615, filed on Jun. 26, 2014.
(60) Provisional application No. 61/939,244, filed on Feb. 12, 2014.
(60) Provisional application No. 61/932,517, filed on Jan. 28, 2014.
(60) Provisional application No. 61/932,517, filed on Jan. 28, 2014.

(51) Int. Cl. A61N 2/00; G16H 40/60

(52) U.S. Cl. CPC A61N 2/006; G16H 40/60

(57) ABSTRACT

This invention is a wearable neurostimulation device which transmits selected patterns of low-intensity electromagnetic energy into a person's head for therapeutic purposes. This device can include a wearable ring and/or band which at least partially encircles a person's head and has flexible electroconductive prongs and/or teeth which protrude into and/or under the person's hair.

### CROSS-REFERENCE TO RELATED APPLICATIONS

This application claims the priority benefit of U.S. provisional application 62/972,692 filed on 2020 Feb. 11. This application is a continuation in part of U.S. application Ser. No. 16/554,029 filed on 2019 Aug. 28. This application claims the priority benefit of U.S. provisional application 62/851,917 filed on 2019 May 23. This application claims the priority benefit of U.S. provisional application 62/851,904 filed on 2019 May 23. This application claims the priority benefit of U.S. provisional application 62/837,712 filed on 2019 Apr. 23. This application is a continuation in part of U.S. application Ser. No. 15/236,401 filed on 2016 Aug. 13.

U.S. patent application Ser. No. 16/554,029 claimed the priority benefit of U.S. provisional application 62/851,904 filed on 2019 May 23. U.S. patent application Ser. No. 16/554,029 claimed the priority benefit of U.S. provisional application 62/796,901 filed on 2019 Jan. 25. U.S. patent application Ser. No. 16/554,029 claimed the priority benefit of U.S. provisional application 62/791,838 filed on 2019 Jan. 13. U.S. patent application Ser. No. 16/554,029 was a continuation in part of U.S. application Ser. No. 16/022,987 filed on 2018 Jun. 29. U.S. patent application Ser. No. 16/022,987 was a continuation in part of U.S. application Ser. No. 15/136,948 filed on 2016 Apr. 24 which issued as U.S. patent Ser. No. 10/234,942 on 2019 Mar. 19. U.S. patent application Ser. No. 15/236,401 was a continuation in part of U.S. application Ser. No. 15/136,948 filed on 2016 Apr. 24 which issued as U.S. patent Ser. No. 10/234,942 on 2019 Mar. 19. U.S. patent application Ser. No. 15/236,401 was a continuation in part of U.S. application Ser. No. 14/599,522 filed on 2015 Jan. 18 which issued as U.S. Pat. No. 9,814,426 on 2017 Nov. 14.

U.S. patent application Ser. No. 15/136,948 claimed the priority benefit of U.S. provisional application 62/322,594 filed on 2016 Apr. 14. U.S. patent application Ser. No. 15/136,948 claimed the priority benefit of U.S. provisional application 62/303,126 filed on 2016 Mar. 3. U.S. patent application Ser. No. 15/136,948 claimed the priority benefit of U.S. provisional application 62/169,661 filed on 2015 Jun. 2. U.S. patent application Ser. No. 15/136,948 claimed the priority benefit of U.S. provisional application 62/160,172 filed on 2015 May 12. U.S. patent application Ser. No. 15/136,948 was a continuation in part of U.S. application Ser. No. 14/599,522 filed on 2015 Jan. 18 which issued as U.S. Pat. No. 9,814,426 on 2017 Nov. 14.

U.S. patent application Ser. No. 14/599,522 claimed the priority benefit of U.S. provisional application 62/089,696 filed on 2014 Dec. 9. U.S. patent application Ser. No. 14/599,522 was a continuation in part of U.S. application Ser. No. 14/562,719 filed on 2014 Dec. 7 which issued as U.S. patent Ser. No. 10/130,277 on 2018 Nov. 20. U.S. patent application Ser. No. 14/599,522 claimed the priority benefit of U.S. provisional application 62/017,615 filed on 2014 Jun. 26. U.S. patent application Ser. No. 14/599,522 claimed the priority benefit of U.S. provisional application 61/939,244 filed on 2014 Feb. 12. U.S. patent application Ser. No. 14/599,522 claimed the priority benefit of U.S. provisional application 61/932,517 filed on 2014 Jan. 28. U.S. patent application Ser. No. 14/562,719 claimed the priority benefit of U.S. provisional application 61/932,517 filed on 2014 Jan. 28.

The entire contents of these applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable neurostimulation devices.

### INTRODUCTION

There are numerous potential applications for a non-invasive neurostimulation device with electrodes which transmit electromagnetic energy into a person's head. The applications include: alleviation of pain; addiction treatment; alleviating headaches; reducing body tremors; reducing seizures; addressing stress and anxiety; treating depression; increasing focus or energy level; reducing hunger; improving sleep; addressing attention-related disorders; and neurological rehabilitation.

### REVIEW OF THE RELEVANT ART

There has been considerable innovation in wearable devices for neurostimulation and neuromodulation during the past several years. For example, U.S. patent applications 20160346545 (Pal et al., Dec. 1, 2016, “Apparatuses and Methods for Neuromodulation”) and 20170224990 (Goldwasser et al., Aug. 10, 2017, “Apparatuses and Methods for Neuromodulation”), and also patent 10485972 (Pal et al., Nov. 26, 2019, “Apparatuses and Methods for Neuromodulation”), disclose apparatuses and methods for applying transdermal electrical stimulation to a person to enhance a concurrent sensory experience. U.S. patent applications 20170197081 (Charlesworth et al., Jul. 13, 2017, “Apparatuses and Methods for Transdermal Electrical Stimulation of Nerves to Modify or Induce a Cognitive State”) and 20190336765 (Charlesworth et al., Nov. 7, 2019, “Apparatuses and Methods for Transdermal Electrical Stimulation of Nerves to Modify or Induce a Cognitive State”), and also patent 10293161 (Charlesworth et al., May 21, 2019, “Apparatuses and Methods for Transdermal Electrical Stimulation of Nerves to Modify or Induce a Cognitive State”), disclose a portable transdermal electrical stimulation applicator for modifying a person's cognitive state by applying stimulation to skin.

U.S. patent application 20150335877 (Jeffery et al., Nov. 26, 2015, “Cantilever Electrodes for Transdermal and Transcranial Stimulation”) discloses cantilever electrode apparatuses for wearable neuromodulation devices. U.S. patent application 20150174418 (Tyler et al., Jun. 25, 2015, “Device and Methods for Noninvasive Neuromodulation Using Targeted Transcranial Electrical Stimulation”) discloses a system for transcranial electrical stimulation with a computer model that can be adjusted to a person's head in response to one or more input parameters. U.S. patent application 20150328461 (Charlesworth et al., Nov. 19, 2015, “Methods and Apparatuses for Amplitude-Modulated Ensemble Waveforms for Neurostimulation”) and U.S. Pat. No. 9,517,351 (Charlesworth et al., Dec. 13, 2016, “Methods and Apparatuses for Amplitude-Modulated Ensemble Waveforms for Neurostimulation”) disclose methods and apparatuses for amplitude modulation of an ensemble waveform to modify a user's cognitive state by transdermal electrical stimulation (TES).

U.S. patent application 20150328467 (Demers et al., Nov. 19, 2015, “Methods and Apparatuses for Control of a Wearable Transdermal Neurostimulator to Apply Ensemble Waveforms”) and U.S. Pat. No. 9,393,430 (Demers et al., Jul. 19, 2016, “Methods and Apparatuses for Control of a Wearable Transdermal Neurostimulator to Apply Ensemble Waveforms”) disclose methods and apparatuses for controlling a wearable neurostimulator from a control apparatus. U.S. patent application 20160346530 (Jeffery et al., Dec. 1, 2016, “Methods and Apparatuses for Transdermal Electrical Stimulation”) and patent Ser. No. 10/426,945 (Tyler et al., Oct. 1, 2019, “Methods and Apparatuses for Transdermal Stimulation of the Outer Ear”) disclose Transdermal Electrical Stimulation (TES) applicators that are attached to a person's ear. U.S. patent application 20150335876 (Jeffery et al., Nov. 26, 2015, “Methods for Attaching and Wearing a Neurostimulator”) and U.S. Pat. No. 9,333,334 (Jeffery et al., May 10, 2016, “Methods for Attaching and Wearing a Neurostimulator”) disclose methods for attaching a wearable neurostimulator to a person's head (or head and neck) using a cantilever electrode apparatus.

U.S. patent application 20160317809 (Pal et al., Nov. 3, 2016, “Methods for User Control of Neurostimulation to Modify a Cognitive State”) discloses methods which allows a person to control a neuromodulator to modify a cognitive state. U.S. patent application 20190321636 (Law et al., Oct. 24, 2019, “Streamlined and Pre-Set Neuromodulators”) discloses limited-number-of-use neuromodulator apparatuses that may be comfortably worn on the skin of a person to non-invasively apply transdermal electrical stimulation (TES). U.S. patent applications 20170182285 (Tyler et al., Jun. 29, 2017, “Systems and Methods for Transdermal Electrical Stimulation to Improve Sleep”) and 20170368297 (Tyler et al., Dec. 28, 2017, “Systems and Methods for Transdermal Electrical Stimulation to Improve Sleep”), and also patent 10537703 (Tyler et al., Jan. 21, 2020, “Systems and Methods for Transdermal Electrical Stimulation to Improve Sleep”) disclose methods and apparatuses for improving sleep by transdermal electrical stimulation (TES).

U.S. Pat. No. 9,956,405 (Goldwasser et al., May 1, 2018, “Transdermal Electrical Stimulation at the Neck to Induce Neuromodulation”) discloses methods and apparatuses for the application of transdermal electrical stimulation (TES) in order to modulate a person's cognitive state to induce a state of calm or relaxation. U.S. patent applications 20150005840 (Pal et al., Jan. 1, 2015, “Transdermal Electrical Stimulation Methods for Modifying or Inducing Cognitive State”), 20150005841 (Pal et al., Jan. 1, 2015, “Transdermal Electrical Stimulation Devices for Modifying or Inducing Cognitive State”), and 20150174403 (Pal et al., Jun. 25, 2015, “Transdermal Electrical Stimulation Devices for Modifying or Inducing Cognitive State”), and U.S. Pat. No. 9,002,458 (Pal et al., Apr. 7, 2015, “Transdermal Electrical Stimulation Devices for Modifying or Inducing Cognitive State”), U.S. Pat. No. 9,014,811 (Pal et al., Apr. 21, 2015, “Transdermal Electrical Stimulation Methods for Modifying or Inducing Cognitive State”), and U.S. Pat. No. 9,233,244 (Pal et al., Jan. 12, 2016, “Transdermal Electrical Stimulation Devices for Modifying or Inducing Cognitive State”) disclose portable transdermal electrical stimulation (TES) applicators for modifying a person's cognitive state.

U.S. patent application 20150335888 (Demers et al., Nov. 26, 2015, “Transdermal Neurostimulator Adapted to Reduce Capacitive Build-Up”) and U.S. Pat. No. 9,474,891 (Demers et al., Oct. 25, 2016, “Transdermal Neurostimulator Adapted to Reduce Capacitive Build-Up”) disclose neurostimulators for application of transdermal electrical stimulation (TES) and methods of using them for comfortably inducing a cognitive effect. U.S. patent applications 20140148872 (Goldwasser et al., May 29, 2014, “Wearable Transdermal Electrical Stimulation Devices and Methods of Using Them”) and 20150088224 (Goldwasser et al., Mar. 26, 2015, “Wearable Transdermal Electrical Stimulation Devices and Methods of Using Them”), and U.S. Pat. No. 8,903,494 (Goldwasser et al., Dec. 2, 2014, “Wearable Transdermal Electrical Stimulation Devices and Methods of Using Them”) disclose devices, systems, and methods with self-contained, lightweight, and wearable components for transdermal electrical stimulation.

U.S. patent application 20150335875 (Goldwasser et al., Nov. 26, 2015, “Wearable Transdermal Neurostimulator Having Cantilevered Attachment”) and U.S. Pat. No. 9,393,401 (Goldwasser et al., Jul. 19, 2016, “Wearable Transdermal Neurostimulator Having Cantilevered Attachment”) disclose neurostimulator apparatuses for applying transdermal electrical stimulation that may be comfortably and securely worn on a variety of head shapes and sizes.

U.S. patent applications 20170360321 (Wolber et al., Dec. 21, 2017, “Electrical Interface System”) and 20200046247 (Wolber et al., Feb. 13, 2020, “Electrical Interface System”), and U.S. patent Ser. No. 10/485,443 (Wolber et al., Nov. 26, 2019, “Electrical Interface System”) disclose an electrical interface between a transducer and a transducer support device. U.S. patent applications 20170113033 (Wingeier et al., Apr. 27, 2017, “Electrode Positioning System and Method”) and 20190255313 (Wingeier et al., Aug. 22, 2019, “Electrode Positioning System and Method”) disclose a system for stimulating a user comprising a set of pads, a band, a bridge, and a set of links.

U.S. patent applications 20150065838 (Wingeier et al., Mar. 5, 2015, “Electrode System for Electrical Stimulation”), 20160022981 (Wingeier et al., Jan. 28, 2016, “Electrode System for Electrical Stimulation”), 20160361532 (Wingeier et al., Dec. 15, 2016, “Electrode System for Electrical Stimulation”), 20170021158 (Wingeier et al., Jan. 26, 2017, “Electrode System for Electrical Stimulation”), 20170065816 (Wingeier et al., Mar. 9, 2017, “Electrode System for Electrical Stimulation”), 20190175910 (Wingeier et al., Jun. 13, 2019, “Electrode System for Electrical Stimulation”), and 20190374766 (Wingeier et al., Dec. 12, 2019, “Electrode System for Electrical Stimulation”) disclose systems including a housing with an array of protrusions for electrically stimulating and/or detecting bioelectrical signals. U.S. patent applications 20190151654 (Wingeier et al., May 23, 2019, “System and Method for Individualizing Modulation”) and 20200061375 (Wingeier et al., Feb. 27, 2020, “System and Method for Individualizing Neuromodulation”) disclose a system for individualizing neuromodulation including a neuro stimulation device with a set of electrodes and an application on a user device.

U.S. patent applications 20180256887 (Wingeier et al., Sep. 13, 2018, “System for Electrical Stimulation”), 20180256888 (Wingeier et al., Sep. 13, 2018, “System for Electrical Stimulation”), and 20200094045 (Wingeier et al., Mar. 26, 2020, “System for Electrical Stimulation”), as well as U.S. patent Ser. No. 10/512,770 (Wingeier et al., Dec. 24, 2019, “System for Electrical Stimulation”) and Ser. No. 10/525,255 (Wingeier et al., Jan. 7, 2020, “System for Electrical Stimulation”) disclose an electrical stimulation system with one or more electrode assemblies including one or more electrodes and an electronics subsystem.

U.S. patent application 20180028802 (Lee et al., Feb. 1, 2018, “Dry Electrode”) discloses a dry electrode that electrically stimulates while contacting skin of a user. U.S. patent applications 20170224978 (Lee, Aug. 10, 2017, “Electrical Stimulation Device”) and 20190336766 (Lee, Nov. 7, 2019, “Electrical Stimulation Device”), patent Ser. No. 10/342,969 (Lee, Jul. 9, 2019, “Electrical Stimulation Device”) disclose an electrical stimulation device comprising a frame to be worn on the head; an electrode part formed of a conductive member; a cover unit; and a detachable patch.

U.S. patent applications 20140350431 (Hagedorn, Nov. 27, 2014, “Headgear with Displacable Sensors for Electrophysiology Measurement and Training”), 20170027467 (Hagedorn, Feb. 2, 2017, “Headgear with Displacable Sensors for Electrophysiology Measurement and Training”), and 20180020941 (Hagedorn, Jan. 25, 2018, “Headgear with Displaceable Sensors for Electrophysiology Measurement and Training”), and also U.S. Pat. No. 8,938,301 (Hagedorn, Jan. 20, 2015, “Headgear with Displaceable Sensors for Electrophysiology Measurement and Training”) and U.S. Pat. No. 9,788,747 (Hagedorn, Oct. 17, 2017, “Headgear with Displaceable Sensors for Electrophysiology Measurement and Training”) disclose headgear for electrophysiological data collection and neurostimulation/neuromodulation.

U.S. patent applications 20150190635 (Neuvonen et al., Jul. 9, 2015, “System and a Method for Transcranial Stimulation of a Head Region of a Subject”) and 20170151430 (Neuvonen et al., Jun. 1, 2017, “System and Method for Transcranial Stimulation of a Subject”), and U.S. Pat. No. 9,370,658 (Neuvonen et al., Jun. 21, 2016, “System and a Method for Transcranial Stimulation of a Head Region of a Subject”) disclose a system for transcranial stimulation of a head region of a subject including a plurality of electrodes coupled to an electrode drive arrangement.

U.S. patent applications 20170296295 (Wagner et al., Oct. 19, 2017, “Adjustable Headpiece with Anatomical Markers and Methods of Use Thereof”) and 20200054414 (Wagner et al., Feb. 20, 2020, “Adjustable Headpiece with Anatomical Markers and Methods of Use Thereof”) disclose an adjustable headpiece with anatomical markers.

U.S. patent application 20180239430 (Tadi et al., Aug. 23, 2018, “Brain Activity Measurement and Feedback System”) discloses a head set with a brain electrical activity (EEG) sensing device. U.S. patent applications 20170296121 (Dar et al., Oct. 19, 2017, “Headset for Neurostimulation and Sensing of Body Parameters”) and 20190022372 (Dar et al., Jan. 24, 2019, “Resilient Head Mounted Device for Neurostimulation and Sensing of Body Parameters”) disclose a headset to deliver electrical stimulation to the skin of a person's head. U.S. patent application 20190192069 (Garcia et al., Jun. 27, 2019, “Sleep Stage Prediction and Intervention Preparation Based Thereon”) discloses a system for prediction of a sleep stage and an intervention preparation in advance of the sleep stage's occurrence.

U.S. patent application 20190282812 (Simons et al., Sep. 19, 2019, “Method for Non-Invasive Enhancement of Deep Sleep”) discloses an apparatus, system, and method for targeted sleep enhancement. U.S. patent application 20200094054 (Sharma et al., Mar. 26, 2020, “Auricular Nerve Stimulation to Address Patient Disorders, and Associated Systems and Methods”) discloses auricular nerve stimulation techniques for addressing patient disorders. U.S. patent application 20150343196 (Vasapollo, Dec. 3, 2015, “Dual-Purpose Sleep-Wearable Headgear for Monitoring and Stimulating the Brain of a Sleeping Person”) and U.S. Pat. No. 9,320,885 (Vasapollo, Apr. 26, 2016, “Dual-Purpose Sleep-Wearable Headgear for Monitoring and Stimulating the Brain of a Sleeping Person”) disclose headgear for monitoring and stimulating a sleeping person's brain.

U.S. patent application 20140257448 (Arle et al., Sep. 11, 2014, “Head Worn Brain Stimulation Device and Method”) and U.S. Pat. No. 8,909,344 (Arle et al., Dec. 9, 2014, “Head Worn Brain Stimulation Device and Method”) disclose a portable head worn device and methods for brain. U.S. patent application 20180221620 (Metzger, Aug. 9, 2018, “Modulation of Brainwave Activity Using Non-Invasive Stimulation of Sensory Pathways”) discloses an apparatus for modulation of the central nervous system. U.S. patent application 20170165485 (Sullivan et al., Jun. 15, 2017, “Systems and Methods for Non-Invasive Treatment of Head Pain”) discloses systems and methods for non-invasive management of head pain. U.S. patent application 20190381314 (Howard, Dec. 19, 2019, “Transcutaneous Electrically Amplified Cognitive Enhancement System”) discloses devices for transcutaneous nerve stimulation.

U.S. Pat. No. 9,440,070 (Goldwasser et al., Sep. 13, 2016, “Wearable transdermal electrical stimulation devices and methods of using them”) discloses devices and methods for transdermal electrical stimulation. U.S. patent application 20180333576 (Rigaux, Nov. 22, 2018, “External Trigeminal Nerve Stimulation for the Acute Non-Invasive Treatment of Migraine Attacks”) discloses a system and method for non-invasive migraine treatment. U.S. patent application 20180345006 (Ambrose, Dec. 6, 2018, “Electrode Apparatus”) discloses an electrode apparatus for non-invasively applying electrical stimulation to a body. U.S. patent application 20200060571 (Dauguet et al., Feb. 27, 2020, “Device for Measuring and/or Stimulating Brain Activity”) discloses a device for measuring and/or stimulating brain activity.

U.S. patent application 20190001133 (Onarheim et al., Jan. 3, 2019, “Transcranial Electrical Stimulation Device Having Multipurpose Electrodes”) discloses an electrotherapy stimulation device with programmable multipurpose electrodes. U.S. patent applications 20180125689 (Perez et al., May 10, 2018, “Systems and Methods for Using Transcutaneous Electrical Stimulation to Enable Dietary Interventions”) and 20180078754 (Perez et al., Mar. 22, 2018, “Systems and Methods for Using Transcutaneous Electrical Stimulation to Enable Dietary Interventions”) disclose an electrical stimulation system which helps patients with diet compliance.

### SUMMARY OF THE INVENTION

Despite the considerable innovation in the prior art concerning wearable devices for neurostimulation during the past several years, some challenges remain. For example, most current devices (no pun intended) require direct contact with a portion of a person's head which is not covered by hair. This makes such devices relatively obtrusive for wear during daily life and restricts the head locations from which neurostimulation can be applied. Also, the electrodes of many current devices are not sufficiently soft and/or deformable to be comfortable for long-term use. The head-worn neurostimulation devices disclosed herein help to address these challenges.

The head-worn neurostimulation devices disclosed herein can be embodied in a non-invasive wearable neurostimulation device with a plurality of electromagnetic energy emitters which transmit selected patterns of low-intensity electromagnetic energy into a person's head for applications such as: alleviation of pain; addiction treatment; alleviating headaches; reducing body tremors; reducing seizures; addressing stress and anxiety; treating depression; increasing focus or energy level; reducing hunger; improving sleep; addressing attention-related disorders; and neurological rehabilitation. In an example, such neurostimulation devices can be embodied in a wearable ring and/or band which at least partially encircles a person's head. This ring and/or band can include a plurality of flexible, soft electroconductive prongs and/or teeth which protrude into and/or under the person's hair. This design enables good electromagnetic communication with body tissue, even on areas of a person's head which are covered by hair, without causing discomfort during prolonged and/or daily use.

### INTRODUCTION TO THE FIGURES

FIG. 1 shows MCN electrode locations as background information for examples which follow.

FIG. 2 shows a neurostimulation device with a ring and a frontal partial ring.

FIG. 3 shows a neurostimulation device with a front portion with a downward-facing concavity, a middle portion which curves over an ear, and a rear portion.

FIG. 4 shows a neurostimulation device with upper and lower diverging portions from the temple around the back of a head.

FIG. 5 shows a neurostimulation device with an eyewear frame with an upward arm from the sidepiece to the temple or forehead.

FIG. 6 shows a neurostimulation device with front and rear loops spanning from an ear over the top of a head.

FIG. 7 shows a neurostimulation device with a loop from an ear around a forehead and two loops around the back of a head.

FIG. 8 shows a neurostimulation device with an arcuate ring which rests on an ear.

FIG. 9 shows a neurostimulation device with a ring with prongs or teeth which protrude into hair.

FIG. 10 shows a neurostimulation device with an arcuate ring above an ear.

FIG. 11 shows a neurostimulation device with a loop over the top of a head and a loop around the front and back of an ear.

FIG. 12 shows a neurostimulation device with a portion from an ear to the top of a head, a portion from an ear to the back of a head, and an arcuate link between these portions.

FIG. 13 shows a neurostimulation device with a ring and two loops over the top of a head.

FIG. 14 shows a neurostimulation device which curves around an ear and loop around the back of a head.

FIG. 15 shows a neurostimulation device which curves around an ear and loop around a forehead.

FIG. 16 shows a neurostimulation device which curves around an ear and loop over the top of a head.

FIG. 17 shows a neurostimulation device with a wide saddle-shaped device on top of a head.

FIG. 18 shows a neurostimulation device with a narrow saddle-shaped device on top of a head.

FIG. 19 shows a neurostimulation device with a tiara-shaped or hair-band-shaped device on top of a head.

FIG. 20 shows a neurostimulation device with a loop over the top of a head, a loop around the back of a head, and a front-ear-contacting portion.

FIG. 21 shows a neurostimulation device with a loop over the top of a head and a loop around the back of a head.

FIG. 22 shows a neurostimulation device with loop over the top of a head and a portion which curves around an ear.

FIG. 23 shows a neurostimulation device with an undulating head-circling ring which curves over the top of an ear.

FIG. 24 shows a neurostimulation device with a loop around the back of a head and a portion which curves around an ear.

FIG. 25 shows a neurostimulation device with an eyewear frame with a bifurcated sidepiece between an ear and front piece.

FIG. 26 shows a neurostimulation device with upper and lower loops which diverge from an ear to the back of a head.

FIG. 27 shows a neurostimulation device with a forehead loop, a loop over the top of a head, and a semi-circular portion which curves around an ear.

FIG. 28 shows a neurostimulation device with a forehead loop, a loop over the top of a head, and a quarter-circular portion which curves around an ear.

FIG. 29 shows a neurostimulation device with a loop over the top of a head, a loop around the back of a head, and a portion which curves around an ear.

FIG. 30 shows a neurostimulation device with a loop over the top of a head and a portion which encircles an ear.

FIG. 31 shows a neurostimulation device with an undulating ring around a head which rests on an ear.

FIG. 32 shows a neurostimulation device with an eyewear frame with an upward arcuate protrusion over an ear.

FIG. 33 shows a neurostimulation device with a loop over the upper-rear quadrant of a head and a portion which curves around an ear.

FIG. 34 shows a neurostimulation device with an upper front loop and an upper rear loop.

FIG. 35 shows a neurostimulation device with an upper rear loop, a front ear portion, and a rear ear portion.

FIG. 36 shows a neurostimulation device with an undulating ring around a head and also an upper rear loop.

FIG. 37 shows a neurostimulation device with an eyewear frame with an upward undulation on a sidepiece.

FIG. 38 shows a neurostimulation device with bi-level undulating ring and a loop over the top of a head.

FIG. 39 shows a neurostimulation device with an eyewear frame with a protrusion which extends rearward from an ear.

FIG. 40 shows a neurostimulation device with a ring with a concave undulation over an ear.

FIG. 41 shows a neurostimulation device with an undulating ring which rests on an ear.

FIG. 42 shows a neurostimulation device with front and rear loops from an ear over the top of a head.

FIG. 43 shows a neurostimulation device with a portion which loops over a head, a portion which loops around the back of a head, and an arcuate link between them.

FIG. 44 shows a neurostimulation device with an eyewear frame with a concave bifurcation along the sidepiece.

FIG. 45 shows a neurostimulation device with a front loop which spans a forehead and an upper loop over the top of a head.

FIG. 46 shows a neurostimulation device with an eyewear frame with an undulating sidepiece.

FIG. 47 shows a neurostimulation device with an undulating ring and a loop over the top of a head.

FIG. 48 shows a neurostimulation device with an eyewear frame with an upward and rearward protrusion from the top of an ear.

FIG. 49 shows a neurostimulation device with an eyewear frame with an upward loop from the top of an ear.

FIG. 50 shows a neurostimulation device with a tiara shaped or hairband shaped device which loops from an ear over the top of a head.

FIG. 51 shows a neurostimulation device with an eyewear frame with an upward and rearward loop from the top of an ear.

FIG. 52 shows a neurostimulation device with an angled (non-horizontal) ring and a loop around a forehead.

FIG. 53 shows a neurostimulation device with a loop around the back of a head and a portion which curves around an ear.

FIG. 54 shows a neurostimulation device with a ring around a head and two loops over the upper-rear portion of a head.

FIG. 55 shows a neurostimulation device with an eyewear frame with a concave sidepiece.

FIG. 56 shows a neurostimulation device with an eyewear frame which curves around both the front and back of an ear.

FIG. 57 shows a neurostimulation device with an upper branch and a lower branch which diverge as they loop from an ear around the back of a head.

FIG. 58 shows a neurostimulation device with an eyewear frame with a loop over the top of a head.

FIG. 59 shows a neurostimulation device with a loop over the frontal top of a head and a portion which encircles an ear.

FIG. 60 shows a neurostimulation device with an eyewear frame with a sidepiece whose mid-section curves upward.

FIG. 61 shows a neurostimulation device with an eyewear frame whose sidepiece has an upward protrusion from its mid-section.

FIG. 62 shows a neurostimulation device with an arcuate ring around a head, wherein the front portion is higher than the rear portion.

FIG. 63 shows a neurostimulation device with a ring around a head and a central loop over the top of a head.

FIG. 64 shows a neurostimulation device with two-bifurcated loops from an ear over the top of a head.

FIG. 65 shows a neurostimulation device with an eyewear frame with an upper-rear loop over the top of a head.

FIG. 66 shows a neurostimulation device with circular or elliptical ring around a head and a central loop over the top of the head.

FIG. 67 shows a neurostimulation device with two loops which diverge as they extend from an ear to the top and the back of a head.

FIG. 68 shows a neurostimulation device with an eyewear frame with a sidepiece with an upward arm on its mid-section.

FIG. 69 shows a neurostimulation device with a first device with an undulating loop spanning from an ear over the top of a head.

FIG. 70 shows a neurostimulation device with a second device with an undulating loop spanning from an ear over the top of a head.

FIG. 71 shows a neurostimulation device with a third device with an undulating loop spanning from an ear over the top of a head.

FIG. 72 shows a neurostimulation device with a fourth device with an undulating loop spanning from an ear over the top of a head.

FIG. 73 shows a neurostimulation device with flexible prongs and/or teeth which slide into and/or under a hair.

FIG. 74 shows a neurostimulation device with an eyewear frame with flexible prongs and/or teeth which slide into and/or under a hair.

FIG. 75 shows a neurostimulation device with a first device with a ring and an ear-contacting and/or ear-covering portion.

FIG. 76 shows a neurostimulation device with a second device with a ring and an ear-contacting and/or ear-covering portion.

### DETAILED DESCRIPTION OF THE FIGURES

This invention is a non-invasive wearable neurostimulation device which transmits electromagnetic energy into a person's head. There are numerous potential applications for such a neurostimulation device including: alleviation of pain; addiction treatment; alleviating headaches; reducing body tremors; reducing seizures; addressing stress and anxiety; treating depression; increasing focus or energy level; reducing hunger; improving sleep; addressing attention-related disorders; and neurological rehabilitation.

In an example, a neurostimulation device can transmit electromagnetic energy into a person's body in order to modify the electromagnetic activity of their brain. In an example, electromagnetic energy emitters can transmit electromagnetic energy into body tissue in order to modify, adjust, stimulate, and/or selectively block electromagnetic brain activity. In an example, a neurostimulation device can provide anodal stimulation to excite electromagnetic brain activity. In an example, a neurostimulation device can provide cathodal stimulation to inhibit electromagnetic brain activity. In an example, a neurostimulation device can be configured to transmit electromagnetic energy into a particular area of a person's brain. In an example, a neurostimulation device can transmit electromagnetic energy into a person's body in order to modify the electromagnetic activity of other portions of their nervous system. In an example, a neurostimulation device can be configured to transmit electromagnetic energy into a selected nerve.

In an example, electromagnetic energy which is emitted from an electromagnetic energy emitter can be direct current. In an example a neurostimulation device can provide Transcranial Direct Current Stimulation (tDCS). In an example a neurostimulation device can provide Transcranial Electric Stimulation (tES). In an example, electromagnetic energy which is emitted from an electromagnetic energy emitter can be alternating current. In an example a neurostimulation device can provide Transcranial Alternating Current Stimulation (tACS). In an example, electromagnetic energy which is emitted from an electromagnetic energy emitter can vary between alternating current and direct current or provide a combination of them at the same time. In an example a neurostimulation device can provide Transcranial Magnetic Stimulation (tMS).

In an example, electromagnetic energy from an electromagnetic energy emitter can have a particular wave form or wave morphology. In an example, electromagnetic energy from an electromagnetic energy emitter can have a sinusoidal wave pattern. In an example, electromagnetic energy from an electromagnetic energy emitter can have a saw tooth wave, square wave, or triangle wave pattern. In an example, electromagnetic energy from an electromagnetic energy emitter can have a biphasic pattern or tri-phasic pattern.

In an example, electromagnetic energy from an electromagnetic energy emitter can comprise signal spikes. In an example, electromagnetic energy from an electromagnetic energy emitter can have pattern randomization or pattern repetition. In an example, electromagnetic energy from an electromagnetic energy emitter can have a selected Fourier transformation or inverse Fourier transformation pattern. In an example, electromagnetic energy from an electromagnetic energy emitter can replicate (imitate) a natural neural transmission signal or be the inverse of a natural neural transmission signal. In an example, electromagnetic energy from an electromagnetic energy emitter can have a selected signal continuity and/or duty cycle. In an example, electromagnetic energy from an electromagnetic energy emitter can have selected signal cycling times. In an example, electromagnetic energy from an electromagnetic energy emitter can have selected signal ramping and/or signal dampening.

In an example, a neurostimulation device can be specified based on the geometry of the device, including the configurations/shapes of different arms/portions of the device. Additionally, a neurostimulation device can be specified based on the locations of electrodes (e.g. electromagnetic energy transmitters) which are held on the person's head. Electrode locations can be specified using the internationally-recognized Modified Combinatorial Nomenclature (MCN) electrode placement system.

In an example, a neurostimulation device can be embodied in a soft ring and/or band (such as headband) which is worn on a person's head. In an example, a neurostimulation device can comprise a wearable ring and/or band which encircles a person's head at an anterior acute angle in the range of 0 to 45 degrees with respect to a horizontal plane (when the person's head is upright) and a plurality of electromagnetic energy emitters on the ring and/or band. In an example, a neurostimulation device can be embodied in eyewear which is worn on a person's head. In an example, eyewear can have a flexible and/or elastic rear loop which loops around the rear of a person's head.

In an example, electromagnetic energy emitters of this neurostimulation device can be located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz. The electromagnetic energy emitters can be made from a silicone material (such as PDMS) which has been impregnated, doped, coated, or embedded with conductive material in order to provide consistent but comfortable contact with the person's head. In an example, this neurostimulation device can further comprise prongs and/or teeth which protrude from the ring and/or band into and/or under the person's hair. In an example, the prongs and/or teeth can be made from a silicone material (such as PDMS) which has been impregnated, doped, coated, or embedded with conductive material (such as metal and/or carbon) in order to provide consistent but comfortable contact with the person's head.

In an example, a neurostimulation device can be embodied in eyewear which is worn on a person's head to measure electromagnetic brain activity. In an example, the eyewear can include a flexible and/or elastic rear loop which loops around the rear of a person's head to hold one or more electromagnetic energy emitters against the rear of the person's head. In an example, this neurostimulation device can further comprise prongs and/or teeth which protrude into and/or under the person's hair. This can enable good electromagnetic communication with the person's head at locations on the person's head which are covered by hair. In an example, the prongs and/or teeth can be made from a silicone material (such as PDMS) which has been impregnated, doped, coated, or embedded with conductive material (such as a metal and/or carbon) in order to provide consistent but comfortable contact with the person's head. In an example, conductive metal can be silver or aluminum. In an example, carbon material can be in the form of nanotubes or graphene. In an example, electromagnetic energy emitters can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz.

In an example, a neurostimulation device can comprise: a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy emitters are made from an elastic polymer.

In an example, a neurostimulation device can comprise: a wearable ring and/or band which is configured to encircle a person's head at an anterior acute angle in the range of 0 to 45 degrees with respect to a horizontal plane when the person's head is upright; and a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy emitters are made from a polymer which has been impregnated, doped, coated, or embedded with conductive material; and wherein one or more of the electromagnetic energy emitters have prongs and/or teeth which protrude into and/or under the person's hair.

In an example, a neurostimulation device can comprise: a wearable ring and/or band which is configured to encircle a person's head at an anterior acute angle in the range of 0 to 45 degrees with respect to a horizontal plane when the person's head is upright; and a plurality of electromagnetic energy emitters on the band; wherein one or more of the electromagnetic energy emitters are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy emitters are made from a silicone material which has been impregnated, doped, coated, or embedded with silver or carbon; and wherein one or more of the electromagnetic energy emitters have prongs and/or teeth which protrude into and/or under the person's hair.

In an example, an electromagnetic energy emitter can be made by printing high-conductivity ink onto a low-conductivity textile or fabric. In an example, an electromagnetic energy emitter can be made by printing a conductive elastomeric material onto a low-conductivity textile or fabric. In an example, an electromagnetic energy emitter can be made by melting or adhering elastomeric conductive material onto a low-conductivity textile or fabric. In an example, an electromagnetic energy emitter can be made by embroidering conductive material onto a low-conductivity textile or fabric. In an example, an electromagnetic energy emitter can be made by weaving, knitting, sewing, embroidering, layering, laminating, adhering, melting, fusing, printing, spraying, painting, or pressing electroconductive material into (or onto) a fabric or textile. In an example, electroconductive threads, fibers, yarns, strands, filaments, traces, and/or layers within a fabric or textile can be configured near a person's skin in order to receive electromagnetic energy.

In an example, an electromagnetic energy emitter can be attached to a neurostimulation device using an attachment mechanism selected from the group consisting of: adhesive, band, buckle, button, channel, clasp, clip, electronic connector, flexible channel, hook, hook-and-eye mechanism, magnet, pin, plug, pocket, rivet, sewing, snap, tape, tie, and zipper. In an example, an electromagnetic energy emitter can be attached to a neurostimulation device by printing, laminating, adhering, embroidering, melting, and/or sewing electroconductive material. In an example, an electronically-functional fabric or textile with electromagnetic energy emitters can be created by weaving, knitting, sewing, embroidering, layering, laminating, adhering, melting, fusing, printing, spraying, painting, or pressing together electroconductive threads, fibers, yarns, strands, filaments, traces, and/or layers. In an example, electroconductive threads, yarns, fibers, strands, channels, and/or traces comprising electromagnetic energy emitters can have shapes or configurations which are selected from the group consisting of: circular, elliptical, or other conic section; square, rectangular, hexagon, or other polygon; parallel; perpendicular; crisscrossed; nested; concentric; sinusoidal; undulating; zigzagged; and radial spokes.

In an example, an electronically-functional fabric or textile with electromagnetic energy emitters can be created by printing, spraying, or otherwise depositing electroconductive ink or resin onto an otherwise non-conductive fabric or textile. In an example, an electronically-functional circuit with electromagnetic energy emitters can be created as part of a neurostimulation device by printing a conductive pattern with electroconductive ink or resin. In an example, an electronically-functional fabric or textile with electromagnetic energy emitters can be created by laminating electro-conductive members onto a non-conductive substrate. In an example, an electronically-functional fabric or textile with electromagnetic energy emitters can be created by embroidering a generally non-conductive fabric or textile member with electro-conductive members. In an example, an electronically-functional circuit with electromagnetic energy emitters can be created for a neurostimulation device by embroidering a conductive pattern with electroconductive thread.

In an example, an electromagnetic energy emitter can be made with a low-conductivity material selected from the group consisting of: acetate, acrylic, cotton, denim, elastane, latex, linen, Lycra™, neoprene, nylon, nylon, polyester, wool, silicone, polydimethylsiloxane (PDMS), silk, spandex, and rayon. In an example, an electromagnetic energy emitter can be made with a high-conductivity material selected from the group consisting of: aluminum or aluminum alloy; carbon nanotubes, graphene, or other carbon-based material; copper or copper alloy; gold; nickel; silver; and steel. In an example, an electromagnetic energy emitter can be made with polydimethylsiloxane (PDMS) which has been doped or impregnated with aluminum, carbon (in one or more various configurations and formulations), copper, gold, nickel, silver, or steel. In an example, an electromagnetic energy emitter can be made from polydimethylsiloxane (PDMS) and carbon nanotubes. In an example, an electromagnetic energy emitter can be made from polydimethylsiloxane (PDMS) and silver.

In an example, a neurostimulation device can comprise: a flexible arcuate ring and/or band which is configured to be worn around a person's head, wherein a front portion of the ring and/or band loops around the person's forehead, and wherein a rear portion of the ring and/or band loops around the rear of the person's head; a plurality of electromagnetic energy emitters which measure electromagnetic brain activity, wherein a first subset of the electromagnetic energy emitters are located on the front portion of the ring or band, and wherein a second subset of the electromagnetic energy emitters are located on the rear portion of the ring or band; and a plurality of prongs and/or teeth which protrude into and/or under the person's hair. In an example, the prongs and/or teeth can facilitate electromagnetic communication between electromagnetic energy emitters in the second subset of electromagnetic energy emitters and the brain.

In an example, a neurostimulation device can comprise: a flexible arcuate ring and/or band which is configured to be worn around a person's head, wherein a front portion of the ring and/or band loops around the person's forehead, and wherein a rear portion of the ring and/or band loops around the rear of the person's head; a flexible loop which loops over the top of the person's head; a plurality of electromagnetic energy emitters which measure electromagnetic brain activity, wherein a first subset of the electromagnetic energy emitters are located on the front portion of the ring or band, wherein a second subset of the electromagnetic energy emitters are located on the rear portion of the ring or band, and wherein a third subset of the electromagnetic energy emitters are located on the flexible loop; and a plurality of prongs and/or teeth which protrude into and/or under the person's hair. In an example, the prongs and/or teeth can facilitate electromagnetic communication between electromagnetic energy emitters in the second and third subsets of electromagnetic energy emitters and the person's brain.

In an example, a neurostimulation device can comprise: a flexible arcuate ring and/or band which is configured to be worn around a person's head, wherein a front portion of the ring and/or band loops around the person's forehead, and wherein a rear portion of the ring and/or band loops around the rear of the person's head; an ear portion which curves around (the front and/or rear of) the person's ear; a plurality of electromagnetic energy emitters which measure electromagnetic brain activity, wherein a first subset of the electromagnetic energy emitters are located on the front portion of the ring or band, wherein a second subset of the electromagnetic energy emitters are located on the rear portion of the ring or band, and wherein a third subset of the electromagnetic energy emitters are located on the ear portion; and a plurality of prongs and/or teeth which protrude into and/or under the person's hair. In an example, the prongs and/or teeth facilitate electromagnetic communication between electromagnetic energy emitters in the second and third subsets of electromagnetic energy emitters and the person's brain.

In an example, prongs and/or teeth can be made from an elastomeric polymer which is doped, embedded, impregnated, or coated with conductive material. In an example, portions of electromagnetic energy emitters can be made from an elastomeric polymer which is doped, embedded, impregnated, or coated with conductive material. In an example, the polymer can be a silicone material. In an example, the polymer can be PDMS. In an example, the conductive material can be silver. In an example, the conductive material can be carbon. In an example, the conductive material can be carbon nanotubes or graphene.

In an example, an electromagnetic energy emitter can be made from soft deformable conductive material which provides conformal contact with a person's head without creating irritating or painful points of high-pressure contact. In an example, an electromagnetic energy emitter can be made with conductive polydimethylsiloxane (PDMS), thermoplastic polyurethane (TPU), or poly(3,4-ethylenedioxythiophene):poly(styrene sulfonate) (PEDOT:PSS).

In an example, an electromagnetic energy emitter can be made from elastic, stretchable, and/or deformable conductive material. In an example, an electromagnetic energy emitter can be made from elastic, stretchable, and/or deformable conductive material with a Shore A value which is less than 80. In an example, a first set of electromagnetic energy emitters (which are configured to be worn on a portion of a person's head which is not covered by hair) can have a lower average Shore value than a second set of electromagnetic energy emitters (which is configured to be worn on a portion of the person's head which is covered with hair). In an example, emitters which are configured to be worn on portions of a person's head which are covered with hair can comprise a plurality of hair-penetrating and/or hair-engaging protrusions, combs, teeth, or prongs which penetrate and/or engage layers of the person's hair.

In an example, a neurostimulation device can hold electromagnetic energy emitters in electromagnetic communication with a person's frontal lobe. In an example, a portion of a neurostimulation device can hold electromagnetic energy emitters on a person's forehead. In an example, a neurostimulation device can span a person's forehead. In an example, a neurostimulation device which spans a person's forehead can hold a first EEG emitter above a person's right eye and a second EEG emitter above a person's left eye. In an example, electromagnetic energy emitters which are configured to be worn on a person's forehead can have a low Shore A value (e.g. less than 80) so that they conform to the surface shape of the person's forehead for good electromagnetic communication with the person's frontal lobe. In an example, a portion of a neurostimulation device which spans a person's forehead can be transparent or translucent. In an example, a neurostimulation device can be a headband which encircles a person's head in a generally-horizontal manner, holding electromagnetic energy emitters in electromagnetic communication with both the person's frontal lobe and the person's occipital lobe.

In an example, a neurostimulation device can hold electromagnetic energy emitters in electromagnetic communication with a person's occipital lobe. In an example, a neurostimulation device can have a (semicircular) rear loop which curves around a portion of the rear half of a person's head. In an example, this loop can have a plurality of electromagnetic energy emitters which are in electromagnetic communication with the person's occipital lobe. In an example, a neurostimulation device can hold electromagnetic energy emitters in electromagnetic communication with a posterior portion of a head.

In an example, electromagnetic energy emitters which are configured to be worn on the posterior portion of a person's head can have a plurality of hair-penetrating and/or hair-engaging protrusions, combs, teeth, or prongs for good electromagnetic communication with the person's occipital lobe. These protrusions, combs, teeth, or prongs can protrude between strands of hair to contact the person's skin. In an example, electromagnetic energy emitters which are worn on the posterior a person's head can have a plurality of hair-engaging protrusions, combs, teeth, or prongs which hold the emitters in place.

In an example, a wearable mobile neurostimulation device for monitoring electromagnetic brain activity can include an array of hair-penetrating conductive protrusions with a two-dimensional cross-sectional area in a plane which is substantially parallel to the surface of a person's head when the neurostimulation device is worn. In an example, protrusions toward the center of this cross-sectional area can be farther apart than protrusions toward the periphery of this cross-sectional area. In an example, protrusions toward the center of this cross-sectional area can be shorter than protrusions toward the periphery of this cross-sectional area. In an example, protrusions toward the center of this cross-sectional area can have a lower Shore A value than protrusions toward the periphery of this cross-sectional area. In an example, protrusions toward the center of this cross-sectional area can be less conductive than protrusions toward the periphery of this cross-sectional area.

In an example, a neurostimulation device can include a rubber cushion which gently holds an electromagnetic energy emitter against a person's head. In an example, a neurostimulation device can include a gel layer which gently holds an electromagnetic energy emitter against a person's head. In an example, a neurostimulation device can include compressive foam which gently holds an electromagnetic energy emitter against a person's head. In an example, a neurostimulation device can have a silicone-based layer which gently holds an electromagnetic energy emitter against a person's head. In an example, an electromagnetic energy emitter can be made with natural or synthetic sponge material. In an example, an electromagnetic energy emitter can be made with polydimethylsiloxane (PDMS), thermoplastic polyurethane (TPU), or poly(3,4-ethylenedioxythiophene):poly(styrene sulfonate) (PEDOT:PSS).

In an example, each electromagnetic energy emitter in a plurality of electromagnetic energy emitters can have its own rubber cushion, gel layer, or compressive foam which gently holds it against a person's head. In an example, the Shore A values of individual emitter cushions (or other compressive pieces) can be different for different individual electromagnetic energy emitters. In an example, electromagnetic energy emitters which are worn on a person's forehead can have softer cushions (or other compressive pieces) than electromagnetic energy emitters worn elsewhere on the person's head. In an example, a first set of electromagnetic energy emitters can be held against a person's head by a first set of cushions (or other compressive pieces) with a first average Shore A value, and a second set of electromagnetic energy emitters can be held against the person's head by a second set of cushions (or other compressive pieces) with a second average Shore A value, wherein the second average Shore A value is greater than the first average Shore A value.

In an example, a neurostimulation device can comprise: (a) a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and (b) a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy emitters are made from an elastic polymer.

In an example, a neurostimulation device can comprise: (a) a wearable ring and/or band which is configured to encircle a person's head at an anterior acute angle in the range of 0 to 45 degrees with respect to a horizontal plane when the person's head is upright; and (b) a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy emitters are made from a polymer which has been impregnated, doped, coated, or embedded with conductive material; and wherein one or more of the electromagnetic energy emitters have prongs and/or teeth which protrude into and/or under the person's hair. In an example, this device can be worn on the Babylon 5 space station without appearing to be out of the ordinary.

In an example, a neurostimulation device can comprise: (a) a wearable ring and/or band which is configured to encircle a person's head at an anterior acute angle in the range of 0 to 45 degrees with respect to a horizontal plane when the person's head is upright; and (b) a plurality of electromagnetic energy emitters on the band; wherein one or more of the electromagnetic energy emitters are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy emitters are made from a silicone material which has been impregnated, doped, coated, or embedded with silver or carbon; and wherein one or more of the electromagnetic energy emitters have prongs and/or teeth which protrude into and/or under the person's hair.

FIG. 1 shows Modified Combinatorial Nomenclature (MCN) electrode locations on the left side of a person's head. In an example, one or more electromagnetic energy emitters (such as EEG emitters) can be located at one or more of these electrode locations. The figures and examples which follow in this disclosure show the left side of a person's head, but the neurostimulation devices which are specified in these examples can also be laterally symmetric on the right side of the person's head even though it is not shown.

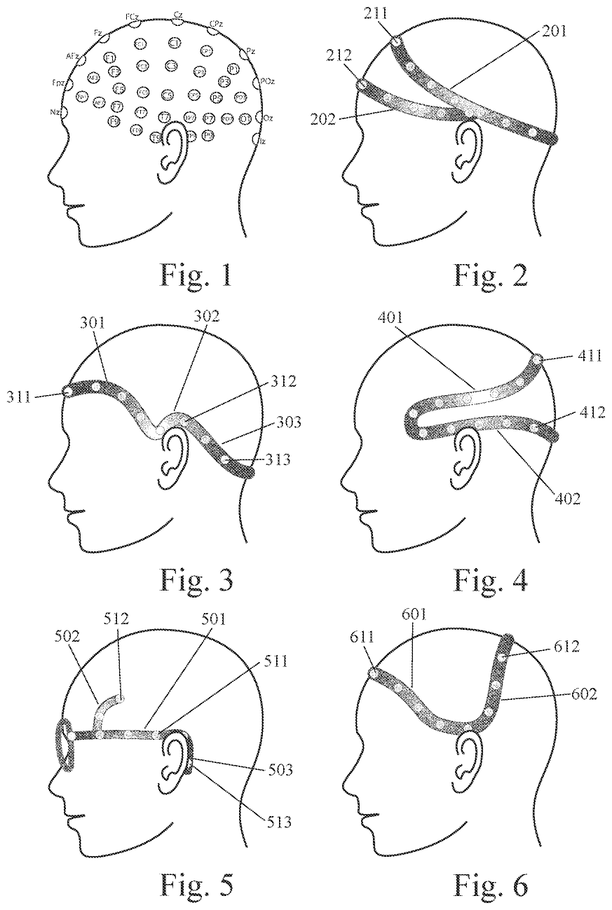

FIG. 2 shows the left side of a neurostimulation device comprising: a ring 201 which encircles a person's head spanning the front and the back of the head and holds at least one first electromagnetic energy emitter 211 (substantially located at an electrode position selected from the group consisting of Iz, PO7, P7, F5, F3, F1, Fz, TP7, FC5, and C5); and a partial ring 202 which partially encircles the person's head (spanning the front of the person's head) and holds at least one second electromagnetic energy emitter 212 (substantially located at an electrode position selected from the group consisting of FP1, FPz, AF7, F7, FT7, and T7).

In an example, a neurostimulation device can comprise: a ring which encircles a person's head spanning the front and the back of the head and holds at least one first electromagnetic energy emitter (substantially located at an electrode position selected from the group consisting of Iz, PO7, P7, F5, F3, F1, Fz, TP7, FC5, and C5); and a partial ring which partially encircles the person's head (spanning the front of the head).

In an example, a neurostimulation device can comprise: a ring which encircles a person's head spanning the front and the back of the person's head; and a partial ring which partially encircles the person's head (spanning the front of the head) and holds at least one second electromagnetic energy emitter (substantially located at an electrode position selected from the group consisting of FP1, FPz, AF7, F7, FT7, and T7).

In an example: a first virtual plane which best fits the ring can intersect a horizontal plane when the person is standing up, forming a first forward-facing angle between 5 and 75 degrees; a second virtual plane which best fits the partial ring can intersect the horizontal plane, forming a second forward-facing angle between 5 and 75 degrees; and the first angle can be at least 5 degrees greater than the second angle. In an example, the intersection of the ring and the partial ring can form a forward-facing angle between 5 and 65 degrees. In an example, the front of the ring can be higher than the rear of the ring. In an example, a frontal portion of the partial ring can be lower than the ring. In an example, the partial ring can span the person's forehead. In an example, the partial ring can intersect and/or join the ring: at a location (directly) above the person's ear; and/or at a location within 2″ of the front-to-back midpoint of the side of the person's head. In an example, the ring and/or the partial ring can have an (upward-facing) concavity. In an example, the ring can be more upwardly-concave than the partial ring. In an example, the ring can be less upwardly-concave than partial ring. In an example, the ring can be circular, oval, or elliptical. In an example, the partial ring can be a semicircle, a semi-oval, or a semi-ellipse. In an example, this neurostimulation device can be a bifurcating ring around a person's head, wherein the ring bifurcates as it spans the front of the person's head. In an example, this neurostimulation device can comprise a headband.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 3 shows the left side of a neurostimulation device comprising: a front portion 301 which spans from a person's ear to their forehead and holds at least one first electromagnetic energy emitter 311 (substantially located at an electrode position selected from the group consisting of FPz, AF3, F5, and FT7), wherein the front portion has a downward-facing concavity; a middle portion 302 which is connected to the front portion, curves over the top of the person's outer ear, and holds at least one second electromagnetic energy emitter 312 (substantially located at an electrode position selected from the group consisting of TP7, T9, and T7); and a rear portion 303 which is connected to the middle portion, spans the rear of the person's head, and holds at least one third electromagnetic energy emitter 313 (substantially located at an electrode position selected from the group consisting of P9 and TP9).

In an example, the left side of a neurostimulation device can comprise: a front portion which spans from a person's ear to their forehead and holds at least one first electromagnetic energy emitter (substantially located at an electrode position selected from the group consisting of FPz, AF3, F5, and FT7), wherein the front portion has a downward-facing concavity; a middle portion which is connected to the front portion and curves over the top of the person's outer ear; and a rear portion which is connected to the middle portion and spans the rear of the person's head.

In an example, the left side of a neurostimulation device can comprise: a front portion which spans from a person's ear to their forehead and has a downward-facing concavity; a middle portion which is connected to the front portion, curves over the top of the person's outer ear, and holds at least one second electromagnetic energy emitter (substantially located at an electrode position selected from the group consisting of TP7, T9, and T7); a rear portion which is connected to the middle portion and spans the rear of the person's head.

In an example, the left side of a neurostimulation device can comprise: a front portion which spans from a person's ear to their forehead and has a downward-facing concavity; a middle portion which is connected to the front portion and curves over the top of the person's outer ear; a rear portion which is connected to the middle portion, spans the rear of the person's head, and holds at least one third electromagnetic energy emitter (substantially located at an electrode position selected from the group consisting of P9 and TP9).

In an example, this neurostimulation device can comprise an undulating ring which encircles a person's head, spanning their forehead and the rear of their head, with upward undulations over the tops of the person's ears. In an example, this neurostimulation device can comprise an undulating ring which encircles a person's head, spanning their forehead and the rear of their head, with sinusoidal waves over the tops of the person's ears. In an example, a virtual plane which best fits this undulating ring can intersect a horizontal plane when the person is standing up, forming a forward-facing angle between 5 and 75 degrees. In an example, the front of this undulating ring can be higher than the rear of this undulating ring. In an example, the rear of the undulating ring can be lower than the top of the person's ear. In an example, this neurostimulation device can comprise a headband.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 4 shows the left side of a neurostimulation device comprising: an upper arm/portion 401 which spans from an area within 2″ of a person's temple to the back of their head and holds at least one first electromagnetic energy emitter 411 (substantially located at an electrode position selected from the group consisting of P3, P1, Pz, F9, F7, CP5, FC5, and C5); and a lower arm/portion 402 which spans from the area within 2″ of a person's temple to the back of their head and holds at least one second electromagnetic energy emitter 412 (substantially located at an electrode position selected from the group consisting of Iz, O1, Oz, PO7, P7, F9, TP7, FT9, and T7), wherein the front of the upper arm/portion is connected to the front of the lower arm/portion, and wherein the rear of the upper arm/portion is higher than the rear of the lower arm/portion.

In an example, the left side of a neurostimulation device can comprise: an upper arm/portion which spans from an area within 2″ of a person's temple to the back of their head and holds at least one first electromagnetic energy emitter (substantially located at an electrode position selected from the group consisting of P3, P1, Pz, F9, F7, CP5, FC5, and C5); and a lower arm/portion which spans from the area within 2″ of a person's temple to the back of their head, wherein the front of the upper arm/portion is connected to the front of the lower arm/portion, and wherein the rear of the upper arm/portion is higher than the rear of the lower arm/portion.

In an example, the left side of a neurostimulation device can comprise: an upper arm/portion which spans from an area within 2″ of a person's temple to the back of their head; and a lower arm/portion which spans from the area within 2″ of a person's temple to the back of their head and holds at least one second electromagnetic energy emitter (substantially located at an electrode position selected from the group consisting of Iz, O1, Oz, PO7, P7, F9, TP7, FT9, and T7), wherein the front of the upper arm/portion is connected to the front of the lower arm/portion, and wherein the rear of the upper arm/portion is higher than the rear of the lower arm/portion.

In an example, the upper arm/portion can have an upward-facing concavity and the lower arm/portion can have a downward-facing concavity. In an example, the neurostimulation device can span approximately two-thirds of the front-to-back width of the side of the person's head. In an example, the lower arms/portions of the neurostimulation device can rest on top the person's ears. In an example, the neurostimulation device overall can have a rear-facing concavity. In an example, the left-side-portion of the neurostimulation device can have the shape of a rear-opening “bobby pin.”

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 5 shows the left side of a neurostimulation device comprising: a relatively-conventional eyeglass frame 501 which holds at least one first electromagnetic energy emitter 511 (substantially located at an electrode position selected from the group consisting of Nz, F9, FT9, FT7, and T7); an arcuate upward arm 502 which extends upward from the side of the eyeglass frame to the person's temple and/or forehead and holds at least one second electromagnetic energy emitter 512 (substantially located at an electrode position selected from the group consisting of AF7 and F5); and a rear arm 503 which curves around the rear of the person's ear and holds at least one third electromagnetic energy emitter 513 (substantially located at an electrode position selected from the group consisting of TP9 and TP7).

In an example, a neurostimulation device can comprise: a relatively-conventional eyeglass frame which holds at least one first electromagnetic energy emitter (substantially located at an electrode position selected from the group consisting of Nz, F9, FT9, FT7, and T7); an arcuate upward arm which extends upward from the side of the eyeglass frame to the person's temple and/or forehead; and a rear arm which curves around the rear of the person's ear.

In an example, a neurostimulation device can comprise: a relatively-conventional eyeglass frame; an arcuate upward arm which extends upward from the side of the eyeglass frame to the person's temple and/or forehead and holds at least one second electromagnetic energy emitter (substantially located at an electrode position selected from the group consisting of AF7 and F5); and a rear arm which curves around the rear of the person's ear.

In an example, a neurostimulation device can comprise: a relatively-conventional eyeglass frame; an arcuate upward arm which extends upward from the side of the eyeglass frame to the person's temple and/or forehead; and a rear arm which curves around the rear of the person's ear and holds at least one third electromagnetic energy emitter (substantially located at an electrode position selected from the group consisting of TP9 and TP7).

In an example, the arcuate upward arm can have a rear-facing concavity. In an example, the arcuate upward arm can have a forward-facing concavity. In an example, an upward arm can pivot and/or rotate around its connection to the eyeglass frame. In an example, an upward arm can pivot from a first configuration in which it is aligned with and/or hidden behind the eyeglass frame when not in use and a second configuration in which is extends upward from the eyeglass frame for use.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 6 shows the left side of a neurostimulation device comprising: a front arm/portion 601 which spans from an area (directly) above a person's ear to the front of the person's head and holds at least one first electromagnetic energy emitter 611 (substantially located at an electrode position selected from the group consisting of AF3, AFz, F5, FT7, and T7); and a rear arm/portion 602 which spans from the area above the person's ear to the rear of the person's head and holds at least one second electromagnetic energy emitter 612 (substantially located at an electrode position selected from the group consisting of TP7, CP5, CP3, CP1, and CPz).

In an example, the left side of a neurostimulation device can comprise: a front arm/portion which spans from an area above a person's ear around the front of the person's head and holds at least one first electromagnetic energy emitter (substantially located at an electrode position selected from the group consisting of AF3, AFz, F5, FT7, and T7); and a rear arm/portion which spans from the area above the person's ear around the rear of the person's head.

In an example, the left side of a neurostimulation device can comprise: a front arm/portion which spans from an area above a person's ear around the front of the person's head; and a rear arm/portion which spans from the area above the person's ear around the rear of the person's head and holds at least one second electromagnetic energy emitter (substantially located at an electrode position selected from the group consisting of TP7, CP5, CP3, CP1, and CPz).

In an example, the neurostimulation device can be shaped like a saddle on top of the person's head. In an example, the front arm/portion and the rear arm/portion can connect at an area which is directly over the person's ear. In an example, the neurostimulation device can rest on top of the person's ears. In an example, the rear arm/portion can be more vertical than the front arm/portion. In an example, the front arm/portion can span the person's forehead. In an example, the front arm/portion can have a downward-facing concavity.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

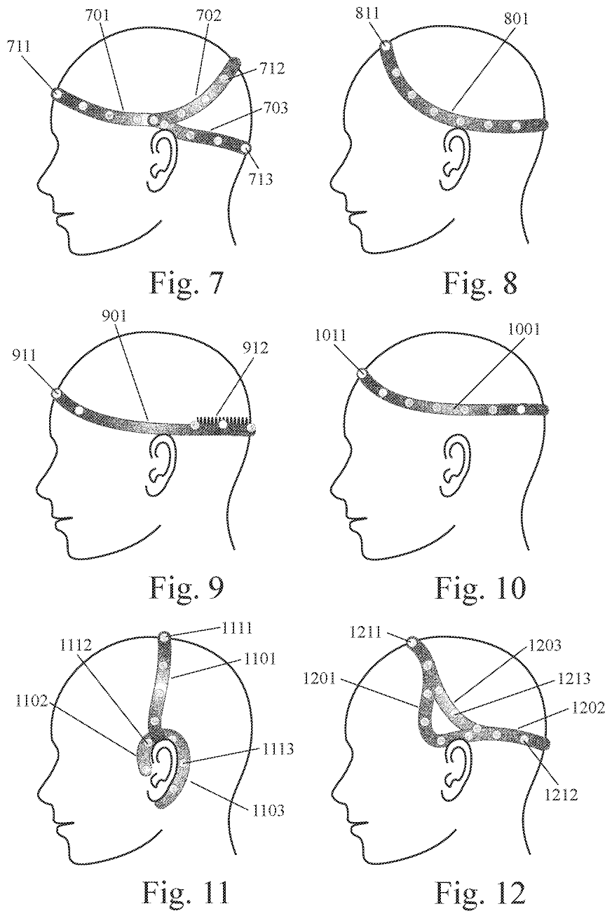

FIG. 7 shows the left side of a neurostimulation device comprising: a first arm/portion 701 which spans from an area (directly) above a person's ear to the person's forehead and holds at least one first electromagnetic energy emitter 711 (substantially located at an electrode position selected from the group consisting of FP1, FPz, AF7, F7, FT7, and T7); a second arm/portion 702 which spans from the area above the person's ear to the rear of the person's head and holds at least one second electromagnetic energy emitter 712 (substantially located at an electrode position selected from the group consisting of P5, P3, P1, Pz, CP5, T7); and a third arm/portion 703 which spans from the area above the person's ear to the rear of the person's head and holds at least one third electromagnetic energy emitter 713 (substantially located at an electrode position selected from the group consisting of Iz, P9, P7, TP9, and TP7), wherein the first, second, and third arms/portions are connected, and wherein the rear of the second arm/portion is higher than the rear of the third arm/portion.

In an example, the left side of a neurostimulation device can comprise: a first arm/portion which spans from an area (directly) above a person's ear to the person's forehead and holds at least one first electromagnetic energy emitter (substantially located at an electrode position selected from the group consisting of FP1, FPz, AF7, F7, FT7, and T7); a second arm/portion which spans from the area above the person's ear to the rear of the person's head; and a third arm/portion which spans from the area above the person's ear to the rear of the person's head, wherein the first, second, and third arms/portions are connected, and wherein the rear of the second arm/portion is higher than the rear of the third arm/portion.

In an example, the left side of a neurostimulation device can comprise: a first arm/portion which spans from an area (directly) above a person's ear to the person's forehead; a second arm/portion which spans from the area above the person's ear to the rear of the person's head and holds at least one second electromagnetic energy emitter (substantially located at an electrode position selected from the group consisting of P5, P3, P1, Pz, CP5, T7); and a third arm/portion which spans from the area above the person's ear to the rear of the person's head, wherein the first, second, and third arms/portions are connected, and wherein the rear of the second arm/portion is higher than the rear of the third arm/portion.

In an example, the left side of a neurostimulation device can comprise: a first arm/portion which spans from an area (directly) above a person's ear to the person's forehead; a second arm/portion which spans from the area above the person's ear to the rear of the person's head; and a third arm/portion which spans from the area above the person's ear to the rear of the person's head and holds at least one third electromagnetic energy emitter (substantially located at an electrode position selected from the group consisting of Iz, P9, P7, TP9, and TP7), wherein the first, second, and third arms/portions are connected, and wherein the rear of the second arm/portion is higher than the rear of the third arm/portion.

In an example, the intersection of the second arm/portion and the third arm/portion can form a rear-facing angle between 5 and 65 degrees. In an example, the front of the first arm/portion can be higher than the rear of the third arm/portion. In an example, the three arms/portions can intersect at a location (directly) above the person's ear and/or at a location within 2″ of the front-to-back midpoint of the side of the person's head. In an example, the first arm/portion can have an upward-facing concavity. In an example, the second arm/portion can have an upwardly-facing concavity. In an example, this neurostimulation device can comprise a bifurcating ring around a person's head, wherein the ring bifurcates as it spans the rear of the person's head. In an example, the first and third arms/portions can comprise a continuous band around a person's head. In an example, this neurostimulation device can comprise a headband.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 8 shows the left side of a neurostimulation device comprising: a ring 801 which encircles a person's head and spans the front and rear of their head, holding at least one electromagnetic energy emitter 811 (substantially located at an electrode position selected from the group consisting of O1, Oz, PO7, P7, F5, F3, F1, Fz, TP7, FT7, FC5, and T7).

In an example, a neurostimulation device can comprise: a ring which encircles a person's head and holds at least one electromagnetic energy emitter 811 (substantially located at an electrode position selected from the group consisting of O1, Oz, PO7, P7, F5, F3, F1, Fz, TP7, FT7, FC5, and T7).

In an example, the front of the ring can be higher than the rear of the ring. In an example, a virtual plane which best fits this ring can intersect a horizontal plane when the person is standing up, forming a forward-facing angle between 5 and 75 degrees. In an example, the ring can have an upward-facing concavity. In an example, the ring can rest on the tops of the person's ears. In an example, the front of the ring can be more vertical than the rear of the ring. In an example, this ring can comprise a headband. In an example, this ring can comprise the perimeter of a skullcap or baseball cap.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 9 shows the left side of a neurostimulation device comprising: a ring and/or band 901 which encircles a person's head; one or more electromagnetic energy emitters 911 which are substantially located at electrode positions selected from the group consisting of O1, Oz, FP1, FPz, PO7, AF7, P7, F7, TP7, FT7, and T7; and prongs or teeth 912 which protrude into and/or under the person's hair at locations on the person's head which are covered by hair. In an example, a neurostimulation device can comprise: a ring and/or band which encircles a person's head; and one or more electromagnetic energy emitters which are substantially located at electrode positions selected from the group consisting of O1, Oz, FP1, FPz, PO7, AF7, P7, F7, TP7, FT7, and T7, wherein one or more of the electromagnetic energy emitters have prongs or teeth which protrude into and/or under a person's hair.

In an example, the front of the ring and/or band can be higher than the rear of the ring and/or band. In an example, a virtual plane which best fits this ring and/or band can intersect a horizontal plane when the person is standing up, forming a forward-facing angle between 5 and 75 degrees. In an example, the front portion of the ring and/or band can have an upward-facing concavity. In an example, the ring and/or band can rest on the tops of the person's ears. In an example, a ring and/or band can comprise a headband. In an example, a ring and/or band can comprise the perimeter of a skullcap or baseball cap.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent.

In an example, a neurostimulation device can comprise: a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and a plurality of electromagnetic energy emitters on the ring and/or band; wherein one or more of the electromagnetic energy emitters are located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz; and wherein one or more of the electromagnetic energy emitters are made from an elastic polymer.

In an example, a neurostimulation device can comprise: a wearable ring and/or band which is configured to encircle a person's head at an anterior acute angle in the range of 0 to 45 degrees with respect to a horizontal plane when the person's head is upright; and a plurality of electromagnetic energy emitters on the ring and/or band; wherein one or more of the electromagnetic energy emitters are located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz; wherein one or more of the electromagnetic energy emitters are made from a silicone material which has been impregnated, doped, coated, or embedded with metal; and wherein one or more of the electromagnetic energy emitters have prongs and/or teeth which protrude into and/or under the person's hair.

In an example, a neurostimulation device can comprise: a wearable ring and/or band which is configured to encircle a person's head at an anterior acute angle in the range of 0 to 45 degrees with respect to a horizontal plane when the person's head is upright; and a plurality of electromagnetic energy emitters on the ring and/or band; wherein one or more of the electromagnetic energy emitters are located at placement sites of Modified Combinatorial Nomenclature (MCN) electrode placement system which are selected from the group consisting of O1, Oz, FP1, FPz, PO7, AF7, P7, F7, TP7, FT7, and T7; wherein one or more of the electromagnetic energy emitters are made from a silicone material which has been impregnated, doped, coated, or embedded with carbon; and wherein one or more of the electromagnetic energy emitters have prongs and/or teeth which protrude into and/or under the person's hair.

In an example, a neurostimulation device can be embodied in a soft ring and/or band (such as headband) which is worn around a person's head to measure electromagnetic brain activity. In an example, a neurostimulation device comprising a wearable ring and/or band which encircles a person's head at an anterior acute angle in the range of 0 to 45 degrees with respect to a horizontal plane (when the person's head is upright) and a plurality of electromagnetic energy emitters on the ring and/or band.

In an example, this neurostimulation device can further comprise prongs and/or teeth which protrude into and/or under the person's hair. This can enable good electromagnetic communication with the person's head at locations on the person's head which are covered by hair. In an example, the prongs and/or teeth can be made from a silicone material (such as PDMS) which has been impregnated, doped, coated, or embedded with conductive material (such as a metal and/or carbon) in order to provide consistent but comfortable contact with the person's head. In an example, conductive metal can be silver or aluminum. In an example, carbon material can be in the form of nanotubes or graphene. In an example, electromagnetic energy emitters can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz. Variations discussed elsewhere in this or priority-linked disclosures can also be applied to this example.

FIG. 10 shows the left side of a neurostimulation device comprising: a ring 1001 which encircles a person's head at a level which is at least 1″ above the person's ears, wherein this ring holds at least one electromagnetic energy emitter 1011 (substantially located at an electrode position selected from the group consisting of PO3, AF3, AFz, P5, F5, CP5, FC5, and C5).

In an example, a neurostimulation device can comprise: a ring which encircles a person's head at a level which is at least 1″ above the person's ears, wherein this ring holds at least one electromagnetic energy emitter (substantially located at an electrode position selected from the group consisting of PO3, AF3, AFz, P5, F5, CP5, FC5, and C5).

In an example, the front of the ring can be higher than the rear of the ring. In an example, a virtual plane which best fits this ring can intersect a horizontal plane when the person is standing up, forming a forward-facing angle between 5 and 75 degrees. In an example, the front portion of the ring can have an upward-facing concavity. In an example, this ring can comprise a headband. In an example, this ring can comprise the perimeter of a skullcap or baseball cap.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 11 shows the left side of a neurostimulation device comprising: a first arm/portion 1101 which spans from an area (directly) above a person's ear to the top of their head and holds at least one first electromagnetic energy emitter 1111 (substantially located at an electrode position selected from the group consisting of T7, C5, C3, C1, and Cz); a second arm/portion 1102 which curves around the front of the person's ear and holds at least one second electromagnetic energy emitter 1112 (substantially located at an electrode position selected from the group consisting of T9, T7); and a third arm/portion 1103 which curves around the rear of the person's ear and holds at least one third electromagnetic energy emitter 1113 (substantially located at an electrode position selected from the group consisting of P9, TP9, and TP7).

In an example, the left side of a neurostimulation device can comprise: a first arm/portion which spans from an area (directly) above a person's ear to the top of their head and holds at least one first electromagnetic energy emitter (substantially located at an electrode position selected from the group consisting of T7, C5, C3, C1, and Cz); a second arm/portion which curves around the front of the person's ear; and a third arm/portion which curves around the rear of the person's ear.

In an example, the left side of a neurostimulation device can comprise: a first arm/portion which spans from an area (directly) above a person's ear to the top of their head; a second arm/portion which curves around the front of the person's ear and holds at least one second electromagnetic energy emitter (substantially located at an electrode position selected from the group consisting of T9, T7); and a third arm/portion which curves around the rear of the person's ear.

In an example, the left side of a neurostimulation device can comprise: a first arm/portion which spans from an area (directly) above a person's ear to the top of their head; a second arm/portion which curves around the front of the person's ear; and a third arm/portion which curves around the rear of the person's ear and holds at least one third electromagnetic energy emitter (substantially located at an electrode position selected from the group consisting of P9, TP9, and TP7).

In an example, this neurostimulation device can comprise a pair of headphones. In an example, the first arm/portion can loop over the top of the person's head. In an example, the second and third arms/portions can collectively span between one half and three-quarters of the circumference around a person's ear. In an example, the first, second, and third arms/portions of this neurostimulation device can intersect and/or be connected to each other at an area (directly) above the person's ear. In an example, the first arm/portion can be substantially vertical. In an example, the first arm/portion can have a forward-facing concavity. In an example, the first arm/portion can have a rear-facing concavity. In an example, the third arm/portion can also hold an electrode which is attached to the person's ear lobe.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 12 shows the left side of a neurostimulation device comprising: a first arm/portion 1201 which spans from a first location within 2″ of a person's ear to the top of the person's head and holds at least one first electromagnetic energy emitter 1211 (substantially located at an electrode position selected from the group consisting of FT7, FC5, FC3, FC1, FCz, and T7); a second arm/portion 1202 which spans from the first location to the rear of the person's head and holds at least one second electromagnetic energy emitter 1212 (substantially located at an electrode position selected from the group consisting of O1, Oz, PO7, P7, TP7, T7), wherein the first arm/portion and the second arm/portion are connected to each other at the first location; and a third arm/portion 1203 which spans between the first arm/portion and the second arm/portion at locations which are different than the first location and which holds at least one third electromagnetic energy emitter 1213 (substantially located at electrode position C5).

In an example, the left side of a neurostimulation device can comprise: a first arm/portion which spans from a first location within 2″ of a person's ear to the top of the person's head and holds at least one first electromagnetic energy emitter (substantially located at an electrode position selected from the group consisting of FT7, FC5, FC3, FC1, FCz, and T7); a second arm/portion which spans from the first location to the rear of the person's head, wherein the first arm/portion and the second arm/portion are connected to each other at the first location; and a third arm/portion which spans between the first arm/portion and the second arm/portion at locations which are different than the first location.

In an example, the left side of a neurostimulation device can comprise: a first arm/portion which spans from a first location within 2″ of a person's ear to the top of the person's head; a second arm/portion which spans from the first location to the rear of the person's head and holds at least one second electromagnetic energy emitter (substantially located at an electrode position selected from the group consisting of O1, Oz, PO7, P7, TP7, T7), wherein the first arm/portion and the second arm/portion are connected to each other at the first location; and a third arm/portion which spans between the first arm/portion and the second arm/portion at locations which are different than the first location.

In an example, the left side of a neurostimulation device can comprise: a first arm/portion which spans from a first location within 2″ of a person's ear to the top of the person's head; a second arm/portion which spans from the first location to the rear of the person's head, wherein the first arm/portion and the second arm/portion are connected to each other at the first location; and a third arm/portion which spans between the first arm/portion and the second arm/portion at locations which are different than the first location and which holds at least one third electromagnetic energy emitter (substantially located at electrode position C5).

In an example, the first arm/portion loops over the top of the person's head. In an example, the second arm/portion loops around the rear of the person's head. In an example, the third arm/portion spans between the left-side mid-point of the first arm/portion and the left-side mid-point of the second arm/portion. In an example, the third arm/portion spans between midsections of the first and second arm/portions. In an example, the third arm/portion connects midsections of the first and second arm/portions. In an example, the first arm/portion can have a forward-facing concavity. In an example, the second arm/portion can have a downward-facing concavity. In an example, the best fitting virtual plane for the first arm/portion and the best fitting virtual plane for the second arm/portion can be substantially perpendicular to each other. In an example, this neurostimulation device can rest on the tops of the person's ears. In an example, a left-side view of this neurostimulation device can have the shape of an arcuate capital letter “A” which has been rotated clockwise between 100 and 170 degrees.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

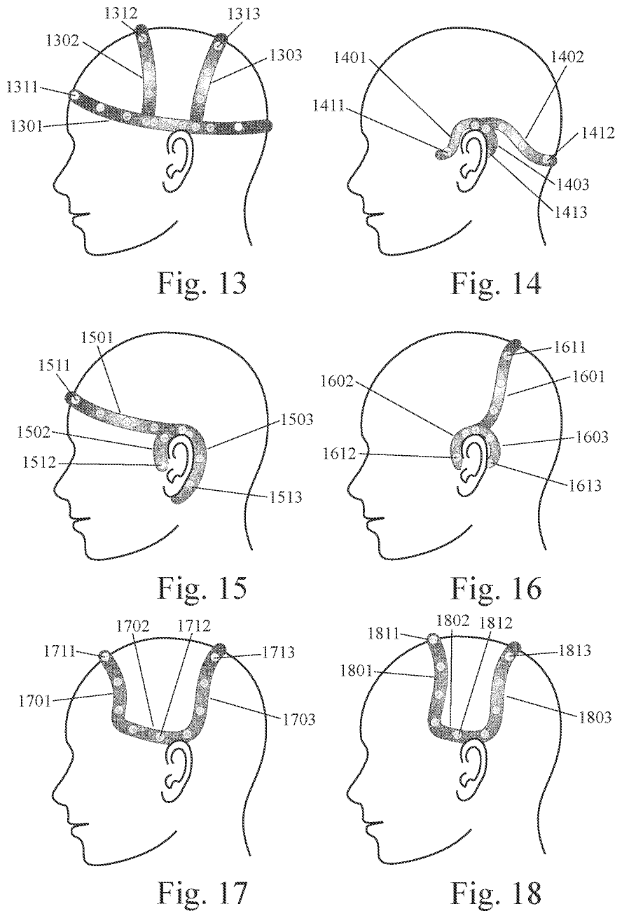

FIG. 13 shows the left side of a neurostimulation device comprising: a ring 1301 which encircles a person's head and holds at least one first electromagnetic energy emitter 1311 (substantially located at an electrode position selected from the group consisting of O1, Oz, FP1, FPz, PO7, AF7, P7, F7, TP7, FT7, and T7); a front-upper arm/portion 1302 which loops upward from the ring over the top of the person's head and holds at least one second electromagnetic energy emitter 1312 (substantially located at an electrode position selected from the group consisting of FT7, FC5, FC3, FC1, and FCz); and a rear-upper arm/portion 1303 which loops upward from the ring over the top of the person's head and holds at least one third electromagnetic energy emitter 1313 (substantially located at an electrode position selected from the group consisting of TP7, CP5, CP3, CP1, and CPz), wherein the front-upper arm/portion is closer to the front of the person's head than the rear-upper arm/portion.

In an example, a neurostimulation device can comprise: a ring which encircles a person's head and holds at least one first electromagnetic energy emitter (substantially located at an electrode position selected from the group consisting of O1, Oz, FP1, FPz, PO7, AF7, P7, F7, TP7, FT7, and T7); a front-upper arm/portion which loops upward from the ring over the top of the person's head; and a rear-upper arm/portion which loops upward from the ring over the top of the person's head, wherein the front-upper arm/portion is closer to the front of the person's head than the rear-upper arm/portion.

In an example, a neurostimulation device can comprise: a ring which encircles a person's head; a front-upper arm/portion which loops upward from the ring over the top of the person's head and holds at least one second electromagnetic energy emitter (substantially located at an electrode position selected from the group consisting of FT7, FC5, FC3, FC1, and FCz); and a rear-upper arm/portion which loops upward from the ring over the top of the person's head, wherein the front-upper arm/portion is closer to the front of the person's head than the rear-upper arm/portion.

In an example, a neurostimulation device can comprise: a ring which encircles a person's head; a front-upper arm/portion which loops upward from the ring over the top of the person's head; and a rear-upper arm/portion which loops upward from the ring over the top of the person's head and holds at least one third electromagnetic energy emitter (substantially located at an electrode position selected from the group consisting of TP7, CP5, CP3, CP1, and CPz), wherein the front-upper arm/portion is closer to the front of the person's head than the rear-upper arm/portion.

In an example, the ring can be tilted so that the front of the ring is higher than the rear of the ring. In an example, the ring can have an upward-facing concavity. In an example, the front-upper arm/portion can have a front-facing concavity. In an example, the rear-upper arm/portion can have a rear-facing concavity. In an example, the ring can rest on the tops of the person's ears. In an example, the front-upper arm/portion and the rear-upper arm/portion can diverge from each other as they leave the ring and loop over the person's head. In an example, the front-upper arm/portion can be anterior to a virtual vertical plane which connects a person's ears and the rear-upper arm/portion can be posterior relative to this virtual vertical plane.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection with the ring. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 14 shows the left side of a neurostimulation device comprising: a first arm/portion 1401 which curves forward and downward from a person's ear and holds at least one first electromagnetic energy emitter 1411 (substantially located at an electrode position selected from the group consisting of T9 and T7); a second arm/portion 1402 which curves backward and downward from the person's ear and holds at least one second electromagnetic energy emitter 1412 (substantially located at an electrode position selected from the group consisting of Iz, P7, TP7); and a third arm/portion 1403 which loops around the rear of the person's head and holds at least one third electromagnetic energy emitter 1413 (substantially located at an electrode position selected from the group consisting of TP9 and TP7).

In an example, the left side of a neurostimulation device can comprise: a first arm/portion which curves forward and downward from a person's ear and holds at least one first electromagnetic energy emitter (substantially located at an electrode position selected from the group consisting of T9 and T7); a second arm/portion which curves backward and downward from the person's ear; and a third arm/portion which loops around the rear of the person's head.

In an example, the left side of a neurostimulation device can comprise: a first arm/portion which curves forward and downward from a person's ear; a second arm/portion which curves backward and downward from the person's ear and holds at least one second electromagnetic energy emitter (substantially located at an electrode position selected from the group consisting of Iz, P7, TP7); and a third arm/portion which loops around the rear of the person's head.

In an example, the left side of a neurostimulation device can comprise: a first arm/portion which curves forward and downward from a person's ear; a second arm/portion which curves backward and downward from the person's ear; and a third arm/portion which loops around the rear of the person's head and holds at least one third electromagnetic energy emitter (substantially located at an electrode position selected from the group consisting of TP9 and TP7).

In an example, the first arm/portion curves forward and downward in a sinusoidal manner, comprising a partial or complete sinusoidal wave. In an example, the first arm/portion curves forward and downward from the top of the person's ear. In an example, the first arm/portion has a length between 2″ and 5.″ In an example, the second arm/portion curves backward and downward from the top of the person's ear. In an example, the second arm/portion curves around the rear of the person's ear. In an example, the third arm/portion tilts downward as is spans from the person's ear to the rear of the person's head. In an example, the first, second, and third arms/portions are connected and/or joined at a location (directly) above the person's ear. In an example, the neurostimulation device can rest on the tops of the person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 15 shows the left side of a neurostimulation device comprising: a forehead arm/portion 1501 which spans from a person's ear to their forehead and holds at least one first electromagnetic energy emitter 1511 (substantially located at an electrode position selected from the group consisting of FP1, FPz, AF7, F7, FT7, and T7); a front-ear arm/portion 1502 which curves around the front of the person's ear and holds at least one second electromagnetic energy emitter 1512 (substantially located at an electrode position selected from the group consisting of T9 and T7); and a rear-ear arm/portion 1503 which curves around the rear of the person's ear and holds at least one third electromagnetic energy emitter 1513 (substantially located at an electrode position selected from the group consisting of TP9, TP7, and T7).

In an example, the left side of a neurostimulation device can comprise: a forehead arm/portion which spans from a person's ear to their forehead and holds at least one first electromagnetic energy emitter (substantially located at an electrode position selected from the group consisting of FP1, FPz, AF7, F7, FT7, and T7); a front-ear arm/portion which curves around the front of the person's ear; and a rear-ear arm/portion which curves around the rear of the person's ear.

In an example, the left side of a neurostimulation device can comprise: a forehead arm/portion which spans from a person's ear to their forehead; a front-ear arm/portion which curves around the front of the person's ear and holds at least one second electromagnetic energy emitter (substantially located at an electrode position selected from the group consisting of T9 and T7); and a rear-ear arm/portion which curves around the rear of the person's ear.

In an example, the left side of a neurostimulation device can comprise: a forehead arm/portion which spans from a person's ear to their forehead; a front-ear arm/portion which curves around the front of the person's ear; and a rear-ear arm/portion which curves around the rear of the person's ear and holds at least one third electromagnetic energy emitter (substantially located at an electrode position selected from the group consisting of TP9, TP7, and T7).

In an example, the forehead arm/portion can loop laterally (e.g. left to right) around the person's forehead. In an example, the three arms/portions of this neurostimulation device can connect and/or intersect at a location (directly) above the person's ear. In an example, front-ear and rear-ear arms/portions can combine to span between 50% and 75% of the circumference of the person's ear. In an example, front-ear and rear-ear can combine to span between 66% and 95% of the circumference of the person's ear. In an example, the forehead arm/portion can have an upward-facing concavity. In an example, the neurostimulation device can rest on the tops of the person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 16 shows the left side of a neurostimulation device comprising: an upper loop 1601 which loops over a person' head from a location within 2″ of a person's ear and holds at least one first electromagnetic energy emitter 1611 (substantially located at an electrode position selected from the group consisting of TP7, CP5, CP3, CP1, and CPz); a front-ear arm/portion 1602 which curves around the front of the person's ear and holds at least one second electromagnetic energy emitter 1612 (substantially located at an electrode position selected from the group consisting of T9 and T7); and a rear-ear arm/portion 1603 which curves around the rear of the person's ear and holds at least one third electromagnetic energy emitter 1613 (substantially located at an electrode position selected from the group consisting of TP9 and TP7).

In an example, the left side of a neurostimulation device can comprise: an upper loop which loops over a person' head from a location within 2″ of a person's ear and holds at least one first electromagnetic energy emitter (substantially located at an electrode position selected from the group consisting of TP7, CP5, CP3, CP1, and CPz); a front-ear arm/portion which curves around the front of the person's ear; and a rear-ear arm/portion which curves around the rear of the person's ear.

In an example, the left side of a neurostimulation device can comprise: an upper loop which loops over a person' head from a location within 2″ of a person's ear; a front-ear arm/portion which curves around the front of the person's ear and holds at least one second electromagnetic energy emitter (substantially located at an electrode position selected from the group consisting of T9 and T7); and a rear-ear arm/portion which curves around the rear of the person's ear.

In an example, the left side of a neurostimulation device can comprise: an upper loop which loops over a person' head from a location within 2″ of a person's ear; a front-ear arm/portion which curves around the front of the person's ear; and a rear-ear arm/portion which curves around the rear of the person's ear and holds at least one third electromagnetic energy emitter (substantially located at an electrode position selected from the group consisting of TP9 and TP7).

In an example, the upper loop can loop over a person's head from a location (directly) above a person's ear. In an example, the upper loop can have a forward-facing concavity. In an example, the upper loop can have a sinusoidal shape. In an example, the upper loop can span the top of the person's head at a location to the rear of a virtual vertical plane which connects the person's ears. In an example, front-ear and rear-ear arms/portions can combine to span between 50% and 75% of the circumference of the person's ear. In an example, front-ear and rear-ear arms/portions can combine to span between 66% and 95% of the circumference of the person's ear. In an example, the neurostimulation device can rest on the tops of the person's ears. In an example, the neurostimulation device can comprise (or just be shaped like) a set of headphones.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 17 shows the left side of a neurostimulation device comprising: a forward arm/portion 1701 which loops around an upper-frontal area of a person's head and holds at least one first electromagnetic energy emitter 1711 (substantially located at an electrode position selected from the group consisting of F7, F5, F3, F1, and Fz); a middle arm/portion 1702 which laterally spans a portion of the side of the person's head above their ear and holds at least one second electromagnetic energy emitter 1712 (substantially located at an electrode position selected from the group consisting of F7, TP7, FT7, and T7); and a rear arm/portion 1703 which loops around an upper-rear area of the person's head and holds at least one third electromagnetic energy emitter 1713 (substantially located at an electrode position selected from the group consisting of TP7, CP5, CP3, CP1, and CPz), wherein the middle arm/portion connects the forward arm/portion and the rear arm/portion.

In an example, the left side of a neurostimulation device can comprise: a forward arm/portion which loops around an upper-frontal area of a person's head and holds at least one first electromagnetic energy emitter (substantially located at an electrode position selected from the group consisting of F7, F5, F3, F1, and Fz); a middle arm/portion which laterally spans a portion of the side of the person's head above their ear; and a rear arm/portion which loops around an upper-rear area of the person's head, wherein the middle arm/portion connects the forward arm/portion and the rear arm/portion.

In an example, the left side of a neurostimulation device can comprise: a forward arm/portion which loops around an upper-frontal area of a person's head; a middle arm/portion which laterally spans a portion of the side of the person's head above their ear and holds at least one second electromagnetic energy emitter (substantially located at an electrode position selected from the group consisting of F7, TP7, FT7, and T7); and a rear arm/portion which loops around an upper-rear area of the person's head, wherein the middle arm/portion connects the forward arm/portion and the rear arm/portion.

In an example, the left side of a neurostimulation device can comprise: a forward arm/portion which loops around an upper-frontal area of a person's head; a middle arm/portion which laterally spans a portion of the side of the person's head above their ear; and a rear arm/portion which loops around an upper-rear area of the person's head and holds at least one third electromagnetic energy emitter (substantially located at an electrode position selected from the group consisting of TP7, CP5, CP3, CP1, and CPz), wherein the middle arm/portion connects the forward arm/portion and the rear arm/portion.

In an example, the centroid of the middle arm/portion is below the centroid of the forward arm/portion and the centroid of the rear arm/portion. In an example, the middle arm/portion can rest on top of the person's ear. In an example, the neurostimulation device can be shaped like a saddle on top of the person's head. In an example, the forward arm/portion can span the top of the person's head at a location which is anterior to a virtual vertical plane which connects the person's ears. In an example, the rear arm/portion can span the top of the person's head at a location which is posterior to a virtual vertical plane which connects the person's ears. In an example, the forward arm/portion can have a forward-facing concavity. In an example, the rear arm/portion can have a rear-facing concavity. In an example, the middle arm/portion, the forward arm/portion, and the rear arm/portion can each have substantially the same length.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 18 shows the left side of a neurostimulation device comprising: a forward arm/portion 1801 which loops around an upper-frontal area of a person's head and holds at least one first electromagnetic energy emitter 1811 (substantially located at an electrode position selected from the group consisting of FT7, FC5, FC3, FC1, and FCz); a middle arm/portion 1802 which laterally spans a portion of the side of the person's head above their ear and holds at least one second electromagnetic energy emitter 1812 (substantially located at an electrode position selected from the group consisting of TP7, FT7, and T7); and a rear arm/portion 1803 which loops around an upper-rear area of the person's head and holds at least one third electromagnetic energy emitter 1813 (substantially located at an electrode position selected from the group consisting of TP7, CP5, CP3, CP1, and CPz), wherein the middle arm/portion connects the forward arm/portion and the rear arm/portion.

In an example, the left side of a neurostimulation device can comprise: a forward arm/portion which loops around an upper-frontal area of a person's head and holds at least one first electromagnetic energy emitter (substantially located at an electrode position selected from the group consisting of FT7, FC5, FC3, FC1, and FCz); a middle arm/portion which laterally spans a portion of the side of the person's head above their ear; and a rear arm/portion which loops around an upper-rear area of the person's head, wherein the middle arm/portion connects the forward arm/portion and the rear arm/portion.

In an example, the left side of a neurostimulation device can comprise: a forward arm/portion which loops around an upper-frontal area of a person's head; a middle arm/portion which laterally spans a portion of the side of the person's head above their ear and holds at least one second electromagnetic energy emitter (substantially located at an electrode position selected from the group consisting of TP7, FT7, and T7); and a rear arm/portion which loops around an upper-rear area of the person's head, wherein the middle arm/portion connects the forward arm/portion and the rear arm/portion.

In an example, the left side of a neurostimulation device can comprise: a forward arm/portion which loops around an upper-frontal area of a person's head; a middle arm/portion which laterally spans a portion of the side of the person's head above their ear; and a rear arm/portion which loops around an upper-rear area of the person's head and holds at least one third electromagnetic energy emitter (substantially located at an electrode position selected from the group consisting of TP7, CP5, CP3, CP1, and CPz), wherein the middle arm/portion connects the forward arm/portion and the rear arm/portion.

In an example, the centroid of the middle arm/portion is below the centroid of the forward arm/portion and the centroid of the rear arm/portion. In an example, the middle arm/portion can rest on top of the person's ear. In an example, the neurostimulation device can be shaped like a saddle on top of the person's head. In an example, the forward arm/portion can span the top of the person's head at a location which is anterior to a virtual vertical plane which connects the person's ears. In an example, the rear arm/portion can span the top of the person's head at a location which is posterior to a virtual vertical plane which connects the person's ears. In an example, the forward arm/portion can have a forward-facing concavity. In an example, the rear arm/portion can have a rear-facing concavity. In an example, the middle arm/portion can be shorter than either the forward arm/portion or the rear arm/portion.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

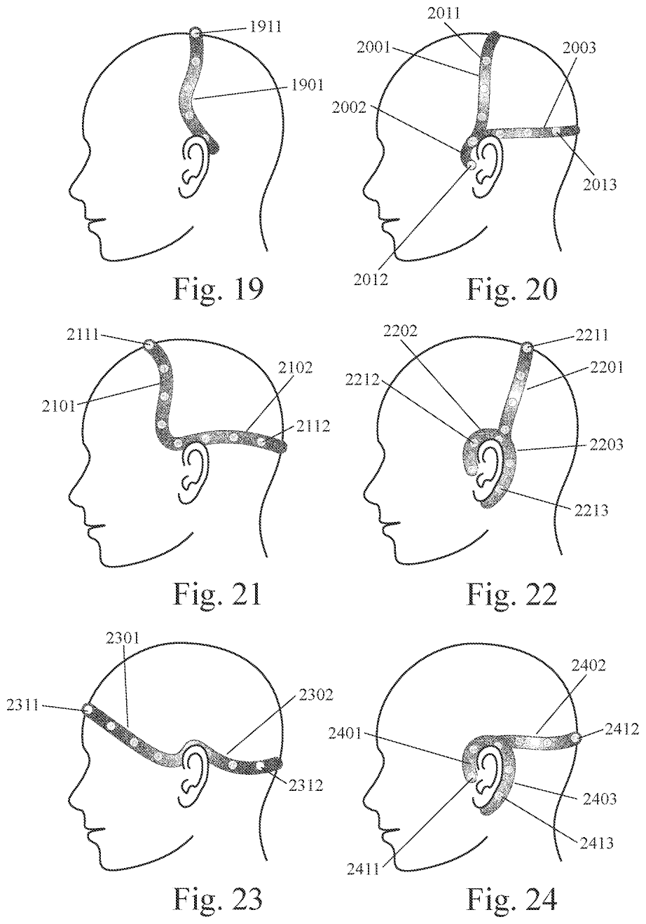

FIG. 19 shows the left side of a neurostimulation device comprising: an upper loop 1901 which loops over the top of a person's head from one side (e.g. left) side to the other side (e.g. right) and wherein holds at least one first electromagnetic energy emitter 1911 (substantially located at an electrode position selected from the group consisting of TP9, TP7, T7, C5, C3, C1, and Cz).

In an example, the upper loop can loop over the top of the person's head from one ear to the other ear. In an example, the upper loop can loop over the top of the person's head from a location (directly) above an ear. In an example, this loop can have a rear-facing concavity. In an example, this loop can have a forward-facing concavity. In an example, this loop can have a sinusoidal shape. In an example, the virtual plane which best fits the upper loop can be substantially vertical. In an example, the virtual plane which best fits the upper loop can be a virtual vertical plane which connects the person's ears. In an example, the upper loop can cross the top of the person's head in a virtual vertical plane which connects the person's ears. In an example, the virtual plane which best fits the upper loop can intersect a vertical plane at an acute angle (e.g. between 5 and 45 degrees).

In an example, the right side of this neurostimulation device (not shown) can be laterally symmetric with respect to the left side. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 20 shows the left side of a neurostimulation device comprising: an upper loop 2001 which loops over the top of a person's head (from a location over the person's ear) and holds at least one first electromagnetic energy emitter 2011 (substantially located at an electrode position selected from the group consisting of T7, C5, C3, C1, and Cz); a rear loop 2002 which loops around the rear of the person's head (from the location over the person's ear) and holds at least one second electromagnetic energy emitter 2012 (substantially located at an electrode position selected from the group consisting of T9 and T7); an front-ear arm/portion 2003 which curves around the front of the person's ear and holds at least one third electromagnetic energy emitter 2013 (substantially located at an electrode position selected from the group consisting of O1, Oz, PO7, P7, TP7, and T7).

In an example, the left side of a neurostimulation device can comprise: an upper loop which loops over the top of a person's head (from a location over the person's ear) and holds at least one first electromagnetic energy emitter (substantially located at an electrode position selected from the group consisting of T7, C5, C3, C1, and Cz); a rear loop which loops around the rear of the person's head (from the location over the person's ear); an front-ear arm/portion which curves around the front of the person's ear.

In an example, the left side of a neurostimulation device can comprise: an upper loop which loops over the top of a person's head (from a location over the person's ear); a rear loop which loops around the rear of the person's head (from the location over the person's ear) and holds at least one second electromagnetic energy emitter (substantially located at an electrode position selected from the group consisting of T9 and T7); an front-ear arm/portion which curves around the front of the person's ear.

In an example, the left side of a neurostimulation device can comprise: an upper loop which loops over the top of a person's head (from a location over the person's ear); a rear loop which loops around the rear of the person's head (from the location over the person's ear); an front-ear arm/portion which curves around the front of the person's ear and holds at least one third electromagnetic energy emitter (substantially located at an electrode position selected from the group consisting of O1, Oz, PO7, P7, TP7, and T7).

In an example, the upper loop can loop over the top of the person's head from one ear to the other ear. In an example, the upper loop can loop over the top of the person's head from a location (directly) above an ear. In an example, this loop can have a rear-facing concavity. In an example, this loop can have a forward-facing concavity. In an example, this loop can have a sinusoidal shape. In an example, the virtual plane which best fits the upper loop can be substantially vertical. In an example, the virtual plane which best fits the upper loop can be a virtual vertical plane which connects the person's ears. In an example, the upper loop can cross the top of the person's head in a virtual vertical plane which connects the person's ears. In an example, the virtual plane which best fits the upper loop can intersect a vertical plane at an acute angle (e.g. between 5 and 45 degrees). In an example, the upper loop, rear loop, and front-ear arm/portion can intersect and/or connect at a location within 2″ of the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 21 shows the left side of a neurostimulation device comprising: a first arm/portion 2101 which spans from a location within 2″ of a person's ear to the top of the person's head and holds at least one first electromagnetic energy emitter 2111 (substantially located at an electrode position selected from the group consisting of FT7, FC5, FC3, FC1, FCz, and T7); and a second arm/portion 2102 which spans from the location to the rear of the person's head and holds at least one second electromagnetic energy emitter 2112 (substantially located at an electrode position selected from the group consisting of Iz, O1, Oz, PO7, P7, TP7, and T7).

In an example, the left side of a neurostimulation device can comprise: a first arm/portion which spans from a location within 2″ of a person's ear to the top of the person's head and holds at least one first electromagnetic energy emitter (substantially located at an electrode position selected from the group consisting of FT7, FC5, FC3, FC1, FCz, and T7); and a second arm/portion which spans from the location to the rear of the person's head.

In an example, the left side of a neurostimulation device can comprise: a first arm/portion which spans from a location within 2″ of a person's ear to the top of the person's head; and a second arm/portion which spans from the location to the rear of the person's head and holds at least one second electromagnetic energy emitter (substantially located at an electrode position selected from the group consisting of Iz, O1, Oz, PO7, P7, TP7, and T7).

In an example, the first arm/portion can loop over the top of the person's head. In an example, the first arm/portion can have a forward-facing concavity. In an example, the second arm/portion can loop around the rear of the person's head. In an example, the second arm/portion can have a downward-facing concavity. In an example, the neurostimulation device overall (including all portions) can have an upward-and-rear-facing concavity. In an example, the best fitting virtual plane for the first arm/portion and the best fitting virtual plane for the second arm/portion can be substantially perpendicular to each other. In an example, this neurostimulation device can rest on the tops of the person's ears. In an example, an overall neurostimulation device (including both left and right sides) can comprise an undulating (e.g. sinusoidal) ring around an upper rear portion of a person's head.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 22 shows the left side of a neurostimulation device comprising: a first arm/portion 2201 which spans from an area above a person's ear to the top of their head and holds at least one first electromagnetic energy emitter 2211 (substantially located at an electrode position selected from the group consisting of TP7, CP5, CP3, CP1, and CPz); a second arm/portion 2202 which curves around the front of the person's ear and holds at least one second electromagnetic energy emitter 2212 (substantially located at an electrode position selected from the group consisting of TP7, T9, and T7); and a third arm/portion 2203 which curves around the rear of the person's ear and holds at least one third electromagnetic energy emitter 2213 (substantially located at an electrode position selected from the group consisting of P9, TP9, and TP7).

In an example, the left side of a neurostimulation device can comprise: a first arm/portion which spans from an area above a person's ear to the top of their head and holds at least one first electromagnetic energy emitter (substantially located at an electrode position selected from the group consisting of TP7, CP5, CP3, CP1, and CPz); a second arm/portion which curves around the front of the person's ear; and a third arm/portion which curves around the rear of the person's ear.

In an example, the left side of a neurostimulation device can comprise: a first arm/portion which spans from an area above a person's ear to the top of their head; a second arm/portion which curves around the front of the person's ear and holds at least one second electromagnetic energy emitter (substantially located at an electrode position selected from the group consisting of TP7, T9, and T7); and a third arm/portion which curves around the rear of the person's ear.

In an example, the left side of a neurostimulation device can comprise: a first arm/portion which spans from an area above a person's ear to the top of their head; a second arm/portion which curves around the front of the person's ear; and a third arm/portion which curves around the rear of the person's ear and holds at least one third electromagnetic energy emitter (substantially located at an electrode position selected from the group consisting of P9, TP9, and TP7).

In an example, this neurostimulation device can comprise a pair of headphones. In an example, the first arm/portion can loop over the top of the person's head. In an example, the first arm/portion can have a rearward tilt as it ascends the side of the person's head. In an alternative example, the first arm/portion can be substantially vertical. In an example, the first arm/portion can have a forward-facing concavity. In an example, the first arm/portion can have a rear-facing concavity. In an example, the second and third arms/portions can collectively span between one half and three-quarters of the circumference around a person's ear. In an example, the first, second, and third arms/portions of this neurostimulation device can intersect and/or be connected to each other at an area (directly) above the person's ear. In an example, the third arm/portion can also hold an electrode which is attached to the person's ear lobe.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 23 shows the left side of a neurostimulation device comprising: a front portion/loop 2301, which spans from a person's ear to their forehead and holds at least one first electromagnetic energy emitter 2311 (substantially located at an electrode position selected from the group consisting of FP1, FPz, AF7, F9, F7, FT9, T9, and T7); and a rear portion/loop 2302 which spans from the person's ear to the rear of the person's head and holds at least one third electromagnetic energy emitter 2312 (substantially located at an electrode position selected from the group consisting of Iz, P9, TP9, and T7).

In an example, the left side of a neurostimulation device can comprise: a front portion/loop which spans from a person's ear to their forehead and holds at least one first electromagnetic energy emitter (substantially located at an electrode position selected from the group consisting of FP1, FPz, AF7, F9, F7, FT9, T9, and T7); and a rear portion/loop which spans from the person's ear to the rear of the person's head.

In an example, the left side of a neurostimulation device can comprise: a front portion/loop which spans from a person's ear to their forehead; and a rear portion/loop which spans from the person's ear to the rear of the person's head and holds at least one third electromagnetic energy emitter (substantially located at an electrode position selected from the group consisting of Iz, P9, TP9, and T7).

In an example, this neurostimulation device can comprise an undulating ring which encircles a person's head, spanning their forehead and the rear of their head, with upward undulations over the tops of the person's ears. In an example, this neurostimulation device can comprise an undulating ring which encircles a person's head, spanning their forehead and the rear of their head, with sinusoidal waves over the tops of the person's ears. In an example, this neurostimulation device can comprise a ring or headband around a person's head with downward-facing concave portions over the person's ears. In an example, the front portion/loop can loop around the person's forehead and the rear portion/loop can loop around the rear of the person's head. In an example, a virtual plane which best fits this undulating ring can intersect a horizontal plane when the person is standing up, forming a forward-facing angle between 5 and 75 degrees. In an example, the front of this undulating ring can be higher than the rear of this undulating ring. In an example, the rear of the undulating ring can be lower than the top of the person's ear. In an example, this neurostimulation device can comprise a headband or a cap.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 24 shows the left side of a neurostimulation device comprising: a first arm/portion 2401 which curves around the front of a person's ear and holds at least one first electromagnetic energy emitter 2411 (substantially located at an electrode position selected from the group consisting of T9 and T7); a second arm/portion 2402 which curves around the rear of the person's ear and holds at least one second electromagnetic energy emitter 2412 (substantially located at an electrode position selected from the group consisting of O1, Oz, PO7, P7, TP7, and T7); a third arm/portion 2403 which extends from the person's ear to the rear of their head and holds at least one third electromagnetic energy emitter 2413 (substantially located at an electrode position selected from the group consisting of P9, TP9, and TP7).

In an example, the left side of a neurostimulation device can comprise: a first arm/portion which curves around the front of a person's ear and holds at least one first electromagnetic energy emitter (substantially located at an electrode position selected from the group consisting of T9 and T7); a second arm/portion which curves around the rear of the person's ear; a third arm/portion which extends from the person's ear to the rear of their head.

In an example, the left side of a neurostimulation device can comprise: a first arm/portion which curves around the front of a person's ear; a second arm/portion which curves around the rear of the person's ear and holds at least one second electromagnetic energy emitter (substantially located at an electrode position selected from the group consisting of O1, Oz, PO7, P7, TP7, and T7); a third arm/portion which extends from the person's ear to the rear of their head.

In an example, the left side of a neurostimulation device can comprise: a first arm/portion which curves around the front of a person's ear; a second arm/portion which curves around the rear of the person's ear; a third arm/portion which extends from the person's ear to the rear of their head and holds at least one third electromagnetic energy emitter (substantially located at an electrode position selected from the group consisting of P9, TP9, and TP7).

In an example, the first arm/portion curves forward and downward in a sinusoidal manner around the front of the person's ear, comprising a partial or complete sinusoidal wave. In an example, the first arm/portion has a length between 1″ and 3.″ In an example, the second arm/portion curves backward and downward from the top of the person's ear around the rear of the person's ear. In an example, the third arm/portion is relatively horizontal as loops around the rear of the person's head from ear to ear. In an example, the first, second, and third arms/portions are connected and/or joined at a location (directly) above the person's ear. In an example, this neurostimulation device can rest on the person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

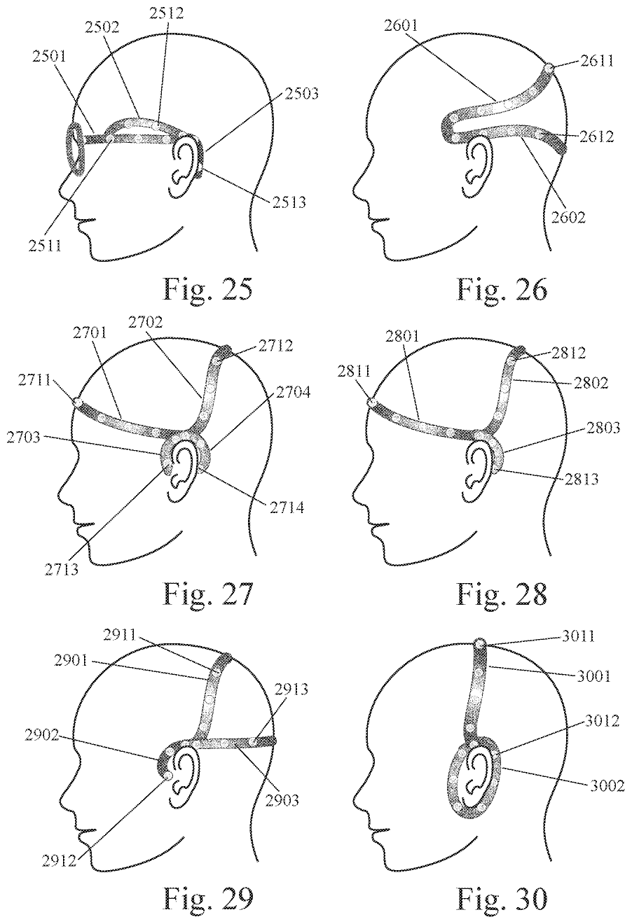

FIG. 25 shows the left side of a neurostimulation device comprising: a lower branch of an eyewear sidepiece 2501 which holds at least one first electromagnetic energy emitter 2511 (substantially located at an electrode position selected from the group consisting of Nz, F9, FT9, FT7, and T7); an upper branch of the eyewear sidepiece 2502 which holds at least one second electromagnetic energy emitter 2512 (substantially located at an electrode position selected from the group consisting of AF7, F7, FT7, and T7); and a rear-ear arm of the eyewear sidepiece 2503 which holds at least one third electromagnetic energy emitter 2513 (substantially located at an electrode position selected from the group consisting of P9, TP9, and TP7).

In an example, the left side of a neurostimulation device can comprise: a lower branch of an eyewear sidepiece which holds at least one first electromagnetic energy emitter (substantially located at an electrode position selected from the group consisting of Nz, F9, FT9, FT7, and T7); an upper branch of the eyewear sidepiece; and a rear-ear arm of the eyewear sidepiece.

In an example, the left side of a neurostimulation device can comprise: a lower branch of an eyewear sidepiece; an upper branch of the eyewear sidepiece which holds at least one second electromagnetic energy emitter (substantially located at an electrode position selected from the group consisting of AF7, F7, FT7, and T7); and a rear-ear arm of the eyewear sidepiece.

In an example, the left side of a neurostimulation device can comprise: a lower branch of an eyewear sidepiece; an upper branch of the eyewear sidepiece; and a rear-ear arm of the eyewear sidepiece which holds at least one third electromagnetic energy emitter (substantially located at an electrode position selected from the group consisting of P9, TP9, and TP7).

In an example, the upper branch of the sidepiece has a front end and a rear end which are both connected to the lower branch of the sidepiece. In an example, the lower branch of the sidepiece is relatively straight and the upper branch of the sidepiece is arcuate. In an example, the upper branch of the sidepiece has a sinusoidal shape. In an example, the upper branch of the sidepiece has a conic section shape. In an example, the sidepiece can be called the “temple” of a pair of eyeglasses. In an example, the upper branch is shorter than the lower branch.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 26 shows the left side of a neurostimulation device comprising: an upper arm/portion 2601 which spans from a location between a person's temple and the person's ear to the back of their head and holds at least one first electromagnetic energy emitter 2611 (substantially located at an electrode position selected from the group consisting of P5, P3, P1, Pz, CP5, FT7, FC5, and C5); and a lower arm/portion 2602 which also spans from the location to the back of their head and holds at least one second electromagnetic energy emitter 2612 (substantially located at an electrode position selected from the group consisting of Iz, O1, PO7, P7, TP7, FT7, and T7), wherein the front of the upper arm/portion is connected to the front of the lower arm/portion, and wherein the rear of the upper arm/portion is higher than the rear of the lower arm/portion.

In an example, the left side of a neurostimulation device can comprise: an upper arm/portion which spans from a location between a person's temple and the person's ear to the back of their head and holds at least one first electromagnetic energy emitter (substantially located at an electrode position selected from the group consisting of P5, P3, P1, Pz, CP5, FT7, FC5, and C5); and a lower arm/portion which also spans from the location to the back of their head, wherein the front of the upper arm/portion is connected to the front of the lower arm/portion, and wherein the rear of the upper arm/portion is higher than the rear of the lower arm/portion.

In an example, the left side of a neurostimulation device can comprise: an upper arm/portion which spans from a location between a person's temple and the person's ear to the back of their head and holds at least one first electromagnetic energy emitter; and a lower arm/portion which also spans from the location to the back of their head and holds at least one second electromagnetic energy emitter (substantially located at an electrode position selected from the group consisting of Iz, O1, PO7, P7, TP7, FT7, and T7), wherein the front of the upper arm/portion is connected to the front of the lower arm/portion, and wherein the rear of the upper arm/portion is higher than the rear of the lower arm/portion.

In an example, the upper arm/portion can have an upward-facing concavity and the lower arm/portion can have a downward-facing concavity. In an example, the neurostimulation device can span between one-half and two-thirds of the front-to-back width of the side of the person's head. In an example, the lower arms/portions of the neurostimulation device can rest on the person's ears. In an example, the neurostimulation device overall can have a rear-facing concavity. In an example, the left-side-portion of the neurostimulation device can have the shape of a rear-opening “bobby pin.”

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 27 shows the left side of a neurostimulation device comprising: a first arm/portion 2701 which extends from a person's ear to their forehead (e.g. loops around their forehead) and holds at least one first electromagnetic energy emitter 2711 (substantially located at an electrode position selected from the group consisting of FP1, FPz, AF7, F7, TP7, FT7, and T7); a second arm/portion 2702 which extends from the person's ear to the top of their head (e.g. loops around the top of their head) and holds at least one second electromagnetic energy emitter 2712 (substantially located at an electrode position selected from the group consisting of TP7, CP5, CP3, CP1, and CPz); a third arm/portion 2703 which curves around the front of the person's ear and holds at least one third electromagnetic energy emitter 2713 (substantially located at an electrode position selected from the group consisting of T9 and T7); and a fourth arm/portion 2704 which curves around the rear of the person's ear and holds at least one fourth electromagnetic energy emitter 2714 (substantially located at an electrode position selected from the group consisting of P9, TP9, and TP7).

In an example, the left side of a neurostimulation device can comprise: a first arm/portion which extends from a person's ear to their forehead (e.g. loops around their forehead) and holds at least one first electromagnetic energy emitter (substantially located at an electrode position selected from the group consisting of FP1, FPz, AF7, F7, TP7, FT7, and T7); a second arm/portion which extends from the person's ear to the top of their head (e.g. loops around the top of their head); a third arm/portion which curves around the front of the person's ear; and a fourth arm/portion which curves around the rear of the person's ear.

In an example, the left side of a neurostimulation device can comprise: a first arm/portion which extends from a person's ear to their forehead (e.g. loops around their forehead); a second arm/portion which extends from the person's ear to the top of their head (e.g. loops around the top of their head) and holds at least one second electromagnetic energy emitter (substantially located at an electrode position selected from the group consisting of TP7, CP5, CP3, CP1, and CPz); a third arm/portion which curves around the front of the person's ear; and a fourth arm/portion which curves around the rear of the person's ear.

In an example, the left side of a neurostimulation device can comprise: a first arm/portion which extends from a person's ear to their forehead (e.g. loops around their forehead); a second arm/portion which extends from the person's ear to the top of their head (e.g. loops around the top of their head); a third arm/portion which curves around the front of the person's ear and holds at least one third electromagnetic energy emitter (substantially located at an electrode position selected from the group consisting of T9 and T7); and a fourth arm/portion which curves around the rear of the person's ear.

In an example, the left side of a neurostimulation device can comprise: a first arm/portion which extends from a person's ear to their forehead (e.g. loops around their forehead); a second arm/portion which extends from the person's ear to the top of their head (e.g. loops around the top of their head); a third arm/portion which curves around the front of the person's ear; and a fourth arm/portion which curves around the rear of the person's ear and holds at least one fourth electromagnetic energy emitter (substantially located at an electrode position selected from the group consisting of P9, TP9, and TP7).

In an example, the first arm/portion can loop around the person's forehead. In an example, the second arm/portion can loop around the top of the person's head. In an example, the best fitting virtual plane for the first arm/portion and the best fitting virtual plane for the second arm/portion can be substantially perpendicular to each other. In an example, the first arm/portion can have an upward-facing concavity. In an example, the second arm/portion can have a rear-facing concavity. In an example, the second arm/portion can have a forward-facing concavity.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 28 shows the left side of a neurostimulation device comprising: a first arm/portion 2801 which extends from a person's ear to their forehead (e.g. loops around their forehead) and holds at least one first electromagnetic energy emitter 2811 (substantially located at an electrode position selected from the group consisting of FP1, FPz, AF7, F7, TP7, FT7, and T7); a second arm/portion 2802 which extends from the person's ear to the top of their head (e.g. loops around the top of their head) and holds at least one second electromagnetic energy emitter 2812 (substantially located at an electrode position selected from the group consisting of TP7, CP5, CP3, CP1, CPz); and a third arm/portion 2803 which curves around the rear of the person's ear and holds at least one third electromagnetic energy emitter 2813 (substantially located at an electrode position selected from the group consisting of TP9, TP7, and T7).

In an example, the left side of a neurostimulation device can comprise: a first arm/portion which extends from a person's ear to their forehead (e.g. loops around their forehead) and holds at least one first electromagnetic energy emitter (substantially located at an electrode position selected from the group consisting of FP1, FPz, AF7, F7, TP7, FT7, and T7); a second arm/portion which extends from the person's ear to the top of their head (e.g. loops around the top of their head); and a third arm/portion which curves around the rear of the person's ear.

In an example, the left side of a neurostimulation device can comprise: a first arm/portion which extends from a person's ear to their forehead (e.g. loops around their forehead); a second arm/portion which extends from the person's ear to the top of their head (e.g. loops around the top of their head) and holds at least one second electromagnetic energy emitter (substantially located at an electrode position selected from the group consisting of TP7, CP5, CP3, CP1, CPz); and a third arm/portion which curves around the rear of the person's ear.

In an example, the left side of a neurostimulation device can comprise: a first arm/portion which extends from a person's ear to their forehead (e.g. loops around their forehead); a second arm/portion which extends from the person's ear to the top of their head (e.g. loops around the top of their head); and a third arm/portion which curves around the rear of the person's ear and holds at least one third electromagnetic energy emitter (substantially located at an electrode position selected from the group consisting of TP9, TP7, and T7).

In an example, the first arm/portion can loop around the person's forehead. In an example, the second arm/portion can loop around the top of the person's head. In an example, the best fitting virtual plane for the first arm/portion and the best fitting virtual plane for the second arm/portion can be substantially perpendicular to each other. In an example, the first arm/portion can have an upward-facing concavity. In an example, the second arm/portion can have a rear-facing concavity. In an example, the second arm/portion can have a forward-facing concavity.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 29 shows the left side of a neurostimulation device comprising: a first arm/portion 2901 which loops over the top of a person's head from their ear and holds at least one first electromagnetic energy emitter 2911 (substantially located at an electrode position selected from the group consisting of TP7, CP5, CP3, CP1, and CPz); a second arm/portion 2902 which loops around the back of the person's head from their ear and holds at least one second electromagnetic energy emitter 2912 (substantially located at an electrode position selected from the group consisting of T9, T7); and a third arm/portion 2903 which curves around the front of the person's ear and holds at least one third electromagnetic energy emitter 2913 (substantially located at an electrode position selected from the group consisting of Iz, O1, Oz, PO7, P7, TP7, and T7).

In an example, the left side of a neurostimulation device can comprise: a first arm/portion which loops over the top of a person's head from their ear and holds at least one first electromagnetic energy emitter (substantially located at an electrode position selected from the group consisting of TP7, CP5, CP3, CP1, and CPz); a second arm/portion which loops around the back of the person's head from their ear; and a third arm/portion which curves around the front of the person's ear.

In an example, the left side of a neurostimulation device can comprise: a first arm/portion which loops over the top of a person's head from their ear; a second arm/portion which loops around the back of the person's head from their ear and holds at least one second electromagnetic energy emitter (substantially located at an electrode position selected from the group consisting of T9, T7); and a third arm/portion which curves around the front of the person's ear.

In an example, the left side of a neurostimulation device can comprise: a first arm/portion which loops over the top of a person's head from their ear; a second arm/portion which loops around the back of the person's head from their ear; and a third arm/portion which curves around the front of the person's ear and holds at least one third electromagnetic energy emitter (substantially located at an electrode position selected from the group consisting of Iz, O1, Oz, PO7, P7, TP7, and T7).

In an example, the first arm/portion can have a rear-facing concavity. In an example, the first arm/portion can have an undulating shape. In an example, the first arm/portion can have a sinusoidal shape. In an example, the second arm/portion can be substantially level/horizontal. In an example, the virtual plane which best fits the first arm/portion can intersect the virtual plane which best fits the second arm/portion at a rear-facing angle between 45 and 90 degrees. In an example, the first arm/portion can loop over the top of the person's head. In an example, the first arm/portion can have posterior tilt as it extends upward to loop over the top of the person's head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 30 shows the left side of a neurostimulation device comprising: an upper loop 3001 which loops over a person's head from one ear to the other and holds at least one first electromagnetic energy emitter 3011 (substantially located at an electrode position selected from the group consisting of T7, C5, C3, C1, and Cz); and an ear loop 3002 which encircles the person' ear and holds at least one second electromagnetic energy emitter 3012 (substantially located at an electrode position selected from the group consisting of TP9, TP7, T9, and T7).

In an example, a neurostimulation device can comprise: an upper loop which loops over a person's head from one ear to the other and holds at least one first electromagnetic energy emitter (substantially located at an electrode position selected from the group consisting of T7, C5, C3, C1, and Cz); and an ear loop which encircles the person' ear.

In an example, a neurostimulation device can comprise: an upper loop which loops over a person's head from one ear to the other; and an ear loop which encircles the person' ear and holds at least one second electromagnetic energy emitter (substantially located at an electrode position selected from the group consisting of TP9, TP7, T9, and T7).

In an example the upper loop can be substantially vertical. In an example, the upper loop can have a forward-facing concavity. In an example, the upper loop can have a rear-facing concavity. In an example, the neurostimulation device can comprise (or be shaped like) a pair of headphones. In an example, the ear loop can hold an electrode in contact with the person's earlobe.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

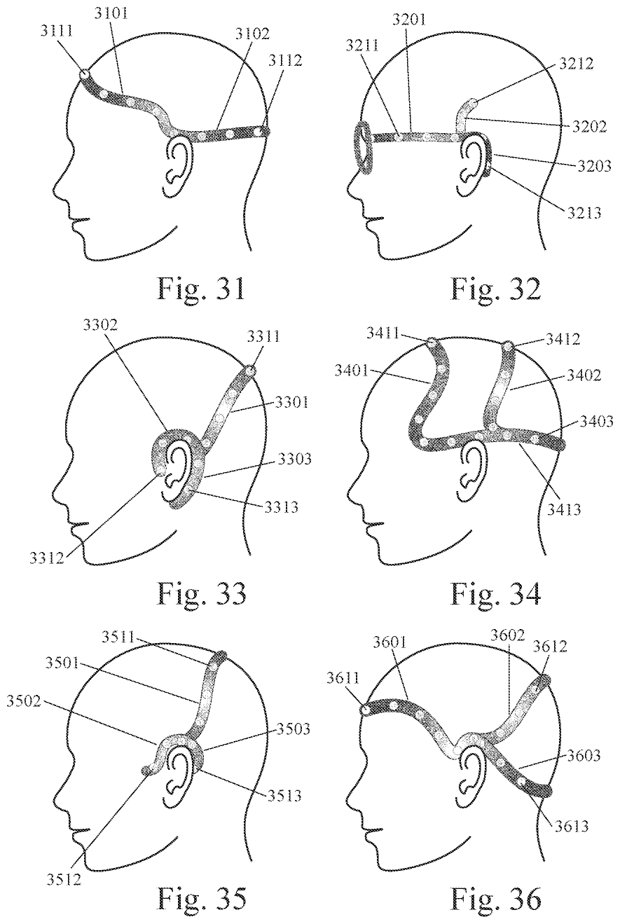

FIG. 31 shows the left side of a neurostimulation device comprising: a forward loop/portion 3101 which loops around the front of a person's head from a location within 2″ of their ear and holds at least one first electromagnetic energy emitter 3111 (substantially located at an electrode position selected from the group consisting of AF3, AFz, F5, TP7, FC5, T7, and C5); and rear loop/portion 3102 which loops around the rear of the person's head from the location within 2″ of their ear and holds at least one second electromagnetic energy emitter 3112 (substantially located at an electrode position selected from the group consisting of O1, Oz, PO7, P7, TP7, and T7).

In an example, a neurostimulation device can comprise: a forward loop/portion which loops around the front of a person's head from a location within 2″ of their ear and holds at least one first electromagnetic energy emitter (substantially located at an electrode position selected from the group consisting of AF3, AFz, F5, TP7, FC5, T7, and C5); and rear loop/portion which loops around the rear of the person's head from the location within 2″ of their ear.

In an example, a neurostimulation device can comprise: a forward loop/portion which loops around the front of a person's head from a location within 2″ of their ear; and rear loop/portion which loops around the rear of the person's head from the location within 2″ of their ear and holds at least one second electromagnetic energy emitter (substantially located at an electrode position selected from the group consisting of O1, Oz, PO7, P7, TP7, and T7).

In an example, the forward loop/portion can have an undulating shape. In an example, the forward loop/portion can have a sinusoidal shape. In an example, the forward loop/portion can have a downward-facing concavity. In an example, the rear loop/portion can be substantially level/horizontal. In an example, the neurostimulation device can rest on a person's ears. In an example, the neurostimulation device can comprise an undulating ring which encircles a person's head. In an example, the neurostimulation device can comprise a partially-sinusoidal ring which encircles a person's head. In an example, the centroid of the forward loop/portion can be higher than the centroid of the rear loop/portion. In an example, the forward loop/portion can have an upward tilt as is extends forward from the person's ear to span the person's forehead.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 32 shows the left side of a neurostimulation device comprising: a sidepiece of an eyewear frame 3201 which holds at least one first electromagnetic energy emitter 3211 (substantially located at an electrode position selected from the group consisting of Nz, F9, FT9, and T7); an upward arm/portion 3202 which extends upward from the sidepiece at a location within 2″ of the person's ear and holds at least one second electromagnetic energy emitter 3212 (substantially located at an electrode position selected from the group consisting of T7 and C5); and a rear-ear arm/portion 3203 which curves around the rear of the person's ear and holds at least one third electromagnetic energy emitter 3213 (substantially located at an electrode position selected from the group consisting of P9, TP9, TP7, and T7).

In an example, the left side of a neurostimulation device can comprise: a sidepiece of an eyewear frame which holds at least one first electromagnetic energy emitter (substantially located at an electrode position selected from the group consisting of Nz, F9, FT9, and T7); an upward arm/portion which extends upward from the sidepiece at a location within 2″ of the person's ear; and a rear-ear arm/portion which curves around the rear of the person's ear.

In an example, the left side of a neurostimulation device can comprise: a sidepiece of an eyewear frame; an upward arm/portion which extends upward from the sidepiece at a location within 2″ of the person's ear and holds at least one second electromagnetic energy emitter (substantially located at an electrode position selected from the group consisting of T7 and C5); and a rear-ear arm/portion which curves around the rear of the person's ear.

In an example, the left side of a neurostimulation device can comprise: a sidepiece of an eyewear frame; an upward arm/portion which extends upward from the sidepiece at a location within 2″ of the person's ear; and a rear-ear arm/portion which curves around the rear of the person's ear and holds at least one third electromagnetic energy emitter (substantially located at an electrode position selected from the group consisting of P9, TP9, TP7, and T7).

In an example, the upward arm/portion can be arcuate. In an example, the upward arm/portion can have a rear-facing concavity. In an example, the upward arm/portion can extend upward to a location above the person's ear. In an example, the upward arm/portion can be have a first configuration in which it is aligned with the sidepiece and a second configuration in which it extends upward from the sidepiece. In an example, the upward arm/portion can be pivoted or rotated from its first configuration to its second configuration, or vice versa.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 33 shows the left side of a neurostimulation device comprising: an upper-rear loop 3301 which loops, from their ears, around an upper-rear portion of a person's head and holds at least one first electromagnetic energy emitter 3311 (substantially located at an electrode position selected from the group consisting of P7, P5, P3, P1, and Pz); a front ear arm/portion 3302 which curves around the front of the person's ear and holds at least one second electromagnetic energy emitter 3312 (substantially located at an electrode position selected from the group consisting of TP7, T9, T7); and a rear ear arm/portion 3303 which holds at least one third electromagnetic energy emitter 3313 (substantially located at an electrode position selected from the group consisting of P9, TP9, and TP7).

In an example, a neurostimulation device can comprise: an upper-rear loop which loops, from their ears, around an upper-rear portion of a person's head and holds at least one first electromagnetic energy emitter (substantially located at an electrode position selected from the group consisting of P7, P5, P3, P1, and Pz); a front ear arm/portion which curves around the front of the person's ear; and a rear ear arm/portion which curves around the rear of the person's ear.

In an example, a neurostimulation device can comprise: an upper-rear loop which loops, from their ears, around an upper-rear portion of a person's head; a front ear arm/portion which curves around the front of the person's ear and holds at least one second electromagnetic energy emitter (substantially located at an electrode position selected from the group consisting of TP7, T9, T7); and a rear ear arm/portion which curves around the rear of the person's ear.

In an example, a neurostimulation device can comprise: an upper-rear loop which loops, from their ears, around an upper-rear portion of a person's head; a front ear arm/portion which curves around the front of the person's ear; and a rear ear arm/portion which curves around the rear of the person's ear and holds at least one third electromagnetic energy emitter (substantially located at an electrode position selected from the group consisting of P9, TP9, and TP7).

In an example, the upper-rear loop can extend upward and rearward from a person's ear at an angle between 35 and 55 degrees relative a horizontal plane. In an example, the upper-rear loop can extend upward and rearward from a person's ear at an angle between 25 and 65 degrees relative a horizontal plane. In an example, the upper-rear loop can have a rear-facing concavity. In an example, the front ear arm/portion and the rear ear arm/portion can together span between 50% and 90% of the circumference of the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 34 shows the left side of a neurostimulation device comprising: an upper front loop 3401 which loops over the front half of the top of a person's head and holds at least one first electromagnetic energy emitter 3411 (substantially located at an electrode position selected from the group consisting of F9, F7, F5, FT9, FC3, FC1, FCz, T9, and T7); an upper rear loop 3402 which loops over the rear half of the top of a person's head and holds at least one second electromagnetic energy emitter 3412 (substantially located at an electrode position selected from the group consisting of P7, TP7, CP5, CP3, CP1, and CPz); and a mid-level arm/portion 3403 which connects the upper front loop to the upper rear loop, loops around the rear of the person's head, and holds at least one third electromagnetic energy emitter 3413 (substantially located at an electrode position selected from the group consisting of Iz, O1, Oz, PO7, P7, and TP7).

In an example, a neurostimulation device can comprise: an upper front loop which loops over the front half of the top of a person's head and holds at least one first electromagnetic energy emitter (substantially located at an electrode position selected from the group consisting of F9, F7, F5, FT9, FC3, FC1, FCz, T9, and T7); an upper rear loop which loops over the rear half of the top of a person's head; and a mid-level arm/portion which connects the upper front loop to the upper rear loop and also loops around the rear of the person's head.

In an example, a neurostimulation device can comprise: an upper front loop which loops over the front half of the top of a person's head; an upper rear loop which loops over the rear half of the top of a person's head and holds at least one second electromagnetic energy emitter (substantially located at an electrode position selected from the group consisting of P7, TP7, CP5, CP3, CP1, and CPz); and a mid-level arm/portion which connects the upper front loop to the upper rear loop and also loops around the rear of the person's head.

In an example, a neurostimulation device can comprise: an upper front loop which loops over the front half of the top of a person's head; an upper rear loop which loops over the rear half of the top of a person's head; and a mid-level arm/portion which connects the upper front loop to the upper rear loop, loops around the rear of the person's head, and holds at least one third electromagnetic energy emitter (substantially located at an electrode position selected from the group consisting of Iz, O1, Oz, PO7, P7, and TP7).

In an example, the upper front loop can have a forward-facing concavity. In an example, the upper rear loop can have a forward-facing concavity. In an example, the upper front loop can be undulating (e.g. sinusoidal). In an example, the upper rear loop can be undulating (e.g. sinusoidal). In an example, the neurostimulation device can rest on the person's ears. In an example, the mid-level arm/portion can be substantially horizontal. In an example, the upper front loop can be forward of a vertical plane which connects the person's ears. In an example, the upper front loop can be to the rear of a vertical plane which connects the person's ears. In an example, a first virtual plane which best fits the upper front loop and a second virtual plane which best fits the upper rear loops can be substantially parallel.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 35 shows the left side of a neurostimulation device comprising: an upper rear loop 3501 which loops, from a person's ears, around the rear half of the top of a person's head and holds at least one first electromagnetic energy emitter 3511 (substantially located at an electrode position selected from the group consisting of TP7, CP5, CP3, CP1, and CPz); a front ear arm/portion 3502 which extends forward from the person's ear and holds at least one second electromagnetic energy emitter 3512 (substantially located at an electrode position selected from the group consisting of TP7, T9, and T7); and a rear ear arm/portion 3503 which curves around the rear of the person's ear and holds at least one third electromagnetic energy emitter 3513 (substantially located at an electrode position selected from the group consisting of P9, TP9, and TP7).

In an example, a neurostimulation device can comprise: an upper rear loop which loops, from a person's ears, around the rear half of the top of a person's head and holds at least one first electromagnetic energy emitter (substantially located at an electrode position selected from the group consisting of TP7, CP5, CP3, CP1, and CPz); a front ear arm/portion which extends forward from the person's ear; and a rear ear arm/portion which curves around the rear of the person's ear.

In an example, a neurostimulation device can comprise: an upper rear loop which loops, from a person's ears, around the rear half of the top of a person's head; a front ear arm/portion which extends forward from the person's ear and holds at least one second electromagnetic energy emitter (substantially located at an electrode position selected from the group consisting of TP7, T9, and T7); and a rear ear arm/portion which curves around the rear of the person's ear.

In an example, a neurostimulation device can comprise: an upper rear loop which loops, from a person's ears, around the rear half of the top of a person's head; a front ear arm/portion which extends forward from the person's ear; and a rear ear arm/portion which curves around the rear of the person's ear and holds at least one third electromagnetic energy emitter (substantially located at an electrode position selected from the group consisting of P9, TP9, and TP7).

In an example, the upper rear loop can extend upward and rearward from a person's ear at an angle between 25 and 55 degrees relative to a horizontal plane. In an example, the upper rear loop can extend upward and rearward from a person's ear at an angle between 45 and 75 degrees relative to a horizontal plane. In an example, an upper rear loop can be undulating (e.g. sinusoidal). In an example, an upper rear loop can have a forward-facing concavity. In an example, a front ear arm/portion can be undulating (e.g. sinusoidal). In an example, a front ear arm/portion can extend forward in a direct anterior manner/direction from the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 36 shows the left side of a neurostimulation device comprising: a front arm/portion 3601 which loops around a person's forehead from the person's ears and holds at least one first electromagnetic energy emitter 3611 (substantially located at an electrode position selected from the group consisting of FPz, AF3, F5, FT7, T9, and T7); an upper rear arm/portion 3602 from loops around an upper rear portion of the person's head from the person's ears and holds at least one second electromagnetic energy emitter 3612 (substantially located at an electrode position selected from the group consisting of P7, P5, P3, P1, Pz, TP7 and T7); and a lower rear arm/portion 3603 which loops around a lower rear portion of the person's head and holds at least one third electromagnetic energy emitter 3613 (substantially located at an electrode position selected from the group consisting of P9, TP9, and TP7).

In an example, a neurostimulation device can comprise: a front arm/portion which loops around a person's forehead from the person's ears and holds at least one first electromagnetic energy emitter (substantially located at an electrode position selected from the group consisting of FPz, AF3, F5, FT7, T9, and T7); an upper rear arm/portion from loops around an upper rear portion of the person's head from the person's ears; and a lower rear arm/portion which loops around a lower rear portion of the person's head.

In an example, a neurostimulation device can comprise: a front arm/portion which loops around a person's forehead from the person's ears; an upper rear arm/portion from loops around an upper rear portion of the person's head from the person's ears and holds at least one second electromagnetic energy emitter (substantially located at an electrode position selected from the group consisting of P7, P5, P3, P1, Pz, TP7 and T7); and a lower rear arm/portion which loops around a lower rear portion of the person's head.

In an example, a neurostimulation device can comprise: a front arm/portion which loops around a person's forehead from the person's ears; an upper rear arm/portion from loops around an upper rear portion of the person's head from the person's ears; and a lower rear arm/portion which loops around a lower rear portion of the person's head and holds at least one third electromagnetic energy emitter (substantially located at an electrode position selected from the group consisting of P9, TP9, and TP7).

In an example, the front arm/portion and the lower rear arm/portion can together comprise an undulating (e.g. sinusoidal) ring which encircles the person's head. In an example, this ring can have a downward-facing concavity over the person's ear. In an example, the virtual plane which best fits this ring can intersect a horizontal plane (when the person is standing) at an angle between 5 and 45 degrees. In an example, the front arm/portion can have a downward-facing concavity. In an example, the lower rear arm/portion can have an upward-facing concavity. In an example, the upper rear arm/portion can have a forward-facing concavity.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

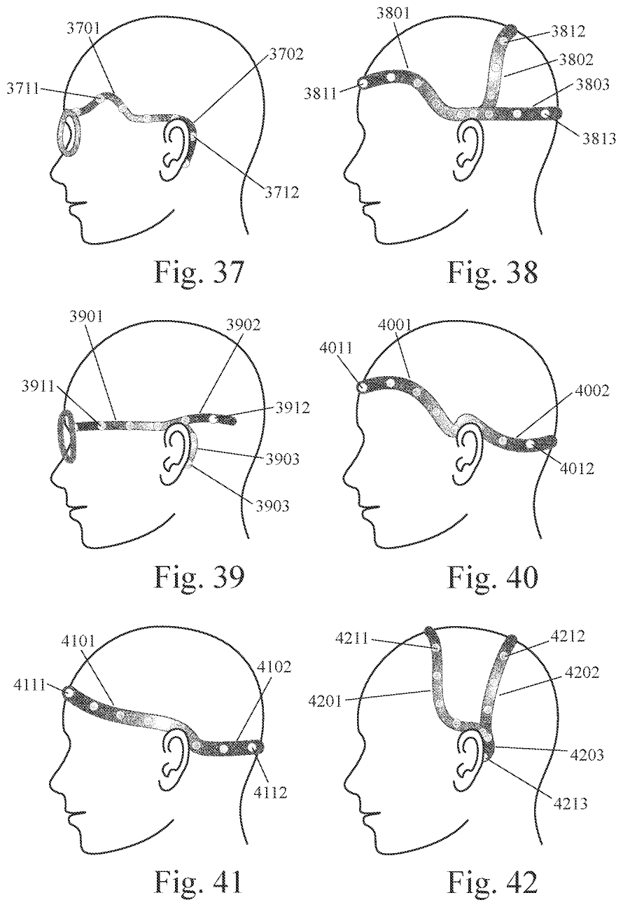

FIG. 37 shows the left side of an eyeglasses frame for sensing brain activity comprising: a sidepiece (e.g. sometimes called a “temple”) of the eyeglasses frame with an upward wave 3701 which holds at least one first electromagnetic energy emitter 3711 (substantially located at an electrode position selected from the group consisting of Nz, AF7, F9, F7, FT9, FT7, and T7) and a rear-ear portion 3702 which curves around the rear of the person's ear and holds at least one second electromagnetic energy emitter 3712 (substantially located at an electrode position selected from the group consisting of TP9, TP7, and T7).

In an example, the left side of an eyeglasses frame for sensing brain activity can comprise: a sidepiece of the eyeglasses frame with an upward wave which holds at least one first electromagnetic energy emitter (substantially located at an electrode position selected from the group consisting of Nz, AF7, F9, F7, FT9, FT7, and T7) and a rear-ear portion which curves around the rear of the person's ear.

In an example, the left side of an eyeglasses frame for sensing brain activity can comprise: a sidepiece of the eyeglasses frame with an upward wave and a rear-ear portion which curves around the rear of the person's ear and holds at least one second electromagnetic energy emitter (substantially located at an electrode position selected from the group consisting of TP9, TP7, and T7).

In an example, the upward wave of the sidepiece can be shaped like a conic section. In an example, the upward wave of the sidepiece can be a sinusoidal wave or a portion of a sinusoidal wave. In an example, the upward wave can extend onto (or over) a portion of the person's forehead and/or temple. In an example, the upward wave can extend inward (toward the person's head) as well as upward. In an example, the upward wave can hold an electromagnetic sensor in contact with the person's head. In an example, the upward wave can itself be electroconductive. In an example, the upward wave can be made from an electroconductive polymer. In an example, the upward wave can be made from an elastomeric polymer which is doped, impregnated, or coated with conductive material (such as silver or carbon particles).

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 38 shows the left side of a neurostimulation device comprising: a front arm/portion 3801 which loops around a person's forehead from the person's ears and holds at least one first electromagnetic energy emitter 3811 (substantially located at an electrode position selected from the group consisting of FPz, AF3, F5, F3, TP7, FT7, FC5, and T7); an upper arm/portion 3802 which loops over the person's head and holds at least one second electromagnetic energy emitter 3812 (substantially located at an electrode position selected from the group consisting of TP7, CP5, CP3, CP1, CPz); and a rear arm/portion 3803 which loops around the rear of the person's and holds at least one third electromagnetic energy emitter 3813 (substantially located at an electrode position selected from the group consisting of O1, Oz, PO7, P7, and TP7).

In an example, a neurostimulation device can comprise: a front arm/portion which loops around a person's forehead from the person's ears and holds at least one first electromagnetic energy emitter (substantially located at an electrode position selected from the group consisting of FPz, AF3, F5, F3, TP7, FT7, FC5, and T7); an upper arm/portion which loops over the person's head; and a rear arm/portion which loops around the rear of the person's head.

In an example, a neurostimulation device can comprise: a front arm/portion which loops around a person's forehead from the person's ears; an upper arm/portion which loops over the person's head and holds at least one second electromagnetic energy emitter (substantially located at an electrode position selected from the group consisting of TP7, CP5, CP3, CP1, CPz); and a rear arm/portion which loops around the rear of the person's head.

In an example, a neurostimulation device can comprise: a front arm/portion which loops around a person's forehead from the person's ears; an upper arm/portion which loops over the person's head; and a rear arm/portion which loops around the rear of the person's head and holds at least one third electromagnetic energy emitter (substantially located at an electrode position selected from the group consisting of O1, Oz, PO7, P7, and TP7).

In an example, the front arm/portion and the rear arm/portion can together comprise a ring which encircles the person's head. In an example, the front arm/portion and the rear arm/portion can together comprise an undulating ring which encircles the person's head. In an example, the front arm/portion can be higher than the rear arm/portion. In an example, the front arm/portion can have a downward-facing concavity. In an example, the rear arm/portion can be substantially level (e.g. horizontal when the person is standing up). In an example, the upper arm/portion can have a rear-facing concavity. In an example, the upper arm/portion can connect to the rear arm/portion at a location which is posterior to the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 39 shows the left side of a neurostimulation device comprising: eyeglasses with a sidepiece 3901 which holds at least one first electromagnetic energy emitter 3911 (substantially located at an electrode position selected from the group consisting of Nz, F9, FT9, and T7); a backward arm 3902, wherein the backward arm protrudes backward from the person's ear, but does not loop all the way around the rear of the person's head), and wherein the backward arm holds at least one second electromagnetic energy emitter 3912 (substantially located at an electrode position selected from the group consisting of PO7, P7, TP7, and T7); and a rear ear arm/portion 3903 which curves around the rear of the person's ear and holds at least one third electromagnetic energy emitter 3913 (substantially located at an electrode position selected from the group consisting of TP9, TP7, and T7).

In an example, the left side of a neurostimulation device can comprise: eyeglasses with a sidepiece which holds at least one first electromagnetic energy emitter (substantially located at an electrode position selected from the group consisting of Nz, F9, FT9, and T7); a backward arm, wherein the backward arm protrudes backward from the person's ear, but does not loop all the way around the rear of the person's head; and a rear ear arm/portion which curves around the rear of the person's ear.

In an example, the left side of a neurostimulation device can comprise: eyeglasses with a sidepiece; a backward arm, wherein the backward arm protrudes backward from the person's ear, but does not loop all the way around the rear of the person's head), and wherein the backward arm holds at least one second electromagnetic energy emitter (substantially located at an electrode position selected from the group consisting of PO7, P7, TP7, and T7); and a rear ear arm/portion which curves around the rear of the person's ear.

In an example, the left side of a neurostimulation device can comprise: eyeglasses with a sidepiece; a backward arm, wherein the backward arm protrudes backward from the person's ear, but does not loop all the way around the rear of the person's head), and wherein the backward arm; and a rear ear arm/portion which curves around the rear of the person's ear and holds at least one third electromagnetic energy emitter (substantially located at an electrode position selected from the group consisting of TP9, TP7, and T7).

In an example, the backward arm can have a length in the range of 1″ to 4.″ In an example, the backward arm can extend between 33% and 66% of the way from the person's ear to the back of their head. In an example, the backward arm can be substantially co-linear with the sidepiece.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 40 shows the left side of a neurostimulation device comprising: a forward loop 4001 which loops (from a person's ears) around the person's forehead and holds at least one first electromagnetic energy emitter 4011 (substantially located at an electrode position selected from the group consisting of FPz, AF3, F5, FT7, FC5, T9, and T7); and a rear loop 4002 which loops (from the person's ears) around the rear of a person's head and wherein this second arm/portion holds at least one second electromagnetic energy emitter 4012 (substantially located at an electrode position selected from the group consisting of Iz, P9, TP9, TP7, and T7).

In an example, a neurostimulation device can comprise: a forward loop which loops (from a person's ears) around the person's forehead and holds at least one first electromagnetic energy emitter (substantially located at an electrode position selected from the group consisting of FPz, AF3, F5, FT7, FC5, T9, and T7); and a rear loop which loops (from the person's ears) around the rear of a person's head.

In an example, a neurostimulation device can comprise: a forward loop which loops (from a person's ears) around the person's forehead; and a rear loop which loops (from the person's ears) around the rear of a person's head and wherein this second arm/portion holds at least one second electromagnetic energy emitter (substantially located at an electrode position selected from the group consisting of Iz, P9, TP9, TP7, and T7).

In an example, the forward loop and the rear loop can together comprise an undulating (e.g. sinusoidal) ring which encircles a person's head. In an example, the forward loop and the rear loop can together comprise an undulating ring which encircles a person's head with a downward-facing concavity over the person's ear. In an example, this ring can rest on a person's ears. In an example, the front loop can have a downward-facing concavity. In an example, the rear loop can have an upward-facing concavity. In an example, the front loop can be higher than the rear loop.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 41 shows the left side of a neurostimulation device comprising: a front arm/portion 4101 which spans from a person's ear to their forehead and holds at least one first electromagnetic energy emitter 4111 (substantially located at an electrode position selected from the group consisting of FP1, FPz, AF7, F7, TP7, FT7, and T7); and a rear arm/portion 4102 which spans from the person's ear to the rear of their head and holds at least one second electromagnetic energy emitter 4112 (substantially located at an electrode position selected from the group consisting of Iz, P9, TP9, TP7, and T7).

In an example, the left side of a neurostimulation device can comprise: a front arm/portion which spans from a person's ear to their forehead and holds at least one first electromagnetic energy emitter (substantially located at an electrode position selected from the group consisting of FP1, FPz, AF7, F7, TP7, FT7, and T7); and a rear arm/portion which spans from the person's ear to the rear of their head.

In an example, the left side of a neurostimulation device can comprise: a front arm/portion which spans from a person's ear to their forehead; and a rear arm/portion which spans from the person's ear to the rear of their head and holds at least one second electromagnetic energy emitter (substantially located at an electrode position selected from the group consisting of Iz, P9, TP9, TP7, and T7).

In an example, the front arm/portion and the rear arm/portion can together comprise a ring which encircles the person's head. In an example, this ring can be an undulating ring. In an example, this ring can have sinusoidal waves. In an example, this ring can rest on top of the person's ears. In an example, the front arm/portion can be higher than the rear arm/portion. In an example, the centroid of the front arm/portion can be higher than the centroid of the rear arm/portion. In an example, the front arm/portion can extend forward and upward from the top of the person's ear to their forehead. In an example, the rear arm/portion can extend backward from the top or middle of the person's ear to the rear of their head. In an example, the rear arm/portion can extend backward and downward from the top or middle of the person's ear to the rear of their head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 42 shows the left side of a neurostimulation device comprising: an upper front arm/portion 4201 which loops (from a person's ears) over the top of the front half of the person's head and holds at least one first electromagnetic energy emitter 4211 (substantially located at an electrode position selected from the group consisting of FT7, FC5, FC3, FC1, FCz, and T7); an upper rear arm/portion 4202 which loops (from the person's ears) over the top of the rear half of the person's head and holds at least one second electromagnetic energy emitter 4212 (substantially located at an electrode position selected from the group consisting of TP9, TP7, CP5, CP3, CP1, and CPz); and a rear ear arm/portion 4203 which curves around the rear of the person's ear and holds at least one third electromagnetic energy emitter 4213 (substantially located at an electrode position selected from the group consisting of TP9, TP7, and T7).

In an example, a neurostimulation device can comprise: an upper front arm/portion which loops (from a person's ears) over the top of the front half of the person's head and holds at least one first electromagnetic energy emitter (substantially located at an electrode position selected from the group consisting of FT7, FC5, FC3, FC1, FCz, and T7); an upper rear arm/portion which loops (from the person's ears) over the top of the rear half of the person's head; and a rear ear arm/portion which curves around the rear of the person's ear.

In an example, a neurostimulation device can comprise: an upper front arm/portion which loops (from a person's ears) over the top of the front half of the person's head; an upper rear arm/portion which loops (from the person's ears) over the top of the rear half of the person's head and holds at least one second electromagnetic energy emitter (substantially located at an electrode position selected from the group consisting of TP9, TP7, CP5, CP3, CP1, and CPz); and a rear ear arm/portion which curves around the rear of the person's ear.

In an example, a neurostimulation device can comprise: an upper front arm/portion which loops (from a person's ears) over the top of the front half of the person's head; an upper rear arm/portion which loops (from the person's ears) over the top of the rear half of the person's head; and a rear ear arm/portion which curves around the rear of the person's ear and holds at least one third electromagnetic energy emitter (substantially located at an electrode position selected from the group consisting of TP9, TP7, and T7).

In an example, this neurostimulation device can rest on (the tops of) the person's ears. In an example, the upper front arm/portion can have a rear-facing concavity. In an example, the upper rear arm/portion can have a rear-facing concavity. In an example, the upper front arm/portion and the upper rear arm/portion can diverge as they extend upward from a person's ear to loop over the top of the person's head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

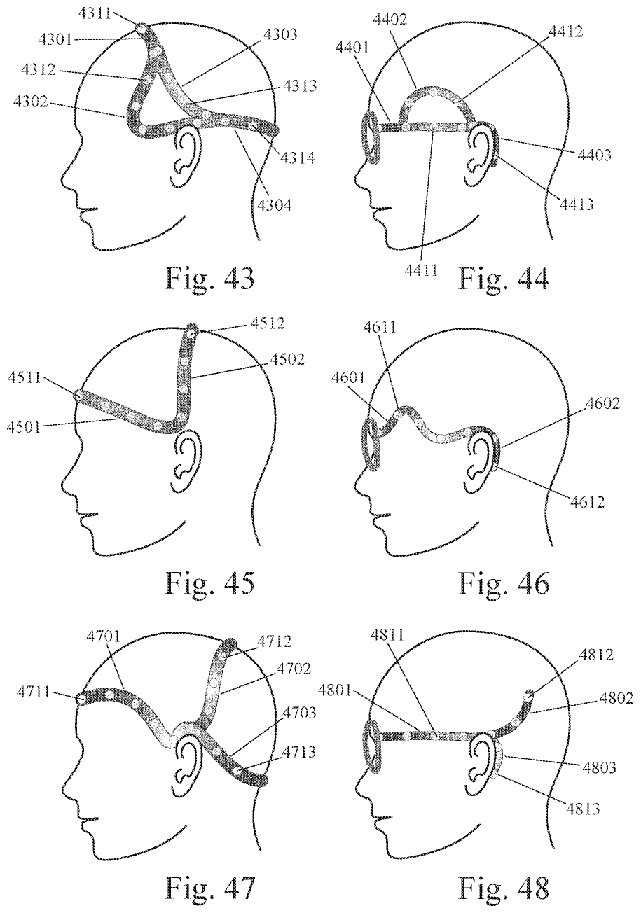

FIG. 43 shows the left side of a neurostimulation device comprising: an upper arm/portion 4301 which loops over the top of a person's head and holds at least one first electromagnetic energy emitter 4311 (substantially located at an electrode position selected from the group consisting of FC3, FC1, and FCz); a forward-middle arm/portion 4302 which holds at least one second electromagnetic energy emitter 4312 (substantially located at an electrode position selected from the group consisting of F9, F7, F5, TP7, FT9, FC3, and T7); a rear-middle arm/portion 4303 which holds at least one third electromagnetic energy emitter 4313 (substantially located at an electrode position selected from the group consisting of TP7, FC3, and C5); and a rear arm/portion 4304 which loops around the rear of the person's head and holds at least one fourth electromagnetic energy emitter 4314 (substantially located at an electrode position selected from the group consisting of Iz, O1, Oz, PO7, P7, and TP7).

In an example, a neurostimulation device can comprise: an upper arm/portion which loops over the top of a person's head and holds at least one first electromagnetic energy emitter (substantially located at an electrode position selected from the group consisting of FC3, FC1, and FCz); a forward-middle arm/portion; a rear-middle arm/portion; and a rear arm/portion which loops around the rear of the person's head.

In an example, a neurostimulation device can comprise: an upper arm/portion which loops over the top of a person's head; a forward-middle arm/portion which holds at least one second electromagnetic energy emitter (substantially located at an electrode position selected from the group consisting of F9, F7, F5, TP7, FT9, FC3, and T7); a rear-middle arm/portion; and a rear arm/portion which loops around the rear of the person's head.

In an example, a neurostimulation device can comprise: an upper arm/portion which loops over the top of a person's head; a forward-middle arm/portion; a rear-middle arm/portion which holds at least one third electromagnetic energy emitter (substantially located at an electrode position selected from the group consisting of TP7, FC3, and C5); and a rear arm/portion which loops around the rear of the person's head.

In an example, a neurostimulation device can comprise: an upper arm/portion which loops over the top of a person's head; a forward-middle arm/portion; a rear-middle arm/portion; and a rear arm/portion which loops around the rear of the person's head and holds at least one fourth electromagnetic energy emitter (substantially located at an electrode position selected from the group consisting of Iz, O1, Oz, PO7, P7, and TP7).

In an example: the forward-middle portion can connect the upper arm/portion and the rear arm/portion at first and second locations; and the rear-middle arm/portion can also connect the upper arm/portion and the rear arm/portion at the first and second locations. In an example, the forward-middle arm/portion can be anterior to the rear-middle arm/portion. In an example, the centroid of the forward-middle arm/portion can be anterior to the centroid of the rear-middle arm/portion. In an example, the left side of this neurostimulation device can be shaped like arcuate capital letter “A” which has been rotated counter-clockwise between 90 and 180 degrees. In an example, the upper, rear-middle, and rear arms/portions of this neurostimulation device can together create a shape with an upward-facing concavity. In an example, the upper, rear-middle, and rear arms/portions of this neurostimulation device can together create a shape with an upward-and-rearward-facing concavity. In an example, an opening between the forward-middle arm/portion and the rear-middle arm/portion can have a sinusoidal wave shape. In an example, an opening between the forward-middle arm/portion and the rear-middle arm/portion can have the shape of a single-phase sinusoidal wave.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 44 shows the left side of a neurostimulation device comprising: an eyewear sidepiece 4401 which holds at least one first electromagnetic energy emitter 4411 (substantially located at an electrode position selected from the group consisting of Nz, F9, FT9, and T7); an arc 4402 which extends upward from the eyewear sidepiece and holds at least one second electromagnetic energy emitter 4412 (substantially located at an electrode position selected from the group consisting of AF7, F5, FC5, and T7); and a rear ear arm/portion 4403 which curves around the rear of the person's ear and holds at least one third electromagnetic energy emitter 4413 (substantially located at an electrode position selected from the group consisting of P9, TP9, and TP7).

In an example, the left side of a neurostimulation device can comprise: an eyewear sidepiece which holds at least one first electromagnetic energy emitter (substantially located at an electrode position selected from the group consisting of Nz, F9, FT9, and T7); an arc which extends upward from the eyewear sidepiece; and a rear ear arm/portion which curves around the rear of the person's ear.

In an example, the left side of a neurostimulation device can comprise: an eyewear sidepiece; an arc which extends upward from the eyewear sidepiece and holds at least one second electromagnetic energy emitter (substantially located at an electrode position selected from the group consisting of AF7, F5, FC5, and T7); and a rear ear arm/portion which curves around the rear of the person's ear.

In an example, the left side of a neurostimulation device can comprise: an eyewear sidepiece; an arc which extends upward from the eyewear sidepiece; and a rear ear arm/portion which curves around the rear of the person's ear and holds at least one third electromagnetic energy emitter (substantially located at an electrode position selected from the group consisting of P9, TP9, and TP7).

In an example, the arc can have a conic section shape. In an example, the arc can have a sinusoidal wave shape. In an example, both front and the rear of the arc can be connected to the sidepiece. In an example, the arc can extend between ½″ and 3″ above the sidepiece. In an example, the height and/or curvature of the arc can be adjusted. In an example, the height and/or curvature of the arc can be changed by moving the location of the front end or the rear end of the arc relative to the sidepiece. In an example, the height and/or curvature of the arc can be changed by sliding the connection of the front end or the rear end of the arc along the sidepiece. In an example, the arc can have a downward-facing concavity. In an example, the front end of the arc can connect to the front third of the (length of the) sidepiece and the rear end of the arc can connect to the rear third of the (length of the) sidepiece.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 45 shows the left side of a neurostimulation device comprising: a front loop 4501 which loops around the front of the person's head (e.g. their forehead) and holds at least one first electromagnetic energy emitter 4511 (substantially located at an electrode position selected from the group consisting of FP1, FPz, AF7, F7, FT7, and T7); and an upper loop 4502 which loops over the top of the person's head (e.g. above their ears) and holds at least one second electromagnetic energy emitter 4512 (substantially located at an electrode position selected from the group consisting of T7, C5, C3, C1, and Cz).

In an example, a neurostimulation device can comprise: a front loop which loops around the front of the person's head (e.g. their forehead) and holds at least one first electromagnetic energy emitter (substantially located at an electrode position selected from the group consisting of FP1, FPz, AF7, F7, FT7, and T7); and an upper loop which loops over the top of the person's head (e.g. above their ears).

In an example, a neurostimulation device can comprise: a front loop which loops around the front of the person's head (e.g. their forehead); and an upper loop which loops over the top of the person's head (e.g. above their ears) and holds at least one second electromagnetic energy emitter (substantially located at an electrode position selected from the group consisting of T7, C5, C3, C1, and Cz).

In an example, the front loop can loop across and/or over the person's forehead. In an example, the front loop can loop across and/or over an upper front portion of the person's head. In an example, the front loop can be transparent or translucent. In an example, the front loop can tilt upward as it spans from a person's ear to the person's forehead. In an example, the front portion of the front loop can be at least 1″ higher than the rear portion of the front loop. In an example, the front loop can have a semicircular shape. In an example, the front loop can have a semielliptical shape. In an example, the front loop can have a downward-facing concavity. In an example, the front loop can have an upward-facing concavity. In an example, the upper loop can cross the top of a person's head in the vertical plane which connects a person's ears. In an example, this neurostimulation device can rest on the tops of a person's ears. In an example, the best-fitting plane for the front loop and the best-fitting plane for the upper loop can be substantially perpendicular to each other. In an example, the best-fitting plane for the front loop and the best-fitting plane for the upper loop can intersect at an angle between 70 and 90 degrees.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 46 shows the left side of a neurostimulation device comprising: an undulating (e.g. sinusoidal) eyeglasses sidepiece 4601 which holds at least one first electromagnetic energy emitter 4611 (substantially located at an electrode position selected from the group consisting of Nz, FP1, AF7, F9, F7, FT9, and T7); and a rear ear arm/portion 4602 which curves around the rear of a person's ear and holds at least one second electromagnetic energy emitter 4612 (substantially located at an electrode position selected from the group consisting of P9, TP9, and TP7).

In an example, the left side of a neurostimulation device can comprise: an undulating (e.g. sinusoidal) eyeglasses sidepiece which holds at least one first electromagnetic energy emitter (substantially located at an electrode position selected from the group consisting of Nz, FP1, AF7, F9, F7, FT9, and T7); and a rear ear arm/portion which curves around the rear of a person's ear.

In an example, the left side of a neurostimulation device can comprise: an undulating (e.g. sinusoidal) eyeglasses sidepiece; and a rear ear arm/portion which curves around the rear of a person's ear and holds at least one second electromagnetic energy emitter (substantially located at an electrode position selected from the group consisting of P9, TP9, and TP7).

In an example, an undulating eyeglasses sidepiece can have a sinusoidal shape. In an example, a sidepiece can have at least two undulations. In an example, a sidepiece can comprise a complete sinusoidal cycle shape (including both phases). In an example, the front half of an eyeglasses sidepiece can have a downward-facing concavity and the rear half of the sidepiece can have an upward-facing concavity. In an example, the front half of an eyeglasses sidepiece can have an upward (sinusoidal or conic section) curve which spans a portion of the person's forehead and/or temple. In an example, the front half of an eyeglasses sidepiece can curve inward (closer to the person's head) as well as upward. In an example, the front half of an eyeglasses sidepiece can include an arcuate portion (e.g. a wave or arc) which curves inward (closer to the person's head) as well as upward.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 47 shows the left side of a neurostimulation device comprising: a front arm/portion 4701 which extends forward onto the front of a person's head and holds at least one first electromagnetic energy emitter 4711 (substantially located at an electrode position selected from the group consisting of FPz, AF3, F5, FT7, T9, and T7); a upper arm/portion 4702 which extends upward onto the top of a person's head and holds at least one second electromagnetic energy emitter 4712 (substantially located at an electrode position selected from the group consisting of TP9, TP7, CP5, CP3, CP1, and CPz); a rear arm/portion 4703 which extends backward onto the rear of the person's head and holds at least one third electromagnetic energy emitter 4713 (substantially located at an electrode position selected from the group consisting of P9, TP9, and TP7).

In an example, the left side of a neurostimulation device can comprise: a front arm/portion which extends forward onto the front of a person's head and holds at least one first electromagnetic energy emitter (substantially located at an electrode position selected from the group consisting of FPz, AF3, F5, FT7, T9, and T7); a upper arm/portion which extends upward onto the top of a person's head; a rear arm/portion which extends backward onto the rear of the person's head.

In an example, the left side of a neurostimulation device can comprise: a front arm/portion which extends forward onto the front of a person's head; a upper arm/portion which extends upward onto the top of a person's head and holds at least one second electromagnetic energy emitter (substantially located at an electrode position selected from the group consisting of TP9, TP7, CP5, CP3, CP1, and CPz); a rear arm/portion which extends backward onto the rear of the person's head.

In an example, the left side of a neurostimulation device can comprise: a front arm/portion which extends forward onto the front of a person's head; a upper arm/portion which extends upward onto the top of a person's head; a rear arm/portion which extends backward onto the rear of the person's head and holds at least one third electromagnetic energy emitter (substantially located at an electrode position selected from the group consisting of P9, TP9, and TP7).

In an example, the front arm/portion can loop entirely around the front of the person's head (e.g. from one ear to the other). In an example, the upper arm/portion can loop entirely over the top of the person's head (e.g. from one ear to the other). In an example, the upper arm/portion can loop entirely around the rear of the person's head (e.g. from one ear to the other). In an example, the front arm/portion, the upper arm/portion, and the rear arm/portion can intersection and/or connect at a location (directly) above the person's ear. In an example, the neurostimulation device can rest on the tops of the person's ears. In an example, the front arm/portion and the rear arm/portion can together comprise a ring which encircles the person's head. In an example, the front arm/portion and the rear arm/portion can together comprise an undulating (e.g. sinusoidal) ring. In an example, the front arm/portion and the rear arm/portion can together comprise an undulating ring with downward-facing concavities over the person's ears. In an example, the front of this ring can be higher than the rear of this ring. In an example, the virtual plane which best fits this ring can intersect a horizontal plane when the person is standing up forming a forward-facing angle between 5 and 75 degrees.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 48 shows the left side of a neurostimulation device comprising: an eyeglasses sidepiece 4801 which holds at least one first electromagnetic energy emitter 4811 (substantially located at an electrode position selected from the group consisting of Nz, F9, FT9, and T7); an upward arm 4802 which extends upward and rearward from the person's ear and holds at least one second electromagnetic energy emitter 4812 (substantially located at an electrode position selected from the group consisting of P7, P5, P3, TP7, and T7); and a rear ear curve 4803 which curves around the rear of the person's ear and holds at least one third electromagnetic energy emitter 4813 (substantially located at an electrode position selected from the group consisting of P9, TP9, and TP7).

In an example, the left side of a neurostimulation device can comprise: an eyeglasses sidepiece which holds at least one first electromagnetic energy emitter (substantially located at an electrode position selected from the group consisting of Nz, F9, FT9, and T7); an upward arm which extends upward and rearward from the person's ear; and a rear ear curve which curves around the rear of the person's ear.

In an example, the left side of a neurostimulation device can comprise: an eyeglasses sidepiece; an upward arm which extends upward and rearward from the person's ear and holds at least one second electromagnetic energy emitter (substantially located at an electrode position selected from the group consisting of P7, P5, P3, TP7, and T7); and a rear ear curve which curves around the rear of the person's ear.

In an example, the left side of a neurostimulation device can comprise: an eyeglasses sidepiece; an upward arm which extends upward and rearward from the person's ear; and a rear ear curve which curves around the rear of the person's ear and holds at least one third electromagnetic energy emitter (substantially located at an electrode position selected from the group consisting of P9, TP9, and TP7).

In an example, the upward arm can have a length between 1″ and 4″. In an example, the upward arm can have a forward-facing concavity. In an example, the upward arm can have a forward-and-upward-facing concavity. In an example, the sidepiece and the upward arm can together have an upward-facing “hockey stick” shape. In an example, the sidepiece of an eyeglasses frame can bifurcate into an upward arm (which extends upward and rearward) and a rear ear curve (which curves around the rear of an ear). In an example, this bifurcation can occur within 1″ of the (top of the) person's ear. In an example, this bifurcation can comprise a “whale tail” shape.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

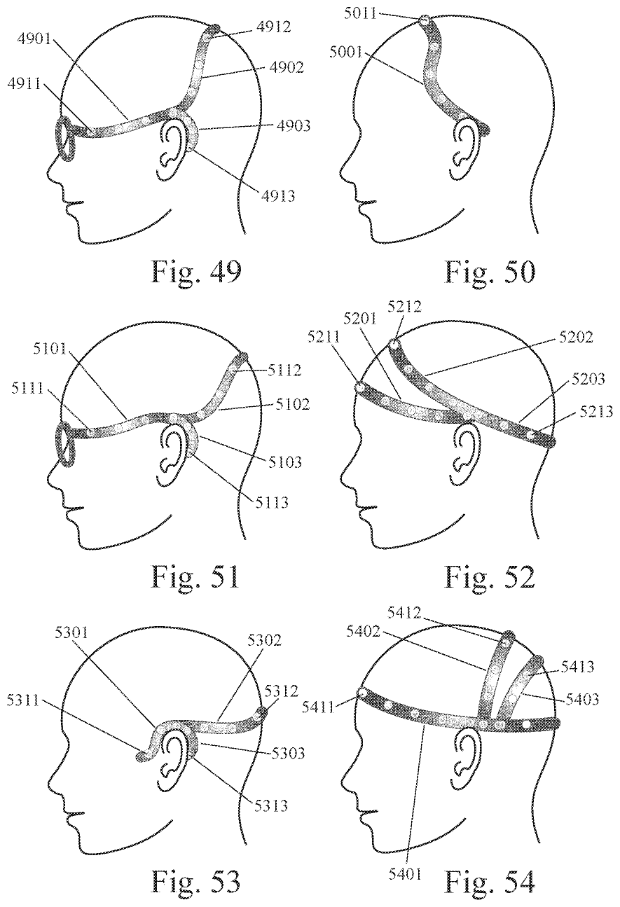

FIG. 49 shows the left side of a neurostimulation device comprising: an eyewear sidepiece 4901 which holds at least one first electromagnetic energy emitter 4911 (substantially located at an electrode position selected from the group consisting of Nz, F9, FT9, and T7); an upper loop 4902 which extends upward from the rear of the sidepiece, loops around the rear half of the top of the person's head, and holds at least one second electromagnetic energy emitter 4912 (substantially located at an electrode position selected from the group consisting of TP7, CP5, CP3, CP1, CPz, and T7); and a rear ear arm/portion 4903 which curves around the rear of the person's ear and holds at least one third electromagnetic energy emitter 4913 (substantially located at an electrode position selected from the group consisting of P9, TP9, and TP7).

In an example, a neurostimulation device can comprise: an eyewear sidepiece which holds at least one first electromagnetic energy emitter (substantially located at an electrode position selected from the group consisting of Nz, F9, FT9, and T7); an upper loop which extends upward from the rear of the sidepiece and loops around the rear half of the top of the person's head; and a rear ear arm/portion which curves around the rear of the person's ear.

In an example, a neurostimulation device can comprise: an eyewear sidepiece; an upper loop which extends upward from the rear of the sidepiece, loops around the rear half of the top of the person's head, and holds at least one second electromagnetic energy emitter (substantially located at an electrode position selected from the group consisting of TP7, CP5, CP3, CP1, CPz, and T7); and a rear ear arm/portion which curves around the rear of the person's ear.

In an example, a neurostimulation device can comprise: an eyewear sidepiece; an upper loop which extends upward from the rear of the sidepiece and loops around the rear half of the top of the person's head; and a rear ear arm/portion which curves around the rear of the person's ear and holds at least one third electromagnetic energy emitter (substantially located at an electrode position selected from the group consisting of P9, TP9, and TP7).

In an example, the eyewear sidepiece can have an upward-facing concavity. In an example, the upper loop can have a rear-facing concavity. In an example, the neurostimulation device can rest on the (tops of the) person's ears. In an example, the upper loop can be substantially perpendicular to the sidepiece. In an example, the upper loop can have an undulating shape.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 50 shows the left side of a neurostimulation device comprising: an arcuate hairband, tiara, or headband 5001 which loops over the top of a person's head from one ear to the other ear and holds at least one first electromagnetic energy emitter 5011 (substantially located at an electrode position selected from the group consisting of TP9, FT7, FC5, FC3, FC1, FCz, and T7).

In an example, a neurostimulation device can comprise: an arcuate hairband which loops over the top of a person's head from one ear to the other ear and holds at least one first electromagnetic energy emitter (substantially located at an electrode position selected from the group consisting of TP9, FT7, FC5, FC3, FC1, FCz, and T7). In an example, a neurostimulation device can comprise: an arcuate tiara which loops over the top of a person's head from one ear to the other ear and holds at least one first electromagnetic energy emitter (substantially located at an electrode position selected from the group consisting of TP9, FT7, FC5, FC3, FC1, FCz, and T7). In an example, a neurostimulation device can comprise: a semicircular headband which loops over the top of a person's head from one ear to the other ear and holds at least one first electromagnetic energy emitter (substantially located at an electrode position selected from the group consisting of TP9, FT7, FC5, FC3, FC1, FCz, and T7).

In an example, the neurostimulation device can rest on the (tops of the) person's ears. In an example, the neurostimulation device can further comprise projections, combs, and/or teeth which protrude between strands of the person's hair. In an example, the hairband, tiara, or headband can have a rear-facing convexity. In an example, the hairband, tiara, or headband can have a forward-facing convexity. In an example, the hairband, tiara, or headband can have a forward-facing convexity and a rear-facing convexity. In an example, the hairband, tiara, or headband can be undulating. In an example, the hairband, tiara, or headband can have a sinusoidal shape.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 51 shows the left side of a neurostimulation device comprising: an eyewear sidepiece (sometimes called a “temple”) 5101 which holds at least one first electromagnetic energy emitter 5111 (substantially located at an electrode position selected from the group consisting of Nz, F9, FT9, FT7, and T7); a loop 5102 which loops around the an upper rear portion of the person' head and holds at least one second electromagnetic energy emitter 5112 (substantially located at an electrode position selected from the group consisting of P7, P5, P3, P1, Pz, TP7, and T7); and a rear ear arm/portion 5103 which curves around the rear of the person's ear and holds at least one third electromagnetic energy emitter 5113 (substantially located at an electrode position selected from the group consisting of P9, TP9, and TP7).

In an example, the left side of a neurostimulation device can comprise: an eyewear sidepiece which holds at least one first electromagnetic energy emitter (substantially located at an electrode position selected from the group consisting of Nz, F9, FT9, FT7, and T7); a loop which loops around the an upper rear portion of the person' head; and a rear ear arm/portion which curves around the rear of the person's ear.

In an example, the left side of a neurostimulation device can comprise: an eyewear sidepiece; a loop which loops around the an upper rear portion of the person' head and holds at least one second electromagnetic energy emitter (substantially located at an electrode position selected from the group consisting of P7, P5, P3, P1, Pz, TP7, and T7); and a rear ear arm/portion which curves around the rear of the person's ear.

In an example, the left side of a neurostimulation device can comprise: an eyewear sidepiece; a loop which loops around the an upper rear portion of the person' head; and a rear ear arm/portion which curves around the rear of the person's ear and holds at least one third electromagnetic energy emitter (substantially located at an electrode position selected from the group consisting of P9, TP9, and TP7).

In an example, the loop can intersect the eyewear sidepiece at a forward-facing angle in the range of 100 to 150 degrees. In an example, the sidepiece can have a downward-facing concavity. In an example, the loop can have a rear-facing concavity. In an example, the loop can be elastic, stretchable, and/or expandable. In an example, the sidepiece and loop can be part of an undulating ring which encircles the person's head.

In an example, the right side of this neurostimulation device (not shown) can be laterally symmetric with respect to the left side. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 52 shows the left side of a neurostimulation device comprising: a lower front loop 5201 which loops around a person's forehead and holds at least one first electromagnetic energy emitter 5211 (substantially located at an electrode position selected from the group consisting of FP1, FPz, AF7, F7, FT7, and T7); an upper front loop 5202 which loops around the front of a person's head and holds at least one second electromagnetic energy emitter 5212 (substantially located at an electrode position selected from the group consisting of F3, F1, Fz, TP7, FC5, and C5), wherein the lower front loop is below the upper front loop; and a rear loop 5203 which loops around the rear of the person's head and holds at least one third electromagnetic energy emitter 5213 (substantially located at an electrode position selected from the group consisting of Iz, PO7, P7, and TP7).

In an example, a neurostimulation device can comprise: a lower front loop which loops around a person's forehead and holds at least one first electromagnetic energy emitter (substantially located at an electrode position selected from the group consisting of FP1, FPz, AF7, F7, FT7, and T7); an upper front loop which loops around the front of a person's head, wherein the lower front loop is below the upper front loop; and a rear loop which loops around the rear of the person's head.

In an example, a neurostimulation device can comprise: a lower front loop which loops around a person's forehead; an upper front loop which loops around the front of a person's head and holds at least one second electromagnetic energy emitter (substantially located at an electrode position selected from the group consisting of F3, F1, Fz, TP7, FC5, and C5), wherein the lower front loop is below the upper front loop; and a rear loop which loops around the rear of the person's head.

In an example, a neurostimulation device can comprise: a lower front loop which loops around a person's forehead; an upper front loop which loops around the front of a person's head, wherein the lower front loop is below the upper front loop; and a rear loop which loops around the rear of the person's head and holds at least one third electromagnetic energy emitter (substantially located at an electrode position selected from the group consisting of Iz, PO7, P7, and TP7).

In an example, the upper front loop and the rear loop can together comprise a ring which encircles the person's head. In an example, the front of this ring can be tilted upward relative to the rear of this ring. In an example the upper front loop and the rear loop can be linearly aligned where they connect to each other and/or merge into each other. In an example the upper front loop can be higher than the rear loop. In an example, this neurostimulation device can comprise a bifurcating ring which encircles a person's head. In an example, a ring can bifurcate into an upper front loop and a lower front loop from a location within 1″ of the person's ear. In an example, the upper front loop can comprise two segments which can be reversibly connected to each other. In an example, the lower front loop can comprise two segments which can be reversibly connected to each other. In an example, the rear loop can comprise two segments which can be reversibly connected to each other. In an example, this neurostimulation device can rest on the (tops of the) person's ears. In an example, the upper front loop can have an upward-facing convexity. In an example, the lower front loop can have an upward-facing convexity.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 53 shows the left side of a neurostimulation device comprising: a front ear arm/portion 5301 which extends 1″ to 3″ forward from the front of a person's ear and holds at least one first electromagnetic energy emitter 5311 (substantially located at an electrode position selected from the group consisting of TP7, T9, and T7); a rear loop 5302 which loops from the person's ear around the rear of the person's head and holds at least one second electromagnetic energy emitter 5312 (substantially located at an electrode position selected from the group consisting of O1, Oz, PO7, P7, TP7, and T7); and a rear ear arm/portion 5303 which curves around the rear of the person's ear and holds at least one third electromagnetic energy emitter 5313 (substantially located at an electrode position selected from the group consisting of P9, TP9, and TP7).

In an example, a neurostimulation device can comprise: a front ear arm/portion which extends 1″ to 3″ forward from the front of a person's ear and holds at least one first electromagnetic energy emitter (substantially located at an electrode position selected from the group consisting of TP7, T9, and T7); a rear loop which loops from the person's ear around the rear of the person's head; and a rear ear arm/portion which curves around the rear of the person's ear.

In an example, a neurostimulation device can comprise: a front ear arm/portion which extends 1″ to 3″ forward from the front of a person's ear; a rear loop which loops from the person's ear around the rear of the person's head and holds at least one second electromagnetic energy emitter (substantially located at an electrode position selected from the group consisting of O1, Oz, PO7, P7, TP7, and T7); and a rear ear arm/portion which curves around the rear of the person's ear.

In an example, a neurostimulation device can comprise: a front ear arm/portion which extends 1″ to 3″ forward from the front of a person's ear; a rear loop which loops from the person's ear around the rear of the person's head; and a rear ear arm/portion which curves around the rear of the person's ear and holds at least one third electromagnetic energy emitter (substantially located at an electrode position selected from the group consisting of P9, TP9, and TP7).

In an example, the rear loop can curve upward as it extends toward the rear of the person's head. In an example, the rear loop can have an undulating shape. In an example, the rear loop can have an upward-facing convexity. In an example, the rear loop can loop around the rear of the person's head at substantially the height of the top of the person's ear. In an example, the front ear arm/portion can extend forward along a height which is substantially the height of the middle of the person's ear. In an example, the front ear arm/portion can be arcuate. In an example, the front ear arm/portion can be sinusoidal.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 54 shows the left side of a neurostimulation device comprising: a ring (such as a headband) 5401 which encircles a person's head and holds at least one first electromagnetic energy emitter 5411 (substantially located at an electrode position selected from the group consisting of FP1, FPz, AF7, F7, TP7, FT7, T7, P7, P5, P3, P1, and Pz); a first upper loop 5402 which extends from the ring over the top of the person's head and holds at least one second electromagnetic energy emitter 5412 (substantially located at an electrode position selected from the group consisting of O1, Oz, PO7, P7); and a second upper loop 5403 which extends from the ring over the top of the person's head and holds at least one third electromagnetic energy emitter 5413 (substantially located at an electrode position selected from the group consisting of TP7, CP5, CP3, CP1, and CPz).

In an example, a neurostimulation device can comprise: a ring (such as a headband) which encircles a person's head and holds at least one first electromagnetic energy emitter (substantially located at an electrode position selected from the group consisting of FP1, FPz, AF7, F7, TP7, FT7, T7, P7, P5, P3, P1, and Pz); a first upper loop which extends from the ring over the top of the person's head; and a second upper loop which extends from the ring over the top of the person's head.

In an example, a neurostimulation device can comprise: a ring (such as a headband) which encircles a person's head; a first upper loop which extends from the ring over the top of the person's head and holds at least one second electromagnetic energy emitter (substantially located at an electrode position selected from the group consisting of O1, Oz, PO7, P7); and a second upper loop which extends from the ring over the top of the person's head.

In an example, a neurostimulation device can comprise: a ring (such as a headband) which encircles a person's head; a first upper loop which extends from the ring over the top of the person's head; and a second upper loop which extends from the ring over the top of the person's head and holds at least one third electromagnetic energy emitter (substantially located at an electrode position selected from the group consisting of TP7, CP5, CP3, CP1, and CPz).

In an example, the first upper loop can be anterior to the second upper loop. In an example, the first upper loop and the second upper loop can both loop over the rear half of the top of the person's head. In an example, the first upper loop can loop over the front half of the top of the person's head and the second upper loop can both loop over the rear half of the top of the person's head. In an example, the first upper loop can have a rear-facing concavity. In an example, the first upper loop can have a forward-facing concavity and the second upper loop can have a rear-facing concavity. In an example, the neurostimulation device can rest on the (tops of the) person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

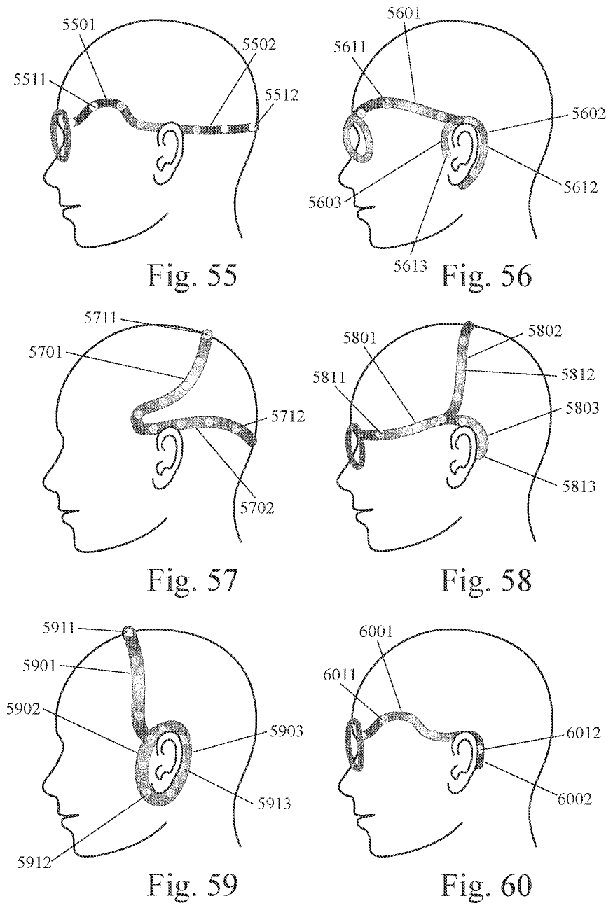

FIG. 55 shows the left side of a neurostimulation device comprising: an eyewear sidepiece (e.g. eyewear “temple”) 5501 which spans from a person's ear to the front of the person's face and holds at least one first electromagnetic energy emitter 5511 (substantially located at an electrode position selected from the group consisting of Nz, AF7, F7, FT9, FT7, and T7); and a rear loop 5502 which loops from the person's ear around the rear of the person's head and holds at least one second electromagnetic energy emitter 5512 (substantially located at an electrode position selected from the group consisting of Iz, O1, Oz, PO7, P9, P7, TP9, and TP7).

In an example, a neurostimulation device can comprise: an eyewear sidepiece which spans from a person's ear to the front of the person's face and holds at least one first electromagnetic energy emitter (substantially located at an electrode position selected from the group consisting of Nz, AF7, F7, FT9, FT7, and T7); and a rear loop which loops from the person's ear around the rear of the person's head.

In an example, a neurostimulation device can comprise: an eyewear sidepiece which spans from a person's ear to the front of the person's face; and a rear loop which loops from the person's ear around the rear of the person's head and holds at least one second electromagnetic energy emitter (substantially located at an electrode position selected from the group consisting of Iz, O1, Oz, PO7, P9, P7, TP9, and TP7).

In an example, the sidepiece and the rear loop can be a continuous member. In an example, the rear loop can be an extension of the sidepiece rearward of the person's ear. In an example, the rear loop can loop around the rear of the person's head at substantially the height as the top of the person's ear. In an example, the sidepiece can have a downward-facing concavity. In an example, the sidepiece can include an upward curve or arc. In an example, the middle of the sidepiece can have an upward (sinusoidal or conic section shaped) wave or undulation. In an example, the neurostimulation device can rest on the (tops of the) person's ears. In an example, the eyewear sidepiece can span from the person's ear to an eyewear frontpiece which holds two lenses. In an example, the eyewear can be a pair of eyeglasses.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 56 shows the left side of a neurostimulation device comprising: an eyewear sidepiece 5601 which holds at least one first electromagnetic energy emitter 5611 (substantially located at an electrode position selected from the group consisting of Nz, FP1, AF7, F7, FT7 and T7); a rear ear arm/portion 5602 which curves around the rear of a person's ear and holds at least one second electromagnetic energy emitter 5612 (substantially located at an electrode position selected from the group consisting of P9, TP9, TP7); and a front ear arm/portion 5603 which curves around the front of the person's ear and holds at least one third electromagnetic energy emitter 5613 (substantially located at an electrode position selected from the group consisting of TP7, T9, and T7).

In an example, the left side of a neurostimulation device can comprise: an eyewear sidepiece which holds at least one first electromagnetic energy emitter (substantially located at an electrode position selected from the group consisting of Nz, FP1, AF7, F7, FT7 and T7); a rear ear arm/portion which curves around the rear of a person's ear; and a front ear arm/portion which curves around the front of the person's ear.

In an example, the left side of a neurostimulation device can comprise: an eyewear sidepiece; a rear ear arm/portion which curves around the rear of a person's ear and holds at least one second electromagnetic energy emitter substantially located at an electrode position selected from the group consisting of P9, TP9, TP7); and a front ear arm/portion which curves around the front of the person's ear.

In an example, the left side of a neurostimulation device can comprise: an eyewear sidepiece; a rear ear arm/portion which curves around the rear of a person's ear; and a front ear arm/portion which curves around the front of the person's ear and holds at least one third electromagnetic energy emitter (substantially located at an electrode position selected from the group consisting of TP7, T9, and T7).

In an example, the rear ear arm/portion and the front ear arm/portion can together span between 50% and 90% of the circumference of a person's ear. In an example, the eyewear sidepiece can have a downward-facing concavity. In an example, the neurostimulation device can rest on the (tops of the) person's ears. In an example, the eyewear can be augmented reality eyewear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 57 shows the left side of a neurostimulation device comprising: an upper branch (or loop or arm) 5701 which loops from a position within 3″ of a person's ear around the rear half of the top of a person's head and holds at least one first electromagnetic energy emitter 5711 (substantially located at an electrode position selected from the group consisting of CP3, CP1, CPz, FT9, FT7, and C5); a lower branch (or loop or arm) 5702 which loops from the position within 3″ of the person's ear around the rear of the person's head and holds at least one second electromagnetic energy emitter 5712 (substantially located at an electrode position selected from the group consisting of Iz, O1, Oz, PO7, P7, TP7, FT9, and T7).

In an example, the left side of a neurostimulation device can comprise: an upper branch which loops from a position within 3″ of a person's ear around the rear half of the top of a person's head and holds at least one first electromagnetic energy emitter (substantially located at an electrode position selected from the group consisting of CP3, CP1, CPz, FT9, FT7, and C5); a lower branch which loops from the position within 3″ of the person's ear around the rear of the person's head.

In an example, the left side of a neurostimulation device can comprise: an upper branch which loops from a position within 3″ of a person's ear around the rear half of the top of a person's head; a lower branch which loops from the position within 3″ of the person's ear around the rear of the person's head and holds at least one second electromagnetic energy emitter (substantially located at an electrode position selected from the group consisting of Iz, O1, Oz, PO7, P7, TP7, FT9, and T7).

In an example, the upper branch and the lower branch can be part of the same continuous member. In an example, the upper branch can have an upward (or forward-upward) facing concavity. In an example, the lower branch can have a downward-facing concavity. In an example, the upper branch and the lower branch can diverge from each other as they span from the position near a person's ear to loop around different portions of the person's head. In an example, the upper branch and the lower branch as viewed from the left side of the person's head can have a shape like an open “bobby pin” or a cursive letter “v”. In an example, the neurostimulation device can rest on the (tops of the) person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 58 shows the left side of a neurostimulation device comprising: an eyewear sidepiece 5801 which holds at least one first electromagnetic energy emitter 5811 (substantially located at an electrode position selected from the group consisting of Nz, F9, FT9, FT7, and T7); an upper loop 5802 which loops over the top of the person's head and holds at least one second electromagnetic energy emitter 5812 (substantially located at an electrode position selected from the group consisting of T7, C5, C3, C1, and Cz); and a rear ear arm/portion 5803 which curves around the rear of the person's outer ear and holds at least one third electromagnetic energy emitter 5813 (substantially located at an electrode position selected from the group consisting of P9, TP9, TP7, and T7).

In an example, a neurostimulation device can comprise: an eyewear sidepiece which holds at least one first electromagnetic energy emitter (substantially located at an electrode position selected from the group consisting of Nz, F9, FT9, FT7, and T7); an upper loop which loops over the top of the person's head; and a rear ear arm/portion which curves around the rear of the person's outer ear.

In an example, a neurostimulation device can comprise: an eyewear sidepiece; an upper loop which loops over the top of the person's head and holds at least one second electromagnetic energy emitter (substantially located at an electrode position selected from the group consisting of T7, C5, C3, C1, and Cz); and a rear ear arm/portion which curves around the rear of the person's outer ear.

In an example, a neurostimulation device can comprise: an eyewear sidepiece; an upper loop which loops over the top of the person's head; and a rear ear arm/portion which curves around the rear of the person's outer ear and holds at least one third electromagnetic energy emitter (substantially located at an electrode position selected from the group consisting of P9, TP9, TP7, and T7).

In an example, the eyewear sidepiece can be arcuate. In an example, the eyewear sidepiece can be undulating (e.g. sinusoidal). In an example, the eyewear sidepiece can have a downward-facing concavity. In an example, the eyewear sidepiece can have an upward arc or wave. In an example, the middle of the eyewear sidepiece can have an upward arc or wave. In an example, the neurostimulation device can rest on the (tops of the) person's ears. In an example, the eyewear sidepiece can bifurcate into the upper loop and the rear ear arm/portion within 2″ of the person's ear. In an example, the upper loop can have a rear-facing concavity. In an example, the upper loop can have a forward-facing concavity. In an example, the upper loop can be (sinusoidally) undulating. In an example, the upper loop can be elastic, stretchable, and/or expandable.

In an example, the right side of this neurostimulation device (not shown) can be laterally symmetric with respect to the left side. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 59 shows the left side of a neurostimulation device comprising: an upper loop 5901 which loops over the top of a person's and holds at least one first electromagnetic energy emitter 5911 (substantially located at an electrode position selected from the group consisting of FT7, FC5, FC3, FC1, FCz, T9, and T7); a front ear arm/portion 5902 which curves around the front of a person's ear and holds at least one second electromagnetic energy emitter 5912 (substantially located at an electrode position selected from the group consisting of T9 and T7); and a rear ear arm/portion 5903 which curves around the rear of a person's ear and holds at least one third electromagnetic energy emitter 5913 (substantially located at an electrode position selected from the group consisting of P9, TP9, TP7, and T7).

In an example, a neurostimulation device can comprise: an upper loop which loops over the top of a person's and holds at least one first electromagnetic energy emitter (substantially located at an electrode position selected from the group consisting of FT7, FC5, FC3, FC1, FCz, T9, and T7); a front ear arm/portion which curves around the front of a person's ear; and a rear ear arm/portion which curves around the rear of a person's ear.

In an example, a neurostimulation device can comprise: an upper loop which loops over the top of a person's; a front ear arm/portion which curves around the front of a person's ear and holds at least one second electromagnetic energy emitter (substantially located at an electrode position selected from the group consisting of T9 and T7); and a rear ear arm/portion which curves around the rear of a person's ear.

In an example, a neurostimulation device can comprise: an upper loop which loops over the top of a person's; a front ear arm/portion which curves around the front of a person's ear; and a rear ear arm/portion which curves around the rear of a person's ear and holds at least one third electromagnetic energy emitter (substantially located at an electrode position selected from the group consisting of P9, TP9, TP7, and T7).

In an example, the front ear arm/portion and the rear ear arm/portion can be portions of the same continuous member which encircles a person's ear. In an example, the front ear arm/portion and the rear ear arm/portion can together comprise a ring which encircles a person's ear. In an example, the front ear arm/portion and the rear ear arm/portion can together comprise a ring around a person's outer ear. In an example, this neurostimulation device can comprise a set of headphones. In an example, the upper loop can be undulating and/or sinusoidal.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 60 shows the left side of a neurostimulation device comprising: an eyewear sidepiece 6001 which holds at least one first electromagnetic energy emitter 6011 (substantially located at an electrode position selected from the group consisting of Nz, AF7, F7, FT9, FT7, T9 and T7); and a rear ear arm/portion 6002 which curves around the back of a person's ear and holds at least one second electromagnetic energy emitter 6012 (substantially located at an electrode position selected from the group consisting of P9, TP9, TP7, and T7).

In an example, a neurostimulation device can comprise: an eyewear sidepiece which holds at least one first electromagnetic energy emitter (substantially located at an electrode position selected from the group consisting of Nz, AF7, F7, FT9, FT7, T9 and T7); and a rear ear arm/portion which curves around the back of a person's ear.

In an example, a neurostimulation device can comprise: an eyewear sidepiece; and a rear ear arm/portion which curves around the back of a person's ear and holds at least one second electromagnetic energy emitter (substantially located at an electrode position selected from the group consisting of P9, TP9, TP7, and T7).

In an example the rear ear arm/portion can be a continuous extension of the eyewear sidepiece. In an example, the eyewear sidepiece and the rear ear arm/portion can both be part of the same continuous member. In an example, the eyewear sidepiece can have a downward-facing concavity. In an example, the eyewear sidepiece can have an upward wave or curve. In an example, there can be an upward wave or curve in the middle of the sidepiece. In an example, the sidepiece can be undulating and/or sinusoidal. In an example, the middle of the eyewear sidepiece can curve inward (toward the person's head) as well as upward. In an example, the front of the eyewear sidepiece can curve inward (toward the person's head) as well as upward. In an example, the eyewear sidepiece can further comprise compressible foam on the body-facing side of the sidepiece. In an example, the eyewear sidepiece can further comprise a spring on the body-facing side of the sidepiece. In an example, the eyewear sidepiece can further comprise an inflatable compartment on the body-facing side of the sidepiece.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 61 shows the left side of a neurostimulation device comprising: an eyeglasses frame sidepiece (e.g. “temple”) 6101 which holds at least one first electromagnetic energy emitter 6111 (substantially located at an electrode position selected from the group consisting of Nz, F9, FT9, and T7); an upward arm 6102 which extends upward from the sidepiece and holds at least one second electromagnetic energy emitter 6112 (substantially located at an electrode position selected from the group consisting of FT9, FT7, and FC5); and a rear ear arm/portion 6103 which curves around the rear of the person's ear and holds at least one third electromagnetic energy emitter 6113 (substantially located at an electrode position selected from the group consisting of P9, TP9, and TP7).

In an example, the left side of a neurostimulation device can comprise: an eyeglasses frame sidepiece (e.g. “temple”) which holds at least one first electromagnetic energy emitter (substantially located at an electrode position selected from the group consisting of Nz, F9, FT9, and T7); an upward arm which extends upward from the sidepiece; and a rear ear arm/portion which curves around the rear of the person's ear.

In an example, the left side of a neurostimulation device can comprise: an eyeglasses frame sidepiece (e.g. “temple”); an upward arm which extends upward from the sidepiece and holds at least one second electromagnetic energy emitter (substantially located at an electrode position selected from the group consisting of FT9, FT7, and FC5); and a rear ear arm/portion which curves around the rear of the person's ear.

In an example, the left side of a neurostimulation device can comprise: an eyeglasses frame sidepiece (e.g. “temple”); and an upward arm which extends upward from the sidepiece and holds at least one second electromagnetic energy emitter (substantially located at an electrode position selected from the group consisting of FT9, FT7, and FC5).

In an example, the left side of a neurostimulation device can comprise: an eyeglasses frame sidepiece (e.g. “temple”); an upward arm which extends upward from the sidepiece; and a rear ear arm/portion which curves around the rear of the person's ear and holds at least one third electromagnetic energy emitter (substantially located at an electrode position selected from the group consisting of P9, TP9, and TP7).

In an example, the upward arm can pivot or rotate up or down. In an example, the upward arm can have a first configuration in which is it substantially aligned with the sidepiece, have a second configuration in which it is substantially perpendicular to the sidepiece, and can be pivoted or rotated from its first configuration to its second. In an example, the upward arm can have a first configuration in which is it less visible, have a second configuration in which it is more visible, and can be pivoted or rotated from its first configuration to its second. In an example, the upward arm can have a forward-facing concavity. In an example, the upward arm can have a rear-facing concavity. In an example, the upward arm can extend upward from the middle (third) portion of the eyeglasses frame sidepiece. In an example, the upward arm can be between 1″ and 3″ in length.

In an example, the right side of this neurostimulation device (not shown) can be laterally symmetric with respect to the left side. In an example, arms and/or portions of this neurostimulation device can be elastic, stretchable, and/or expandable. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

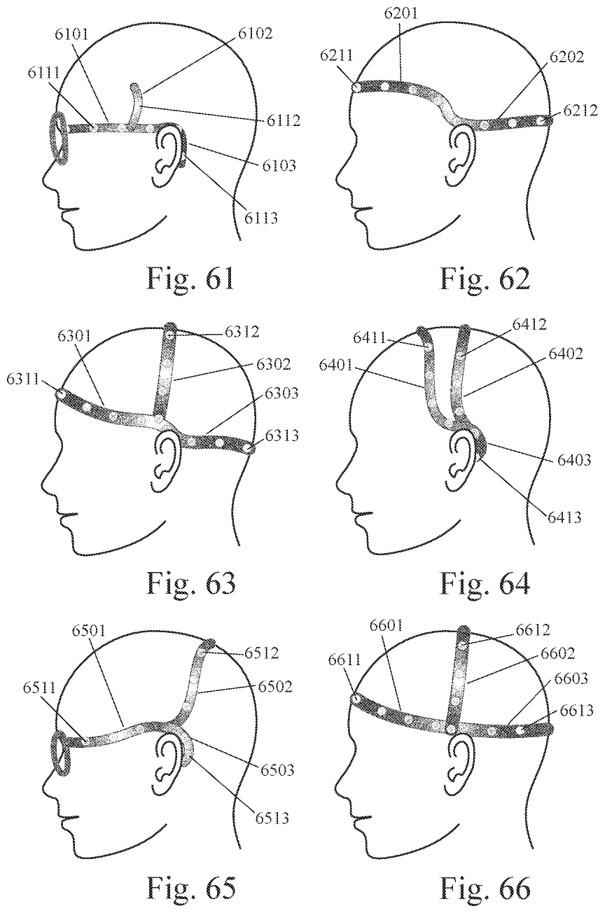

FIG. 62 shows the left side of a neurostimulation device comprising: a front loop 6201 which extends forward from a person's ear, loops around the front of the person's head, and holds at least one first electromagnetic energy emitter 6211 (substantially located at an electrode position selected from the group consisting of FPz, AF3, F5, FC5, and T7); and a rear loop 6202 which extends backward from the person's ear, loops around the rear of the person's head, and holds at least one second electromagnetic energy emitter 6212 (substantially located at an electrode position selected from the group consisting of Iz, O1, Oz, PO7, P7, TP7, and T7).

In an example, a neurostimulation device can comprise: a front loop which extends forward from a person's ear, loops around the front of the person's head, and holds at least one first electromagnetic energy emitter (substantially located at an electrode position selected from the group consisting of FPz, AF3, F5, FC5, and T7); and a rear loop which extends backward from the person's ear and loops around the rear of the person's head.

In an example, a neurostimulation device can comprise: a front loop which extends forward from a person's ear and loops around the front of the person's head; and a rear loop which extends backward from the person's ear, loops around the rear of the person's head, and holds at least one second electromagnetic energy emitter (substantially located at an electrode position selected from the group consisting of Iz, O1, Oz, PO7, P7, TP7, and T7).

In an example, the front loop and the rear loop can be portions of the same continuous member. In an example, the front loop and the rear loop can be part of an arcuate ring which encircles the person's head. In an example, the front loop can be higher than the rear loop. In an example, the front loop can be substantially level, the rear loop can be substantially level, and the front loop can be higher than the rear loop. In an example, the neurostimulation device can rest on the (tops of the) person's ears. In an example, the front loop can loop across the person's forehead. In an example, the front loop can have a downward-facing concavity. In an example, the rear loop can have an upward-facing concavity. In an example, the centroid of the front loop can be higher than the centroid of the rear loop (which the person is standing up). In an example, the front loop and the rear loop can together comprise an undulating ring which encircles the person's head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 63 shows the left side of a neurostimulation device comprising: a front arm/portion 6301 which loops around the front of a person's head and holds at least one first electromagnetic energy emitter 6311 (substantially located at an electrode position selected from the group consisting of FP1, FPz, AF7, F7, FT7, and T7); an upper arm/portion 6302 which loops over the top of the person's head and holds at least one second electromagnetic energy emitter 6312 (substantially located at an electrode position selected from the group consisting of T7, C5, C3, C1, and Cz); and a rear arm/portion 6303 which loops around the rear of the person's head and holds at least one third electromagnetic energy emitter 6313 (substantially located at an electrode position selected from the group consisting of Iz, P9, TP9, and T7).

In an example, a neurostimulation device can comprise: a front arm/portion which loops around the front of a person's head and holds at least one first electromagnetic energy emitter (substantially located at an electrode position selected from the group consisting of FP1, FPz, AF7, F7, FT7, and T7); an upper arm/portion which loops over the top of the person's head; and a rear arm/portion which loops around the rear of the person's head.

In an example, a neurostimulation device can comprise: a front arm/portion which loops around the front of a person's head; an upper arm/portion which loops over the top of the person's head and holds at least one second electromagnetic energy emitter (substantially located at an electrode position selected from the group consisting of T7, C5, C3, C1, and Cz); and a rear arm/portion which loops around the rear of the person's head.

In an example, a neurostimulation device can comprise: a front arm/portion which loops around the front of a person's head; an upper arm/portion which loops over the top of the person's head; and a rear arm/portion which loops around the rear of the person's head and holds at least one third electromagnetic energy emitter (substantially located at an electrode position selected from the group consisting of Iz, P9, TP9, and T7).

In an example, the front arm/portion and the rear arm/portion together can comprise a ring which encircles the person's head. In an example, the front arm/portion and the rear arm/portion together can comprise an undulating ring which encircles the person's head. In an example, the upper arm/portion can be substantially perpendicular to this ring. In an example, the centroid of the front arm/portion can be higher than the centroid of the rear arm/portion when the person is standing up. In an example, this ring can have a forward-upward tilt. In an example, this neurostimulation device can rest on the (tops of the) person's ears. In an example, the front arm/portion can have an upward-facing concavity. In an example, the front arm/portion can span the person's forehead.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 64 shows the left side of a neurostimulation device comprising: a first upper loop 6401 which loops over the top of a person's head and holds at least one first electromagnetic energy emitter 6411 (substantially located at an electrode position selected from the group consisting of FT7, FC5, FC3, FC1, FCz, and T7); a second upper loops 6402 which loops over the top of the person's head and hold at least one second electromagnetic energy emitter 6412 (substantially located at an electrode position selected from the group consisting of TP7, C5, C3, C1, and Cz), wherein the first upper loop is closer to the front of the person's head than the second upper loop; and rear ear arm/portion 6403 which curves around the rear of the person's ear and holds at least one third electromagnetic energy emitter 6413 (substantially located at an electrode position selected from the group consisting of P9, TP9, and TP7).

In an example, a neurostimulation device can comprise: a first upper loop which loops over the top of a person's head and holds at least one first electromagnetic energy emitter (substantially located at an electrode position selected from the group consisting of FT7, FC5, FC3, FC1, FCz, and T7); a second upper loops which loops over the top of the person's head, wherein the first upper loop is closer to the front of the person's head than the second upper loop; and rear ear arm/portion which curves around the rear of the person's ear.

In an example, a neurostimulation device can comprise: a first upper loop which loops over the top of a person's head; a second upper loops which loops over the top of the person's head and holds at least one second electromagnetic energy emitter (substantially located at an electrode position selected from the group consisting of TP7, C5, C3, C1, and Cz), wherein the first upper loop is closer to the front of the person's head than the second upper loop; and rear ear arm/portion which curves around the rear of the person's ear.

In an example, a neurostimulation device can comprise: a first upper loop which loops over the top of a person's head; a second upper loops which loops over the top of the person's head, wherein the first upper loop is closer to the front of the person's head than the second upper loop; and rear ear arm/portion which curves around the rear of the person's ear and holds at least one third electromagnetic energy emitter (substantially located at an electrode position selected from the group consisting of P9, TP9, and TP7).

In an example, the first upper loop and the second upper loop can connect at a location within 2″ of a person's ear. In an example, the first upper loop can have a rear-facing concavity. In an example, the first upper loop can be undulating and/or sinusoidal. In an example, the neurostimulation device can rest on the (tops of the) person's ears. In an example, the second upper loop can be substantially in a virtual vertical plane which connects the person's ears. In an example, the second upper loop can span the middle of the top of the person's head and the first upper loop can span the front-half of the top of the person's head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 65 shows the left side of a neurostimulation device comprising: an eyewear sidepiece 6501 which holds at least one first electromagnetic energy emitter 6511 (substantially located at an electrode position selected from the group consisting of Nz, F9, TP7, FT9, FT7, and T7); a rear-upper loop 6502 which extends rearward and upward from the rear of the sidepiece, loops over the rear half of the top of the person's head, and holds at least one second electromagnetic energy emitter 6512 (substantially located at an electrode position selected from the group consisting of TP7, CP5, CP3, CP1, CPz, and T7); and a rear ear arm/portion 6503 which curves around the rear of the person's ear and holds at least one third electromagnetic energy emitter 6513 (substantially located at an electrode position selected from the group consisting of P9, TP9, and TP7).

In an example, a neurostimulation device can comprise: an eyewear sidepiece which holds at least one first electromagnetic energy emitter (substantially located at an electrode position selected from the group consisting of Nz, F9, TP7, FT9, FT7, and T7); a rear-upper loop which extends rearward and upward from the rear of the sidepiece and loops over the rear half of the top of the person's head; and a rear ear arm/portion which curves around the rear of the person's ear.

In an example, a neurostimulation device can comprise: an eyewear sidepiece; a rear-upper loop which extends rearward and upward from the rear of the sidepiece, loops over the rear half of the top of the person's head, and holds at least one second electromagnetic energy emitter (substantially located at an electrode position selected from the group consisting of TP7, CP5, CP3, CP1, CPz, and T7); and a rear ear arm/portion which curves around the rear of the person's ear.

In an example, a neurostimulation device can comprise: an eyewear sidepiece; a rear-upper loop which extends rearward and upward from the rear of the sidepiece and loops over the rear half of the top of the person's head; and a rear ear arm/portion which curves around the rear of the person's ear and holds at least one third electromagnetic energy emitter (substantially located at an electrode position selected from the group consisting of P9, TP9, and TP7).

In an example, the neurostimulation device can rest on the (tops of the) person's ears. In an example, the sidepiece can be undulating and/or sinusoidal. In an example, the sidepiece can have a downward-facing concavity. In an example, the rear-upper loop can be undulating and/or sinusoidal. In an example, read-upper loop can have a rearward-facing concavity. In an example, the sidepiece and the rear-upper loop can diverge (directly) above the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 66 shows the left side of a neurostimulation device comprising: a front arm/portion 6601 which loops around the front of a person' head and holds at least one first electromagnetic energy emitter 6611 (substantially located at an electrode position selected from the group consisting of FP1, FPz, AF7, F7, FT7, and T7); an upper arm/portion 6602 which loops over the top of the person's head and holds at least one second electromagnetic energy emitter 6612 (substantially located at an electrode position selected from the group consisting of T7, C5, C3, C1, and Cz); a rear arm/portion 6603 which loops around the rear of the person's head and holds at least one third electromagnetic energy emitter 6613 (substantially located at an electrode position selected from the group consisting of O1, Oz, PO7, P7, TP7, and T7).

In an example, a neurostimulation device can comprise: a front arm/portion which loops around the front of a person' head and holds at least one first electromagnetic energy emitter (substantially located at an electrode position selected from the group consisting of FP1, FPz, AF7, F7, FT7, and T7); an upper arm/portion which loops over the top of the person's head; a rear arm/portion which loops around the rear of the person's head.

In an example, a neurostimulation device can comprise: a front arm/portion which loops around the front of a person' head; an upper arm/portion which loops over the top of the person's head and holds at least one second electromagnetic energy emitter (substantially located at an electrode position selected from the group consisting of T7, C5, C3, C1, and Cz); a rear arm/portion which loops around the rear of the person's head.

In an example, a neurostimulation device can comprise: a front arm/portion which loops around the front of a person' head; an upper arm/portion which loops over the top of the person's head; a rear arm/portion which loops around the rear of the person's head and holds at least one third electromagnetic energy emitter (substantially located at an electrode position selected from the group consisting of O1, Oz, PO7, P7, TP7, and T7).

In an example, the front arm/portion and the rear arm/portion can be part of the same continuous member which encircles a person's head. In an example, the front arm/portion and the rear arm/portion can together comprise a ring which encircles a person's head. In an example, the front of the ring can be higher than the rear of the ring. In an example a virtual plane which best fits the ring can intersect a horizontal plane when the person is standing up, forming a forward-facing angle between 5 and 75 degrees. In an example, the front arm/portion can further comprise two reversibly-connectable straps or bands. In an example, the rear arm/portion can further comprise two reversibly-connectable straps or bands. In an example, the upper arm/portion can further comprise two reversibly-connectable straps or bands. In an example, the front, rear, and upper arms/portions can be connected to each other (directly) above the person's ear. In an example, the neurostimulation device can rest on (the tops of) the person's ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

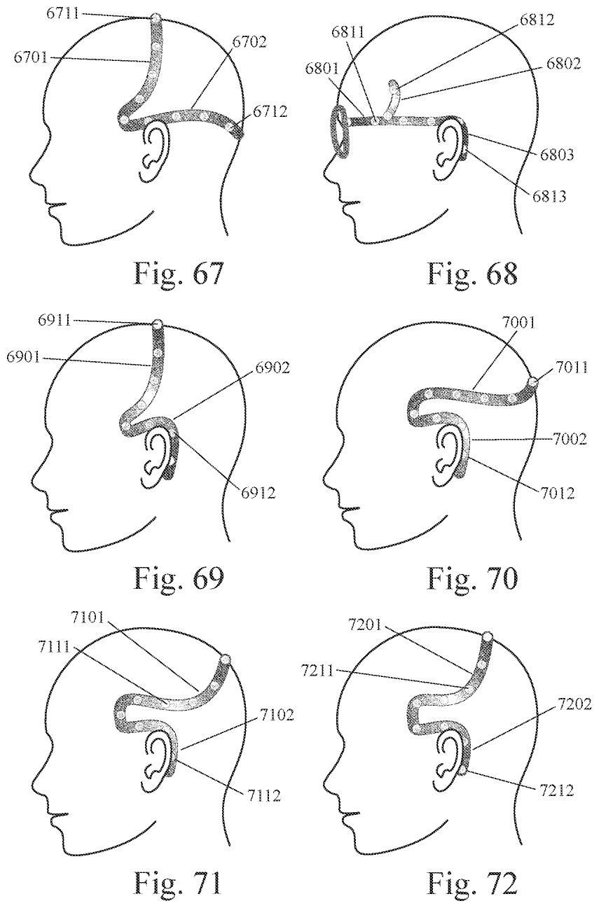

FIG. 67 shows the left side of a neurostimulation device comprising: an upper arm/portion 6701 which loops over the top of a person's head and holds at least one first electromagnetic energy emitter 6711 (substantially located at an electrode position selected from the group consisting of FT9, FT7, T9, T7, C5, C3, C1, and Cz); and a rear arm/portion 6702 which loops around the rear of the person's head and holds at least one second electromagnetic energy emitter 6712 (substantially located at an electrode position selected from the group consisting of Iz, O1, Oz, PO7, P7, TP7, and T7).

In an example, a neurostimulation device can comprise: an upper arm/portion which loops over the top of a person's head and holds at least one first electromagnetic energy emitter (substantially located at an electrode position selected from the group consisting of FT9, FT7, T9, T7, C5, C3, C1, and Cz); and a rear arm/portion which loops around the rear of the person's head.

In an example, a neurostimulation device can comprise: an upper arm/portion which loops over the top of a person's head; and a rear arm/portion which loops around the rear of the person's head and holds at least one second electromagnetic energy emitter (substantially located at an electrode position selected from the group consisting of Iz, O1, Oz, PO7, P7, TP7, and T7).

In an example, the upper arm/portion can further comprise two reversibly-connectible straps or bands. In an example, the rear arm/portion can further comprise two reversibly-connectible straps or bands. In an example, the upper arm/portion can have a forward-facing concavity. In an example, the rear arm/portion can have a downward-facing concavity. In an example, the neurostimulation device can rest on the (tops of the) person's ears. In an example, the virtual plane which best fits the upper arm/portion can be substantially perpendicular to the virtual plane which best fits the rear arm/portion. In an example, the virtual plane which best fits the upper arm/portion can be substantially vertical when the person is standing up. In an example, the virtual plane which best fits the upper arm/portion can be substantially co-planar with the vertical plane which best connects the person's ears.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 68 shows the left side of eyewear for sensing brain activity comprising: a sidepiece 6801 which holds at least one first electromagnetic energy emitter 6811 (substantially located at an electrode position selected from the group consisting of Nz, F9, FT9, and T7); an upward arm 6802 which extends upward from the sidepiece and holds at least one second electromagnetic energy emitter 6812 (substantially located at an electrode position selected from the group consisting of P9, TP9, and TP7); and a rear ear arm 6803 which curves around the rear of the person's ear and holds at least one third electromagnetic energy emitter 6813.

In an example, eyewear for sensing brain activity can comprise: a sidepiece which holds at least one first electromagnetic energy emitter (substantially located at an electrode position selected from the group consisting of Nz, F9, FT9, and T7); an upward arm which extends upward from the sidepiece; and a rear ear arm which curves around the rear of the person's ear and holds at least one third electromagnetic energy emitter.

In an example, eyewear for sensing brain activity can comprise: a sidepiece; an upward arm which extends upward from the sidepiece and holds at least one second electromagnetic energy emitter (substantially located at an electrode position selected from the group consisting of P9, TP9, and TP7); and a rear ear arm which curves around the rear of the person's ear and holds at least one third electromagnetic energy emitter.

In an example, the upward arm can pivot or rotate from a first configuration in which it is lower (and in substantial alignment with the sidepiece) and a second configuration in which it is raised (and substantially perpendicular to the side piece). In an example, the upward arm can be arcuate. In an example, the upward arm can have a forward-facing concavity. In an example, the upward arm can be connected to the middle third of the sidepiece. In an example, the upward arm can be 1″ to 3″ in length. In an example, the upward arm can bend toward the person's head as is extends upward from the sidepiece. In an example, the upward can further comprise a spring, compressible foam, or an inflatable compartment.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 69 shows the left side of a neurostimulation device comprising: an upper portion 6901 which extends upward from a person's ear, loops over the top of the person's head, and holds at least one first electromagnetic energy emitter 6911 (substantially located at an electrode position selected from the group consisting of FT9, FT7, T7, C5, C3, C1, and Cz); and a lower portion 6902 which curves around the rear of a person's ear and holds at least one second electromagnetic energy emitter 6912 (substantially located at an electrode position selected from the group consisting of P9, TP9, and TP7).

In an example, a neurostimulation device can comprise: an upper portion which extends upward from a person's ear, loops over the top of the person's head, and holds at least one first electromagnetic energy emitter (substantially located at an electrode position selected from the group consisting of FT9, FT7, T7, C5, C3, C1, and Cz); and a lower portion which curves around the rear of a person's ear.

In an example, a neurostimulation device can comprise: an upper portion which extends upward from a person's ear and loops over the top of the person's head; and a lower portion which curves around the rear of a person's ear and holds at least one second electromagnetic energy emitter (substantially located at an electrode position selected from the group consisting of P9, TP9, and TP7).

In an example, the upper portion can be arcuate. In an example, the upper portion can be undulating and/or sinusoidal. In an example, the upper portion can have a forward-facing concavity. In example, the upper portion can loop over the top of a person's head in substantially the virtual vertical plane which connects the person's ears. In an example, the neurostimulation device can rest on the (tops of the) person's ears. In an example, the lower portion can curve around the top and rear of the person's ear. In an example, the bottom portion can have a forward-facing concavity. In an example, the bottom portion can have a forward-and-downward-facing concavity. In an example, the neurostimulation device can have a forward curve or bulge where the upper and lower portions are connected to each other. In an example, the upper and lower portions can both be part of the same continuous band or strip. In an example, the virtual plane which best fits the overall neurostimulation device can be substantially vertical when the person is standing up.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 70 shows the left side of a neurostimulation device comprising: an upper portion 7001 which loops around the rear of a person's head from a location above and forward of a person's ear and which holds at least one first electromagnetic energy emitter 7011 (substantially located at an electrode position selected from the group consisting of PO3, POz, P5, CP5, FT7, FC5, T7, and C5); and lower portion 7002 which curves around the person's ear from the location above and forward of a person's ear and which holds at least one second electromagnetic energy emitter 7012 (substantially located at an electrode position selected from the group consisting of P9, TP9, TP7, and T7).

In an example, a neurostimulation device can comprise: an upper portion 7001 which loops around the rear of a person's head from a location above and forward of a person's ear and which holds at least one first electromagnetic energy emitter 7011 (substantially located at an electrode position selected from the group consisting of PO3, POz, P5, CP5, FT7, FC5, T7, and C5); and lower portion 7002 which curves around the person's ear from the location above and forward of a person's ear.

In an example, a neurostimulation device can comprise: an upper portion 7001 which loops around the rear of a person's head from a location above and forward of a person's ear; and lower portion 7002 which curves around the person's ear from the location above and forward of a person's ear and which holds at least one second electromagnetic energy emitter 7012 (substantially located at an electrode position selected from the group consisting of P9, TP9, TP7, and T7).

In an example, the upper portion and the lower portion can both be part of the same continuous band or strap. In an example, the location above and forward of the person's ear can be between ½″ and 3″ above the top of the person's ear and between 1″ and 4″ in front of the top of the person's ear. In an example, the upper portion can be substantially level. In an example, the upper portion can have an upward-facing concavity. In an example, the upper portion can loop around a central portion the rear of the person's head. In an example, the lower portion can curve around the rear of the person's ear. In an example, the neurostimulation device can rest on the (tops of the) person's ears. In an example, the overall neurostimulation device, including the upper and lower portions, can have a rear-facing concavity which is located (directly) over the person's ear. In an example, the overall neurostimulation device can be undulating and/or sinusoidal. In an example, the middle of the left side of the upper portion can be directly above the person's left ear.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 71 shows the left side of a neurostimulation device comprising: a upper portion 7101 which extends rearward and upward from a location above and forward of a person's ear, which loops over the rear half of the top of the person's head, and which holds at least one first electromagnetic energy emitter 7111 (substantially located at an electrode position selected from the group consisting of P5, P3, P1, Pz, CP5, FT7, FC5, T7, and C5); and a lower portion 7102 which extends downward and rearward from the location, which curves around the person's ear, and which holds at least one second electromagnetic energy emitter 7112 (substantially located at an electrode position selected from the group consisting of P9, TP9, TP7, and T7).

In an example, a neurostimulation device can comprise: a upper portion 7101 which extends rearward and upward from a location above and forward of a person's ear, which loops over the rear half of the top of the person's head, and which holds at least one first electromagnetic energy emitter 7111 (substantially located at an electrode position selected from the group consisting of P5, P3, P1, Pz, CP5, FT7, FC5, T7, and C5); and a lower portion 7102 which extends downward and rearward from the location and which curves around the person's ear.

In an example, a neurostimulation device can comprise: a upper portion 7101 which extends rearward and upward from a location above and forward of a person's ear, and which loops over the rear half of the top of the person's head; and a lower portion 7102 which extends downward and rearward from the location, which curves around the person's ear, and which holds at least one second electromagnetic energy emitter 7112 (substantially located at an electrode position selected from the group consisting of P9, TP9, TP7, and T7).

In an example, the upper portion and the lower portion can both be part of the same continuous member. In an example, the neurostimulation device can have a rear-facing concavity which is directly above the person's ear. In an example, the neurostimulation device can have a forward curve or bulge above and forward of the person's ear. In an example, the upper portion can have a forward-and-upward-facing concavity. In an example, the neurostimulation device can be undulating and/or sinusoidal. In an example, the neurostimulation device can be worn by Delenn and no one would notice. In an example, the upper and lower portions can be connected at a location over the person' temple. In an example, the upper and lower portions can be connected at a location within 1″ of the person' temple. In an example, the most-forward part of the neurostimulation device can be within 1″ of the person's temple.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

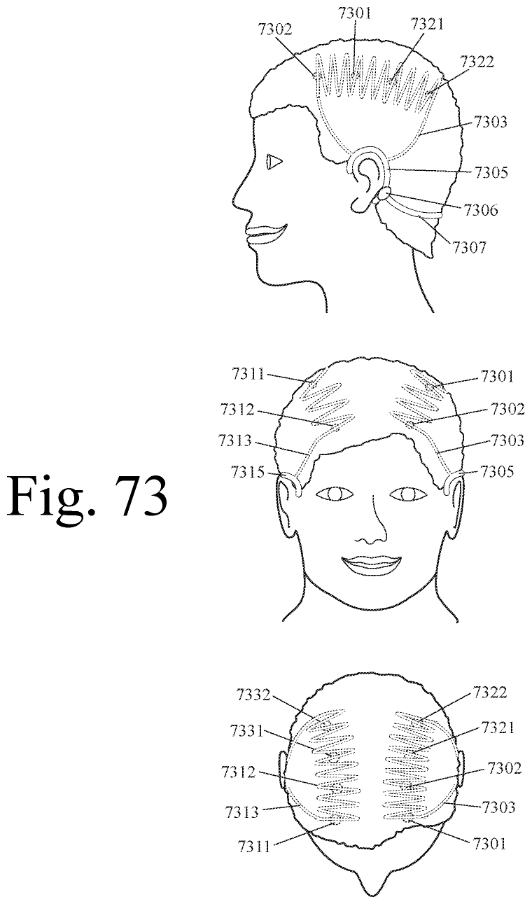

FIG. 72 shows the left side of a neurostimulation device comprising: an upper arcuate portion 7201 with a rear-facing concavity which extends upward from a person's ear, loops over the top of their head, and holds at least one first electromagnetic energy emitter 7211 (substantially located at an electrode position selected from the group consisting of CP3, CP1, CPz, FT9, FT7, FC5, T7, and C5); and a lower arcuate portion 7202 which curves around the rear of the person's ear and holds at least one second electromagnetic energy emitter 7212 (substantially located at an electrode position selected from the group consisting of P9, TP9, TP7, and T7).

In an example, a neurostimulation device can comprise: an upper arcuate portion with a rear-facing concavity which extends upward from a person's ear, loops over the top of their head, and holds at least one first electromagnetic energy emitter (substantially located at an electrode position selected from the group consisting of CP3, CP1, CPz, FT9, FT7, FC5, T7, and C5); and a lower arcuate portion which curves around the rear of the person's ear.

In an example, a neurostimulation device can comprise: an upper arcuate portion with a rear-facing concavity which extends upward from a person's ear and loops over the top of their head; and a lower arcuate portion which curves around the rear of the person's ear and holds at least one second electromagnetic energy emitter (substantially located at an electrode position selected from the group consisting of P9, TP9, TP7, and T7).

In an example, the upper arcuate portion and the lower arcuate portion can both be part of the same continuous band or strap. In an example, the left side of the neurostimulation device overall (including both upper and lower portions) can have a rear-facing concavity which is located directly above the person's left ear. In an example, the neurostimulation device can rest on the (tops of the) person's ears. In an example, the most forward part of the neurostimulation device can be located over the person's temple. In an example, the most forward part of the neurostimulation device can be located within 1″ of the person's temple. In an example, the neurostimulation device can curve upwards around the rear of the person's ear, curve forward and upward to the person's temple, and then curve upward and rearward to span the rear half of the top of the person's head.

In an example, the right side of this neurostimulation device (not shown) can be laterally symmetric with respect to the left side. In an example, arms and/or portions of this neurostimulation device can be elastic, stretchable, and/or expandable. In an example, arms and/or portions of this neurostimulation device can be pivoted or rotated around their points of intersection. In an example, one or more electromagnetic energy emitters can be attached to the arms and/or portions of this neurostimulation device. In an example, electroconductive areas of arms and/or portions of this neurostimulation device can comprise electromagnetic energy emitters. In an example, portions of this neurostimulation device can be transparent. Variations discussed elsewhere in this or priority-linked disclosures can also be applied to this example.

FIG. 73 shows another example of neurostimulation device. This example has prongs and/or teeth which slide into or under a person's hair. The top third of FIG. 73 shows this neurostimulation device looking at the side of a person's head. The middle third of FIG. 73 shows this neurostimulation device looking at the front of a person's head. The bottom third of FIG. 73 shows this neurostimulation device looking down at the top of a person's head.

As shown in the top third of FIG. 73, this neurostimulation device comprises: an arcuate frame which is configured to be worn on a person's head, wherein this arcuate frame further comprises: a left ear loop 7305 which is configured to curve around the person's left ear; a posterior loop 7307 which is connected to the left ear loop 7305, where this posterior loop 7307 is configured to curve around a posterior portion of a person's head; a left upward-extending member 7303 which is configured to loop upward toward the top of the person's head from the left ear loop 7305 and then back downward to left ear loop 7305, wherein this left upward-extending member has multiple prongs and/or teeth, wherein this left upward-extending member has a first configuration in which its upper-most portion is a first distance from the top of the person's head, wherein this left upward-extending member has a second configuration in which its upper-most portion is a second distance from the top of the person's head, wherein the second distance is less than the first distance, and wherein the prongs and/or teeth are configured to slide into or under hair in the second configuration; one or more left-side electromagnetic energy emitters, 7301, 7302, 7321, and 7322; and a left-side housing 7306 which contains a power source, a data processing unit, and a data transmitter and/or receiver.

The middle third and bottom third of FIG. 73 show this same neurostimulation device from a frontal face perspective and a top-down perspective, respectively. Right-side components include: a right ear loop 7315 which is configured to curve around the person's right ear; a right upward-extending member 7313 which is configured to loop upward toward the top of the person's head from the right ear loop 7315 and then back downward to right ear loop 7315, wherein this right upward-extending member has multiple prongs and/or teeth, wherein this right upward-extending member has a first configuration in which its upper-most portion is a first distance from the top of the person's head, wherein this right upward-extending member has a second configuration in which its upper-most portion is a second distance from the top of the person's head, wherein the second distance is less than the first distance, and wherein the prongs and/or teeth are configured to slide into or under hair in the second configuration; and one or more right-side electromagnetic energy emitters, 7311, 7312, 7331, and 7332. In this example, the upper tips of right and left upward-extending members are not connected or attached to each other, even in the second configuration.

In an example, a neurostimulation device can comprise: a wearable band which is configured to encircle a person's head at an anterior acute angle in the range of 0 to 45 degrees with respect to a horizontal plane when the person's head is upright, wherein the band has prongs or teeth which protrude into and/or under the person's hair; and a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy emitters are made from an elastic polymer.

In an example, a neurostimulation device can comprise: a wearable band which is configured to encircle a person's head at an anterior acute angle in the range of 0 to 45 degrees with respect to a horizontal plane when the person's head is upright; and a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy emitters are made from a silicone material which has been impregnated, doped, coated, or embedded with metal; and wherein one or more of the electromagnetic energy emitters have prongs or teeth which protrude into and/or under the person's hair.

In an example, a neurostimulation device can comprise: a wearable band which is configured to encircle a person's head at an anterior acute angle in the range of 0 to 45 degrees with respect to a horizontal plane when the person's head is upright; and a plurality of electromagnetic energy emitters on the band; wherein one or more of the electromagnetic energy emitters are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy emitters are made from a silicone material which has been impregnated, doped, coated, or embedded with carbon; and wherein one or more of the electromagnetic energy emitters have prongs or teeth which protrude into and/or under the person's hair. Variations discussed elsewhere in this or priority-linked disclosures can also be applied to this example.

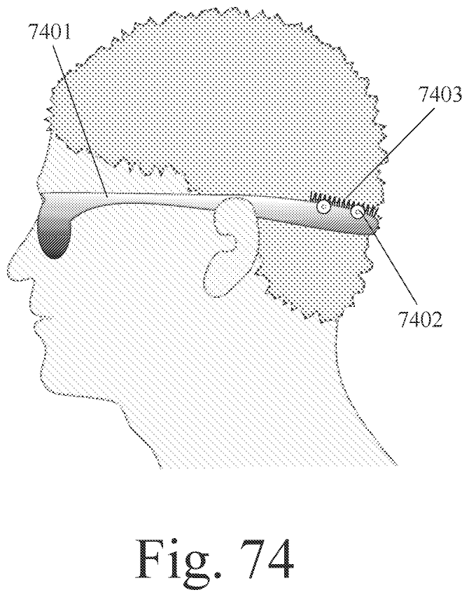

FIG. 74 shows a left-side view of another example of how this invention can be embodied in a neurostimulation device. In this example, this invention is embodied as a hybrid wearable EEG monitor with an integrated eyewear frame. In this example, a neurostimulation device comprises: an eyewear frame 7401 with a rear loop which loops around the rear of a person's head; a plurality of electromagnetic energy emitters (including 7402) which are held against the rear of the person's head by the eyewear frame; and prongs and/or teeth 7403 which protrude into and/or under the person's hair. In an example, the prongs and/or teeth which protrude into and/or under the person's hair can enable better electromagnetic communication between the emitters and the person's head. In an example, the electromagnetic energy emitters can be EEG emitters. In an example, this neurostimulation device can further comprise a power source, a data processor, and a wireless data transmitter. In an example, this neurostimulation device can further comprise a camera.

In this example, the anterior portion of the neurostimulation device serves to hold eyewear lenses and the posterior portion of the neurostimulation device loops completely around the back of the person's head. In this example, the front of this neurostimulation device rests on the bridge of the person's nose and the sides of this neurostimulation device rest on the person's ears. In this example, the anterior and posterior portions of this neurostimulation device form a continuous ring and/or band which encircles the person's head at a relatively-constant level which is just above the person's nose and ears. In this example, the eyewear portion of this neurostimulation device has lenses, but no display screen. In another example, the eyewear portion of this neurostimulation device may have a display screen, but no lenses. In another example, the eyewear portion of this neurostimulation device may have both a display screen and lenses. In an example, the eyewear portion of this neurostimulation device can serve as an augmented reality interface.

In an example, a neurostimulation device can comprise: eyewear which is configured to be worn on a person's head; one or more electrodes or other brain activity emitters which are configured by the eyewear to be less than one inch from the surface of the person's head; prongs and/or teeth which protrude into and/or under the person's hair; a mobile power source and/or power transducer; a data processor; and a data transmitting member. In an example, a neurostimulation device can comprise eyewear with a frame which encircles a person's head at an anterior acute angle in the range of 0 to 45 degrees with respect to a horizontal plane (when the person's head is upright) and a plurality of electromagnetic energy emitters on the eyewear frame. The electromagnetic energy emitters can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz. In an example, the electromagnetic energy emitters can be made from a silicone material (such as polydimethylsiloxane [PDMS]) which has been impregnated, doped, coated, or embedded with conductive material (such as silver or carbon) in order to provide consistent but comfortable contact with the person's head. In an example, this neurostimulation device can further comprise prongs and/or teeth which protrude from the ring and/or band into and/or under the person's hair to enable. This can enable good electromagnetic communication with the person's head at locations on the person's head which are covered by hair. Variations discussed elsewhere in this or priority-linked disclosures can also be applied to this example.

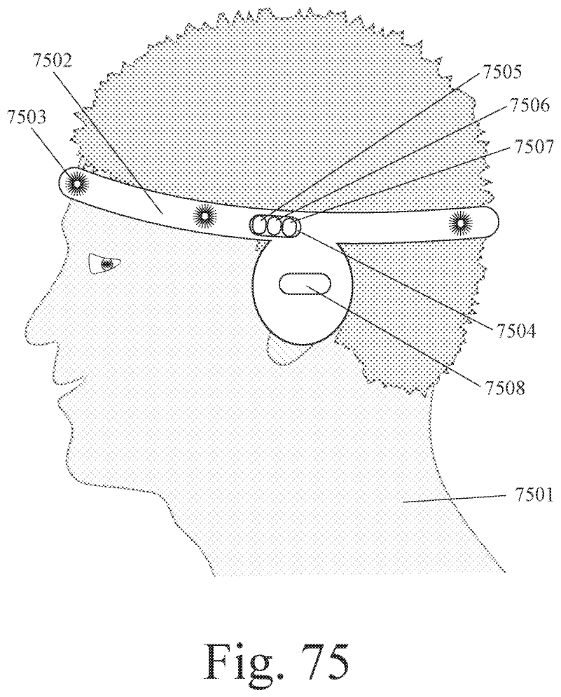

FIG. 75 shows a left-side view of an example of a neurostimulation device comprising a head-worn emitter-positioning member (e.g. frame) 7502 which is configured to position a plurality of electromagnetic energy emitters including 7503 at selected locations on the person's 7501 head. In this example, emitter-positioning member (e.g. frame) 7502 is assumed to be substantially symmetric with respect to the left side (shown) and the right side (not shown) of the person's head. This monitor further comprises control unit 7504, which need not be replicated on the right side. In this example, control unit 7504 further comprises: a data processing component 7505; a power source (or transducer) 7506; and a data transmitting (and receiving) component 7507. In this example, the neurostimulation device further comprises a user interface 7508.

In this example, emitter-positioning member (e.g. frame) 7502 comprises: a ring or band portion which encircles the top of the person's head in a manner like the rim of a cap; and an ear-contacting portion. In an example, an ear-contacting portion can cover a person's ear like the ear-covering portion of traditional headphones. In an example, an ear-contacting portion can be at least partially inserted into a person's ear canal like an earbud or hearing aid. In this example, the ring or band portion and the ear-contacting portion are joined on the left side and right side at locations just over the person's left ear and right ear, respectively.

In an example, this neurostimulation device can comprise an array of electromagnetic energy emitters which are located substantially at the following set of placement sites —CP1, CP2, CP3, CP4, CP5, CP6, CPz, FC1, FC2, FC3, FC4, FC5, FC6, FCz, O1, O2, Oz, P7, P8, PO7, PO8, TP7 and TP8—or a subset of these sites. In an example, the most anterior portion of the frame of a wearable brain activity neurostimulation device can span a person's forehead. In an example, the most anterior portion can hold two or more electromagnetic energy emitters on a person's forehead. In an example, the most anterior portion can hold electromagnetic energy emitters at two or more locations selected from the group consisting of: FP1, FPz, and FP2. In an example, the most anterior portion can hold electromagnetic energy emitters at two or more locations selected from the group consisting of: AF7, AF3, AFz, AF4, and AF8. In an example, an ear-contacting portion can include one or more electromagnetic energy emitters which are held in electromagnetic communication with the person's outer ear, ear canal, or both.

In an example, a ring or band portion (which goes around a person's head) of this neurostimulation device can be circular. In an example, a ring or band portion can have a shape selected from the group consisting of: circle, ellipse, and oval. In an example, a ring or band portion can have vertical undulations or waves around a central circumferential axis, wherein the central circumferential axis is circular, elliptical, or oval. In an example, a ring or band portion can have horizontal and/or radial undulations or waves around a central circumferential axis, wherein the central circumferential axis is circular, elliptical, or oval. In an example, undulations or waves can be sinusoidal. In an example, a ring or band portion can have an ascending-and-descending wave over (and around) a person's ear. In an example, such an ascending-and-descending wave can be sinusoidal.

In an example, a ring or band portion can be generally circular or elliptical, but have an upward (concave) curved portion over (and around) the person's ear. In an example, a “ring-fitting plane” can be defined as the plane which best fits the ring or band portion of this neurostimulation device. In an example, the ring-fitting plane can be horizontal (when a person stands upright). In an example, the ring-fitting plane can intersect a horizontal plane at a forward-facing acute angle. In an example, this angle can be between 5 and 20 degrees. In an example, this angle can be between 15 and 45 degrees.

In an example, an emitter-positioning member (e.g. frame) can be a single continuous structure, even if it has different branches or arms. In an example, an emitter-positioning member (e.g. frame) can have multiple separately-made but connected pieces (such as branches and/or arms). In an example, these pieces can be connected with joints, hinges, or elastic straps. In an example, an emitter-positioning member (e.g. frame) can be flexible and/or elastic. In an example, the size, tension, and/or elasticity of an emitter-positioning member (e.g. frame) can be adjusted. In an example, the size, tension, and/or elasticity of an emitter-positioning member (e.g. frame) can be adjusted by a pneumatic mechanism and/or inflatable chamber. In an example, the size, tension, and/or elasticity of an emitter-positioning member (e.g. frame) can be adjusted by a hydraulic mechanism. In an example, the size, tension, and/or elasticity of an emitter-positioning member (e.g. frame) can be adjusted by a spring mechanism.

In an example, an electromagnetic energy emitter can be made by printing high-conductivity ink onto a low-conductivity textile or fabric. In an example, an electromagnetic energy emitter can be made by printing a conductive elastomeric material onto a low-conductivity textile or fabric. In an example, an electromagnetic energy emitter can be made by melting or adhering elastomeric conductive material onto a low-conductivity textile or fabric. In an example, an electromagnetic energy emitter can be made by embroidering conductive material onto a low-conductivity textile or fabric. In an example, an electromagnetic energy emitter can be made by weaving, knitting, sewing, embroidering, layering, laminating, adhering, melting, fusing, printing, spraying, painting, or pressing electroconductive material into (or onto) a fabric or textile. In an example, electroconductive threads, fibers, yarns, strands, filaments, traces, and/or layers within a fabric or textile can be configured near a person's skin in order to receive electromagnetic energy.

In an example, an electromagnetic energy emitter can be attached to a neurostimulation device using an attachment mechanism selected from the group consisting of: adhesive, band, buckle, button, channel, clasp, clip, electronic connector, flexible channel, hook, hook-and-eye mechanism, magnet, pin, plug, pocket, rivet, sewing, snap, tape, tie, and zipper. In an example, an electromagnetic energy emitter can be attached to a neurostimulation device by printing, laminating, adhering, embroidering, melting, and/or sewing electroconductive material. In an example, an electronically-functional fabric or textile with electromagnetic energy emitters can be created by weaving, knitting, sewing, embroidering, layering, laminating, adhering, melting, fusing, printing, spraying, painting, or pressing together electroconductive threads, fibers, yarns, strands, filaments, traces, and/or layers. In an example, electroconductive threads, yarns, fibers, strands, channels, and/or traces comprising electromagnetic energy emitters can have shapes or configurations which are selected from the group consisting of: circular, elliptical, or other conic section; square, rectangular, hexagon, or other polygon; parallel; perpendicular; crisscrossed; nested; concentric; sinusoidal; undulating; zigzagged; and radial spokes.

In an example, an electronically-functional fabric or textile with electromagnetic energy emitters can be created by printing, spraying, or otherwise depositing electroconductive ink or resin onto an otherwise non-conductive fabric or textile. In an example, an electronically-functional circuit with electromagnetic energy emitters can be created as part of a neurostimulation device by printing a conductive pattern with electroconductive ink or resin. In an example, an electronically-functional fabric or textile with electromagnetic energy emitters can be created by laminating electro-conductive members onto a non-conductive substrate. In an example, an electronically-functional fabric or textile with electromagnetic energy emitters can be created by embroidering a generally non-conductive fabric or textile member with electro-conductive members. In an example, an electronically-functional circuit with electromagnetic energy emitters can be created for a neurostimulation device by embroidering a conductive pattern with electroconductive thread.

In an example, an electromagnetic energy emitter can be made with a low-conductivity material selected from the group consisting of: acetate, acrylic, cotton, denim, elastane, latex, linen, Lycra™, neoprene, nylon, nylon, polyester, wool, silicone, polydimethylsiloxane (PDMS), silk, spandex, and rayon. In an example, an electromagnetic energy emitter can be made with a high-conductivity material selected from the group consisting of: aluminum or aluminum alloy; carbon nanotubes, graphene, or other carbon-based material; copper or copper alloy; gold; nickel; silver; and steel. In an example, an electromagnetic energy emitter can be made with polydimethylsiloxane (PDMS) which has been doped or impregnated with aluminum, carbon (in one or more various configurations and formulations), copper, gold, nickel, silver, or steel. In an example, an electromagnetic energy emitter can be made from polydimethylsiloxane (PDMS) and carbon nanotubes. In an example, an electromagnetic energy emitter can be made from polydimethylsiloxane (PDMS) and silver. Variations discussed elsewhere in this or priority-linked disclosures can also be applied to this example.

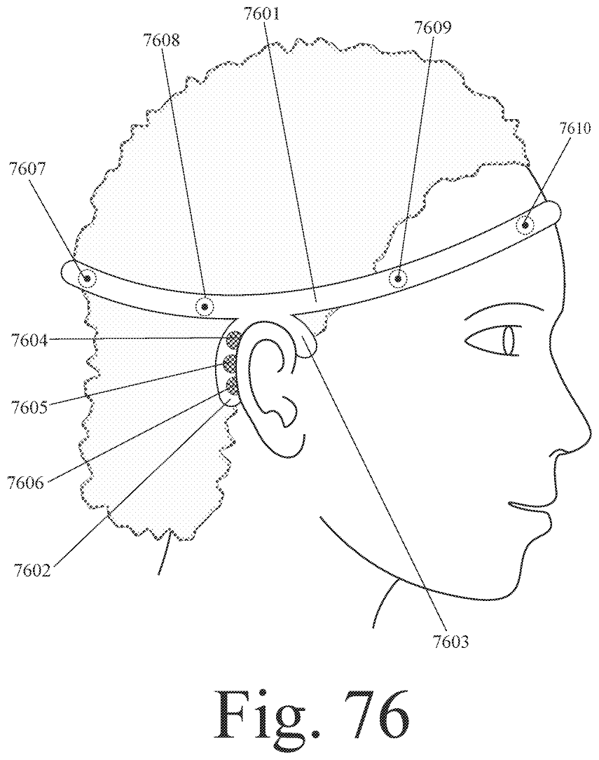

FIG. 76 shows an example of a neurostimulation device comprising: a headband (or halo) 7601; a rearward ear-engaging member 7602; a frontal ear-engaging member 7603; a plurality of electromagnetic energy emitters (including 7607, 7608, 7609, and 7610) which are configured to be held in proximity to the person's head by the headband (or halo); a wireless data transmitter and/or receiver 7604; a data processor 7605; and a power source 7606. In an example, this neurostimulation device can have a symmetric configuration on the other side of the person's head, which is not shown here.

In an example, a headband (or halo) can encircle a person's head, including spanning at least a portion of the person's forehead. In an example, a headband (or halo) can span the center of a person's forehead. In an example, a headband (or halo) can encircle a person's head at a substantially uniform height when the person is standing upright. In an example, a headband (or halo) can encircle a person's head at a substantially uniform distance from the top of the person's head. In an example, a headband (or halo) can have a shape which is selected from the group consisting of: circle, oval, ellipse, and egg-shape.

In an example, a headband (or halo) can be made from a metal and/or a polymer. In an example, a headband (or halo) can be gas permeable (e.g. breathable) and/or liquid permeable. In an example, the perimeter of a headband (or halo) can further comprise a spring or other tensile member which holds the headband (or halo) against the surface of the person's head. In an example, the perimeter of a headband (or halo) can further comprise a spring or other tensile member which causes the headband to exert (modest) pressure against the surface of the person's head to better hold the headband on the person's head and/or better hold the electromagnetic energy emitters in proximity to the surface of the person's head.

In an example, a headband (or halo) can be stretchable, elastic, and/or expandable. In an example, one or more sections of the perimeter of a headband (or halo) can be stretchable, elastic, and/or expandable. In an example, the perimeter of a headband (or halo) can comprise one or more sections with a first degree of stretchability, elasticity, and/or expandability and one or more sections with a second degree of stretchability, elasticity, and/or expandability, wherein the second degree is greater than the first degree. In an example, the rear portion of the perimeter of a headband (spanning the rear of the person's head) can have a first degree of stretchability, elasticity, and/or expandability and the front portion of the perimeter of the headband (spanning the person's forehead) can have a second degree of stretchability, elasticity, and/or expandability, wherein the second degree is greater than the first degree. In an example, the rear portion of the perimeter of a headband (spanning the rear of the person's head) can have a first degree of stretchability, elasticity, and/or expandability and the front portion of the perimeter of the headband (spanning the person's forehead) can have a second degree of stretchability, elasticity, and/or expandability, wherein the second degree is less than the first degree.

In an example, a headband (or halo) can rest on the top portions of a person's ears (or the top portions of the tissue which connects the outer ears to the main body of the head). In an example, a rearward ear-engaging member can be configured to curve around (and frictionally engage) a rear portion of the perimeter of a person's outer ear. In an example, a rearward ear-engaging member can curve around (and frictionally engage) a person's outer ear between the 6 o'clock (210 degree) and 12 o'clock (0 degree) vectors. In an example, a rearward ear-engaging member can curve around (and frictionally engage) a person's outer ear between the 8 o'clock (210 degree) and 12 o'clock (0 degree) vectors. In an example, a rearward ear-engaging member can be configured to span between 20% and 50% of the perimeter of a person's ear. In an example, a rearward ear-engaging member can be configured to be attached to a person's earlobe.

In an example, a frontal ear-engaging member can be configured to curve around (and frictionally engage) a front portion of the perimeter of a person's outer ear. In an example, a frontal ear-engaging member can curve around (and frictionally engage) a person's outer ear between the 12 o'clock (0 degree) and 4 o'clock (120 degree) vectors. In an example, a frontal ear-engaging member can curve around (and frictionally engage) a person's outer ear between the 12 o'clock (0 degree) and 2 o'clock (60 degree) vectors. In an example, a rearward ear-engaging member can be configured to span between 10% and 30% of the perimeter of a person's ear.

FIG. 76 also shows an example of a neurostimulation device comprising: a flexible ring and/or band 7601; an arcuate ear-contacting member (comprising portions 7602 and 7603); a plurality of electromagnetic energy emitters (including 7607, 7608, 7609, and 7610) which are configured to be held in proximity to the person's head by the flexible ring and/or band; a wireless data transmitter and/or receiver 7604; a data processor 7605; and a power source 7606. In an example, a neurostimulation device can comprise: an arcuate flexible ring and/or band which is worn around a person's head, spanning both the person's forehead and the rear of the person's head; an arcuate ear-contacting portion which curves around at least a portion of the person's ear; and a plurality of electromagnetic energy emitters which are held by the neurostimulation device in electromagnetic communication with the person's head.

As shown in FIG. 76, an ear-contacting portion can curve around a portion of a person's ear in an arcuate manner. In an example, the ear-contacting portion can curve around the upper portion of the person's ear in an arcuate manner. In an example, the ear-contacting portion can have a sinusoidal shape. In an example, the ear-contacting portion can have a conic section shape. In an example, the ear-contacting portion can curve around the rear portion of the person's ear in an arcuate manner. In an example, the ear-contacting portion can be attached to the person's earlobe. In an example, the ear-contacting portion can partially protrude into the person's ear canal.

In an example, the flexible band, the ear-contacting portion, and/or the electromagnetic emitters of this neurostimulation device can be made from an elastomeric polymer which has been impregnated, doped, coated, or embedded with conductive material. In an example, the flexible band, ear-contacting portion, and/or electromagnetic emitters of this neurostimulation device can be made from silicone material (such as PDMS) which has been impregnated, doped, coated, or embedded with silver. In an example, the flexible band, ear-contacting portion, and/or electromagnetic emitters of this neurostimulation device can be made from silicone material (such as PDMS) which has been impregnated, doped, coated, or embedded with carbon nanotubes. In an example, the flexible band, ear-contacting portion, and/or electromagnetic emitters of this neurostimulation device can be made from silicone material (PDMS) which has been impregnated, doped, coated, or embedded with graphene.

In an example, a headband (or halo) can be configured to receive the side frame of a pair of eyeglasses so that this headband (or halo) can be worn in combination with eyeglasses. In an example, a headband (or halo) can further comprise an opening which is configured to receive the side-piece of an eyeglass frame so that this headband (or halo) can be worn in combination with eyeglasses. In an example, a headband (or halo) can further comprise a clip or other attachment mechanism to which the side-piece of an eyeglass frame can be attached so that this headband (or halo) can be worn in combination with eyeglasses. In an example, headband (or halo) can further comprise an indentation, groove, or track into (or against) which the side-piece of the side-piece of an eyeglass frame can be placed so that this headband (or halo) can be worn in combination with eyeglasses. Variations discussed elsewhere in this or priority-linked disclosures can also be applied to this example.

I claim:

1. A neurostimulation device comprising:
a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and
a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy emitters are made from an elastic polymer.

2. A neurostimulation device comprising:
a wearable ring and/or band which is configured to encircle a person's head at an anterior acute angle in the range of 0 to 45 degrees with respect to a horizontal plane when the person's head is upright; and
a plurality of electromagnetic energy emitters on the band; wherein one or more of the electromagnetic energy emitters are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy emitters are made from a polymer which has been impregnated, doped, coated, or embedded with conductive material; and
wherein one or more of the electromagnetic energy emitters have prongs and/or teeth which protrude into and/or under the person's hair.

3. A neurostimulation device comprising:
a wearable ring and/or band which is configured to encircle a person's head at an anterior acute angle in the range of 0 to 45 degrees with respect to a horizontal plane when the person's head is upright; and
a plurality of electromagnetic energy emitters on the band; wherein one or more of the electromagnetic energy emitters are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy emitters are made from a silicone material which has been impregnated, doped, coated, or embedded with silver or carbon; and wherein one or more of the electromagnetic energy emitters have prongs and/or teeth which protrude into and/or under the person's hair.
